(19) United States
(12) Patent Application Publication
(10) Pub. No.: US 2024/0344145 A1
(43) Pub. Date: Oct. 17, 2024

(54) PHARMACOGENOMICS SCORE TO MAKE DECISIONS ON THERAPY AUGMENTATION IN AML

(71) Applicants: University of Florida Research Foundation, Incorporated, Gainesville, FL (US); St. Jude Children's Research Hospital, Inc., Memphis, TN (US); University of Tennessee Research Foundation, Knoxville, TN (US)

(72) Inventors: Jatinder Kaur Lamba, Gainesville, FL (US); Abdelrahman H. Elsayed, Gainesville, FL (US); Xueyuan Cao, Memphis, TN (US); Stanley Pounds, Memphis, TN (US)

(73) Assignees: University of Florida Research Foundation, Incorporated, Gainesville, FL (US); St. Jude Children's Research Hospital, Inc., Memphis, TN (US); University of Tennessee Research Foundation, Knoxville, TN (US)

(21) Appl. No.: 18/683,969

(22) PCT Filed: Aug. 15, 2022
(86) PCT No.: PCT/US2022/040326

§ 371 Date: Feb. 15, 2024

Related U.S. Application Data
(60) Provisional application No. 63/233,673, filed on Aug. 16, 2021.

(51) Int. Cl. C12Q 1/6886; C12Q 1/6874

(52) U.S. Cl. CPC C12Q 1/6886; C12Q 1/6874; C12Q 2600/106; C12Q 2600/156

(57) ABSTRACT

The disclosure relates to methods for characterizing and/or treating a subject having cancer, said methods comprising performing an assay to identify the nucleotides present at each of a set of single-nucleotide polymorphism (SNP) locations within the cytarabine (ara-C) pathway, assigning a genotype score for the identified nucleotides of each SNP, and characterizing the subject having cancer based on the summation of the assigned genotype scores. In some embodiments, treatment is administered based upon the characterization of the subject, according to the methods described herein.

### RELATED APPLICATIONS

This application claims the benefit under 35 U.S.C. § 119 (e) of the filing date of U.S. provisional Application Ser. No. 63/233,673, filed Aug. 16, 2021, entitled “PHARMACOGENOMICS SCORE TO MAKE DECISIONS ON THERAPY AUGMENTATION IN AML”, the entire contents of which are incorporated by reference herein.

### STATEMENT OF GOVERNMENT SUPPORT

This invention was made with government support under CA132946 awarded by the National Institutes of Health. The government has certain rights in the invention.

### FIELD OF THE INVENTION

The disclosure relates, at least in part, to single nucleotide polymorphisms (SNPs) that can be used to predict whether or not a subject with cancer may benefit from a particular treatment.

### BACKGROUND OF INVENTION

Cytarabine, also known as ara-C, is a backbone of chemotherapy for certain types of cancers, including, for example, certain types of leukemia. However, in some instances, subjects having a leukemia exhibit poor outcomes when administered a standard chemotherapy regimen which includes, among other agents, cytarabine. Other subjects having a leukemia respond well to such standard regimens. Accordingly, standard chemotherapeutic regimens are not appropriate for all subjects. A solution is needed which characterizes a subject having cancer such that a personalized treatment plan, including the administration of cytarabine at a dosage tailored to the subject's genotype, can be developed to treat the cancer.

### SUMMARY OF INVENTION

Aspects of the disclosure relate to methods for characterizing subjects having cancer, and for treating said subjects based on the characterizing. In some embodiments, the cancer is a leukemia.

In some embodiments, a method for characterizing a subject having cancer comprises the steps of (i) assigning a genotype score based upon the nucleotides present at each of a set of single-nucleotide polymorphism (SNP) locations comprising rs10916819, rs17103168, rs5841, rs2396243, rs1044457, rs1138729, rs4643786, rs11030918, rs12067645, and rs17343066 in a biological sample obtained from the subject, and (ii) characterizing the subject having cancer based on the summation of the assigned genotype scores of (i). In some embodiments, the genotype score is assigned according to a method comprising:
(a) assigning a genotype score for the SNP location rs10916819 of zero (0) when the characterized nucleotides are AA, or negative one (−1) when the characterized nucleotides are AG or GG;
(b) assigning a genotype score for the SNP location rs17103168 of zero (0) when the characterized nucleotides are AA, or one (1) when the characterized nucleotides are AG or GG;
(c) assigning a genotype score for the SNP location rs5841 of zero (0) when the characterized nucleotides are CC, or one (1) when the characterized nucleotides are CT or TT;
(d) assigning a genotype score for the SNP location rs2396243 of zero (0) when the characterized nucleotides are GG, negative one (−1) when the characterized nucleotides are AG, or negative two (−2) when the characterized nucleotides are AA;
(e) assigning a genotype score for the SNP location rs1044457 of zero (0) when the characterized nucleotides are CC, or one (1) when the characterized nucleotides are CT or TT;
(f) assigning a genotype score for the SNP location rs1138729 of zero (0) when the characterized nucleotides are AA, or negative one (−1) when the characterized nucleotides are AG or GG;
(g) assigning a genotype score for the SNP location rs4643786 of zero (0) when the characterized nucleotides are TT, negative one (−1) when the characterized nucleotides are CT, or negative two (−2) when the characterized nucleotides are CC;
(h) assigning a genotype score for the SNP location rs11030918 of zero (0) when the characterized nucleotides are TT or CT, or one (1) when the characterized nucleotides are CC;
(i) assigning a genotype score for the SNP location rs12067645 of zero (0) when the characterized nucleotides are GG, one (1) when the characterized nucleotides are AG, or two (2) when the characterized nucleotides are AA; and
(j) assigning a genotype score for the SNP location rs17343066 of zero (0) when the characterized nucleotides are GG or AG, or one (1) when the characterized nucleotides are AA.

In some embodiments, the methods described herein further comprise performing an assay to identify the nucleotides present at each of the set of SNP locations, wherein the assay is performed prior to (i), as described above. In some embodiments, the assay is performed by DNA sequencing analysis, using a hybridization assay, using a Sequenom MassARRAY platform, or using a TaqMan genotyping assay.

In some embodiments, the genotype score is assigned based on information previously obtained from the sample. In some embodiments, the summation of the assigned genotype scores is calculated by adding the genotype scores assigned according to the method of (a)-(j), as described above.

In some embodiments, the cancer is acute lymphoblastic leukemia (ALL), acute promyelocytic leukemia (APL), Chronic Myelogenous Leukemia (CML), or acute myeloid leukemia (AML). In some embodiments, the AML is pediatric AML. In some embodiments, the subject is less than 19 years of age. In some embodiments, the subject is a pediatric subject. In some embodiments, the subject is an adult subject.

In some embodiments, the subject was administered one or more chemotherapeutic agents prior to the characterizing. In some embodiments, the methods described herein further comprise administering a chemotherapeutic agent to the subject after the characterizing. In some embodiments, the chemotherapeutic agent comprises cytarabine (ara-C), daunorubicin hydrochloride, and/or etoposide phosphate (ADE). In some embodiments, the subject is administered cytarabine at a high dose when the summation of the assigned genotype scores is less than or equal to zero (0). In some embodiments, the subject is administered cytarabine at a low dose when the summation of the assigned genotype scores is greater than zero (0). In some embodiments, the subject is further administered an agent that selectively binds to CD33 when the summation of the assigned genotype scores is less than or equal to zero (0).

In some embodiments, the methods described herein further comprise (iv) performing an assay to detect the genotype of the subject for the SNP rs12459419, wherein the genotype may be CC. TC, or TT. In some embodiments, the assay is performed by DNA sequencing analysis, using a hybridization assay, using a Sequenom MassARRAY platform, or using a TaqMan genotyping assay. In some embodiments, the methods described herein further comprise (v) administering a therapeutically effective amount of an agent that selectively binds to CD33 when the subject exhibits a CC genotype for the CD33 single-nucleotide polymorphism rs12459419.

In some embodiments, the agent that selectively binds to CD33 is gemtuzumab ozogamicin (GO), hP67.7, SGN-33A, or an antibody that selectively binds CD33 or an antigen binding fragment thereof. In some embodiments, the agent that selectively binds to CD33 is GO. In some embodiments, the antibody that selectively binds CD33 is a humanized antibody. In some embodiments, the agent that selectively binds to CD33 comprises an antibody that selectively binds CD33, or an antigen binding fragment thereof, conjugated to a toxin. In some embodiments, the agent that selectively binds to CD33 selectively binds to amino acids encoded by exon 2 of CD33.

In some embodiments, the subject is treated with a chemotherapeutic agent within thirty days of the administration of the agent that selectively binds to CD33. In some embodiments, the chemotherapeutic agent comprises cytarabine (Ara-C), daunorubicin hydrochloride, and/or etoposide phosphate.

In some embodiments, the subject has one or more of: the presence of blast cells that express CD33 within the hematopoietic system; leukostasis; anemia; leukopenia; neutropenia; thrombocytopenia; chloroma; granulocytic sarcoma; and myeloid sarcoma.

Aspects of the disclosure relate to a method of treating a subject having cancer, the method comprising (i) administering to the subject a high dose of cytarabine when the summation of the assigned genotype scores is less than or equal to zero (0); or (ii) administering to the subject a low dose of cytarabine when the summation of the assigned genotype scores is greater than zero (0). In some embodiments, the genotype score is assigned by characterizing the subject having cancer according to any one of the methods as described herein.

In some embodiments, the subject was administered a chemotherapeutic agent prior to the treating. In some embodiments, the methods described herein further comprise administering a chemotherapeutic agent to the subject concurrently with or after the treating. In some embodiments, the chemotherapeutic agent comprises cytarabine (Ara-C), daunorubicin hydrochloride, etoposide phosphate, and/or an agent that selectively binds to CD33.

In some embodiments, the agent that selectively binds to CD33 is administered when the summation of the assigned genotype scores is less than or equal to zero (0) and/or when the subject exhibits a CC genotype for the SNP rs12459419. In some embodiments, the agent that selectively binds to CD33 is gemtuzumab ozogamicin (GO), hP67.7, SGN-33A, or an antibody that selectively binds CD33 or an antigen binding fragment thereof. In some embodiments, the agent that selectively binds to CD33 is GO. In some embodiments, the antibody that selectively binds CD33 is a humanized antibody. In some embodiments, the agent that selectively binds to CD33 comprises an antibody that selectively binds CD33, or an antigen binding fragment thereof, conjugated to a toxin. In some embodiments, the agent that selectively binds to CD33 selectively binds to amino acids encoded by exon 2 of CD33.

### BRIEF DESCRIPTION OF THE DRAWINGS

FIG. 1 shows a schematic depicting overall study designs described in Examples 1 and 2.

FIGS. 2A-2G show patient outcomes by Composite ACS10 Score groups. FIG. 2A shows event free survival (EFS) in AML02 cohort. FIG. 2B shows overall survival (OS) in AML02. FIG. 2C shows minimal residual disease after induction I course of treatment (MRD1) in AML02 cohort. FIG. 2D shows remission status after induction I course of treatment in AML02 cohort. FIG. 2E shows EFS in COG AAML0531 ADE arm validation cohort. FIG. 2F shows OS in COG AAML0531 ADE arm. FIG. 2G shows MRD1 in COG AAML0531 ADE arm.

FIGS. 3A-3D show forest plots of multivariable cox proportional hazard models that include ACS10 score groups, risk-group assignment, race, white blood cell count (WBC) at diagnosis, and age for association with patient outcomes. FIG. 3A shows EFS in AML02 cohort. FIG. 3B shows OS in AML02 cohort. FIG. 3C shows EFS and FIG. 3D shows OS, both in COG-AAML0531 cohort (standard chemotherapy ADE arm).

FIGS. 4A-4I show the impact of ACS10 Score groups on outcome within LDAC and HDAC treatment arms of AML02 cohort. FIG. 4A shows EFS in low dose (LDAC) arm. FIG. 4B shows OS in LDAC arm. FIG. 4C shows EFS in high dose (HDAC) arm. FIG. 4D shows OS in HDAC arm. FIGS. 4E-4H show forest plots of multivariable cox proportional hazard models that includes ACS10 score groups, risk-group assignment, race, white blood cell count (WBC) at diagnosis, and age for association with patient outcomes. FIG. 4E shows EFS and FIG. 4F shows OS, both in AML02-LDAC arm. FIG. 4G shows EFS and FIG. 4H shows OS, both in AML02-HDAC arm. FIG. 4I shows impact of interaction between numerical ACS10 scores and treatment arms (LDAC vs. HDAC) on 3-year survival in AML02 cohort.

FIGS. 5A-5C show a comparison of patient outcomes by ACS10 Score groups and treatment arm in AAML0531 cohort (total 931 patients; ADE arm, N=465 and ADE+GO arm, N=466). FIG. 5A shows EFS. FIG. 5B shows OS. Solid lines represent patients from ADE arm and dashed lines represent patients in ADE+GO arm. FIG. 5C shows impact of interaction between numerical ACS10 scores and treatment arms (ADE vs. ADE+GO) on 3-year survival in COG-AAML0531 cohort.

FIGS. 6A-6F show patient outcomes by genotypes of the six SNPs found significantly associated with MRD1 and EFS in multivariable SNP combination models. MRD1 by CDA rs10916819 (FIG. 6A); CMPK1 rs17103168 (FIG. 6B); NME4 rs5841 (FIG. 6C); EFS by RRM2 rs1138729 (FIG. 6D); CMPK1 rs1044457 (FIG. 6E) and SLC29A1 SNP rs2396243 (FIG. 6F).

FIGS. 7A and 7B show histograms showing frequency distribution of ACS10 Scores. FIG. 7A shows AML02 discovery cohort (N=166 patients), and FIG. 7B shows AAML0531-Standard ADE arm validation cohort (N=465 patients).

FIGS. 8A and 8B show forest plots of multivariable Cox proportional hazard models that include Composite ACS10 Score groups, risk group assignment, white blood cell count (WBC), and age at diagnosis for association. FIG. 8A shows MRD1 in AML02 and FIG. 8B shows MRD1 in COG-AAML0531 (ADE arm).

FIGS. 9A-9D show interaction between ACS10 Score groups and MRD1 status in AML02. FIG. 9A shows EFS by MRD1 status (positive or negative) in patients with Low or High ACS10 Score groups, and FIG. 9B shows OS by MRD1 status in patients with Low or High ACS10 Score groups in AML02 cohort. FIGS. 9C and 9D represent EFS and OS by MRD status in AAML0531-COG cohort (ADE arm).

FIG. 10 shows the impact of ACS10 Score groups on remission status after induction I within LDAC and HDAC treatment arms of AML02 cohort.

FIGS. 11A and 11B show the impact of interaction between numerical ACS10 scores and treatment arms (LDAC vs. HDAC) on 4- and 5-year survival in AML02 cohort. FIG. 11A shows ACS10 scores and treatment arm interaction at 4- and 5-year OS (top and bottom, respectively) in AML02. FIG. 11B shows ACS10 scores and treatment arm interaction at 4- and 5-year EFS (top and bottom, respectively) in AML02.

FIGS. 12A-12C show ACS10 Score by ADE vs. ADE+GO treatment arms in COG-AAML0531. FIG. 12A shows EFS and FIG. 12B shows OS by ACS10 score groups in patients treated with GO containing regimen in COG-AAML0531 (ADE+GO arm; N=466). FIG. 12C shows the impact of ACS10 Score groups on MRD1 status within ADE and ADE+GO arms of COG-AAML0531 cohort.

FIGS. 13A-13C show forest plots of multivariable Cox proportional hazard model within low ACS10 score group that treatment arm (ADE/ADE+GO) with inclusion of risk group assignment, white blood cell count (WBC), and age at diagnosis for association. FIG. 13A shows EFS. FIG. 13B shows OS, and FIG. 13C shows MRD1, all within low ACS10 group.

FIGS. 14A and 14B shows impact of interaction between numerical ACS10 scores and treatment arms ADE vs. ADE+GO on 4- and 5-year survival in AML02 cohort. FIG. 14A shows 4- (top) and 5-year (bottom) survival for OS in AML02. FIG. 14B shows 4- (top) and 5-year (bottom) survival for EFS in AML02.

FIGS. 15A-15J show patient outcomes by Composite ACS10 Score groups in the highly heterogeneous standard risk group of patients. FIG. 15A shows event free survival (EFS) in AML02 cohort. FIG. 15B shows overall survival (OS) in AML02. FIG. 15C shows minimal residual disease after induction I course of treatment (MRD1) in AML02 cohort. FIG. 15D shows remission status after induction I course of treatment in AML02 cohort. FIG. 15E shows OS in COG-AAML0531-ADE arm validation cohort. FIG. 15F shows MRD1 in COG-AAML0531-ADE arm. FIGS. 15F-15I show forest plots of multivariable Cox proportional hazard models that includes Composite ACS10 Score groups, race, white blood cell count (WBC) at diagnosis, and age for association with patient outcomes within standard risk group patients. FIG. 15G shows EFS and FIG. 15H shows OS, both in AML02 cohort. FIG. 15I shows EFS and FIG. 15J shows OS, both in COG-AAML0531 cohort (ADE arm).

FIG. 16 shows ara-CTP level at day 1 by ACS10 SNP score for the AML97 cohort. Seventy patients exhibit ara-CTP level data at day 1 of treatment. Some patients are missing genotyping data of some of the 10 SNPs; however ACS10 score was able to be calculated for 63 patients for whom the score group did not change as a result of the missing SNPs genotype data.

FIGS. 17A-17C show EFS. OS, and MRD1 within low ACS10 score group by treatment arm. FIG. 17A shows EFS. FIG. 17B shows OS. FIG. 17C shows MRD1.

FIGS. 18A-18D show data relating to the high ACS10 score group by treatment arm. FIG. 18A shows EFS and FIG. 18B shows OS in high ACS10 group by treatment arm. FIGS. 18C and 18D show forest plots of EFS (FIG. 18C) and OS (FIG. 18D) in COG-ADE+GO arm.

FIGS. 19A-19F show the impact of CD33 splicing SNP and ACS10 SNP score on clinical response in patients treated with ADE+GO or ADE alone in a AAML0531 clinical trial. FIG. 19A shows 3-year OS with 10SNP score by treatment arm, genotype, and risk. COG. FIG. 19B shows 4-year OS with 10SNP score by treatment arm, genotype, and risk, COG. FIG. 19C shows 5-year OS with 10SNP score by treatment arm, genotype, and risk. COG. FIG. 19D shows 3-year EFS with 10SNP score by treatment arm, genotype, and risk. COG. FIG. 19E shows 4-year EFS with 10SNP score by treatment arm, genotype, and risk, COG. FIG. 19F shows 5-year EFS with 10SNP score by treatment arm, genotype, and risk, COG.

FIGS. 20A-20C show representative data for ACS10 scoring and analysis of clofarabine (Clo) treated cohorts. FIG. 20A shows a schematic depicting the study design described in Example 3. FIG. 20B shows representative data for event free survival (EFS) and overall survival (OS) by ACS10 numeric value for Clo+Ara-C and LDAC (standard ADE with low dose Ara-C induction). FIG. 20C shows representative data indicating therapy augmentation with Clo+Ara-C in the low ACS10 group (e.g., ACS10≤0) improves therapeutic outcome, whereas therapy augmentation with Clo+Ara-C in the high ACS10 group (e.g., ACS10>0) is detrimental to therapeutic outcome. For high ACS10 score patients, LDAC is a better therapeutic option.

### DETAILED DESCRIPTION OF THE INVENTION

Acute myeloid leukemia (AML) is a heterogenous disease with overall suboptimal outcome. Although chemotherapy regimens that include cytarabine (ara-C) induce remission in a majority of pediatric AML patients, approximately 30% relapse and subsequently have very dismal outcome. Ara-Cis a prodrug that requires activation to ara-C triphosphate (ara-CTP) which induces leukemic cell death. Thus, inter-patient variation in each patient's ability to activate ara-C is a significant contributor to clinical outcomes.

The inventors have recognized and appreciated that certain standard-of-care therapeutic regimens (e.g., standard ADE) used for treating AML patients are not always effective, and that the lack of effectiveness of these regimens arises from the presence of certain single nucleotide polymorphisms (SNPs) of genes associated with the Ara-C pathways of those subjects. Subjects having AML may therefore be evaluated for the presence of these SNPs and administered a therapeutic regimen based upon the outcome of the evaluation (e.g., a score produced using the detection of the presence or absence of such SNPs). The inventors have further recognized and appreciated that patients having scores below a certain threshold (e.g., an ACS10 score of ≤0, “low ACS10” subjects) have improved therapeutic response to high-dose Ara-C induction of ADE therapy or administration of clofarabine, compared to subjects having scores above a certain threshold (e.g., an ACS10 score of >0, “high ACS10” subjects). The inventors have further recognized and appreciated that administration of high-dose Ara-C or clofarabine to high ACS10 subjects may have a detrimental therapeutic effect, and that such subjects should be administered standard low-dose Ara-C ADE therapy. These recognitions by the inventors serve to improve the technology of cancer therapeutic selection for AML patients by selecting subsets of patients who have an increased likelihood of responding to therapeutic augmentation (e.g., via high-dose Ara-C, GO, and/or clofarabine), or by excluding patients that are not likely to response to therapeutic augmentation.

Aspects of the disclosure relate to characterizing a subject having cancer based upon the specific nucleotides present at each of a set of identified single nucleotide polymorphisms (SNPs) of one or more genes within the ara-C pathway. The disclosure is based, in part, on the surprising discovery that certain subjects exhibiting specific nucleotides at certain SNPs respond differently to the administration of chemotherapeutic regimens comprising cytarabine (e.g., ara-C).

In some embodiments, a subject (or biological sample of a subject) is characterized by assigning a genotype score (referred to herein as an “ACS10 score”) to the subject (or biological sample) based upon the nucleotides present at each of the identified SNPs in the ara-C pathway, said ACS10 score being calculated according to a set of criteria as described herein. In some embodiments, a subject is treated for the cancer, or an existing treatment for the cancer is modified, based on the assigned ACS10 score. In some embodiments, the assigned ACS10 score is predictive of cancer prognosis and treatment outcomes.

### Assays

Aspects of the disclosure relate to methods for performing an assay to genotype (e.g., identify the nucleotides present at) certain chromosomal locations (e.g., single nucleotide polymorphisms (SNPs)) within genes of interest (e.g., certain genes within the ara-C pathway) in a biological sample. In some embodiments, the assays described herein to genotype SNPs of interest utilize complementary probes which selectively hybridize to genes of interest in the ara-C pathway. In some embodiments, gene-specific probes selectively hybridize to a gene selected from CDA, CMPK1, NME4, SLC29A1, RRM2, DCK, RRM1, CTPS1, and SLC28A3.

As will be understood, a subject's “genotype” is the collection of genetic material unique to the subject. “Genotyping” is the process of characterizing the genotype of a subject by examining a DNA sequence of the subject and, in some embodiments, comparing it to either a DNA sequence of a second subject or to a reference sequence. Genotyping is performed through the use of biological assays. Suitable assays for genotyping are known in the art, and may generally include restriction fragment length polymorphism identification (RFLPI) of genomic DNA, random amplified polymorphic detection (RAPD) of genomic DNA, amplified fragment length polymorphism detection (AFLPD), polymerase chain reaction (PCR), DNA sequencing, allele specific oligonucleotide (ASO) probes, and hybridization to DNA microarrays or beads, among other methods. Certain assays suitable for use in the present methods are also described herein; however, any suitable assay may be used.

In some aspects, methods described by the disclosure include extraction and/or isolation of nucleic acids (e.g., DNA, RNA, miRNA, etc.) from a biological sample. Methods of extracting nucleic acids from a sample are known, for example as described in Ali et al. (2017) Biomed Res Int.: 9306564. In some embodiments, DNA is extracted from a biological sample. In some embodiments, DNA is extracted from a biological sample using a commercially available DNA extraction kit, such as Masterpure™ Complete DNA and RNA Purification Kit. In some embodiments, methods described herein comprise a step of amplifying the DNAs to produce amplification products, also referred to as “amplicons”.

In some embodiments, a SNP is genotyped using DNA sequencing analysis. In some embodiments, an SNP is genotyped using nucleic acid sequencing (e.g., DNA sequencing, RNA sequencing, etc.). Examples of sequencing methods used for gene expression profiling include but are not limited to single-molecule real-time sequencing (SMRT), ion semiconductor (Ion Torrent) sequencing, pyrosequencing, sequencing by synthesis (e.g., Illumina sequencing), sequencing by ligation (SOLID), and chain termination sequencing (Sanger sequencing), nanopore sequencing (e.g., Oxford Nanopore sequencing), and massively parallel sequencing (MPSS). Sequencing methods generally utilize gene specific probes (e.g., oligonucleotides, primers, adaptors, etc.) for nucleic acid amplification. In some embodiments, the DNA sequencing analysis comprises high-throughput DNA sequencing (HTS). Methods of using HTS for SNP genotyping are known in the art, for example as described in Altmann, et al., (2012) A beginners guide to SNP calling from high-throughput DNA-sequencing data, Hum Genet 131:1541-54, which is incorporated by reference herein with respect to the disclosure relating to using HTS for SNP genotyping.

In some embodiments, a SNP is genotyped using a hybridization assay. As used herein, the term “hybridization” is accorded its general meaning in the art and refers to the pairing of substantially complementary nucleotide sequences (for example, pairing of oligonucleotides and strands of nucleic acid) to form a duplex or heteroduplex through formation of hydrogen bonds between complementary base pairs in accordance with Watson-Crick base pairing. Hybridization is a specific, i.e., non-random, interaction between two complementary polynucleotides. As will be understood by the skilled person, a hybridization assay comprises any form of quantifiable hybridization (e.g., the quantitative annealing of two complementary strands of nucleic acids, known as nucleic acid hybridization). In some embodiments, complementary DNA probes are hybridized to the SNP site. Examples of assays which utilize hybridization to genotype the SNP are dynamic allele-specific hybridization (DASH), molecular beacons, and SNP microarrays. The DASH method for SNP genotyping is known in the art, and is described, for example, in Howell, et al., (1999) Dynamic allele-specific hybridization. A new method for scoring single nucleotide polymorphisms, Nat Biotechnol 17 (1): 87-8, which is incorporated by reference herein with respect to the disclosure relating to DASH assays for SNP genotyping. Briefly, DASH utilizes the differences in the melting temperature in DNA that results from the instability of mismatched base pairs. In the first step, a genomic segment is amplified and attached to a bead through a PCR reaction with a biotinylated primer. In the second step, the amplified product is attached to a streptavidin column and washed with NaOH to remove the unbiotinylated strand. An allele-specific oligonucleotide is then added in the presence of a molecule that fluoresces when bound to double-stranded DNA. The intensity is then measured as temperature is increased until the melting temperature (Tm) can be determined. A SNP will result in a lower than expected Tm. Molecular beacons for SNP genotyping are known in the art, and make use of a specifically engineered single-stranded oligonucleotide probe (a “molecular beacon”). The unique design of these molecular beacons allows for a simple diagnostic assay to identify SNPs at a given location. If a molecular beacon is designed to match a wild-type allele and another to match a mutant of the allele, the two can be used to identify the genotype of an individual. SNP microarrays for SNP genotyping are known in the art, and comprise hundreds of thousands of probes arrayed on a small chip. Hybridization of the probes to the target sequence of interest, or to a control sequence, allows for many SNPs to be analyzed simultaneously. SNP microarray chips are commercially available, for example the Affymetrix™ Genome-Wide Human SNP Array 6.0 (ThermoFisher Scientific, Catalog Number 901153), which features 1.8 million genetic markers, including more than 906,600 single nucleotide polymorphisms (SNPs) and more than 946,000 probes for the detection of copy number variation.

In some embodiments, a SNP is genotyped using a Sequenom MassARRAY platform. Sequenom MassARRAY platforms for SNP genotyping are known in the art, for example as described in Gabriel, et al., (2009) SNP genotyping using the Sequenom MassARRAY iPLEX platform, Curr Protoc Hum Genet 2:2.12, and in Gabriel and Ziaugra, (2004) SNP genotyping using Sequenom MassARRAY 7K platform, Curr Protoc Hum Genet 2:2.12, which are incorporated by reference herein with respect to the disclosure relating to Sequenom MassARRAY platforms for SNP genotyping. Briefly, Sequenom MassARRAY assay consists of an initial locus-specific PCR reaction, followed by single base extension using mass-modified dideoxynucleotide terminators of an oligonucleotide primer which anneals immediately upstream of the polymorphic site of interest. Using MALDI-TOF mass spectrometry, the distinct mass of the extended primer identifies the SNP allele.

In some embodiments, a SNP is genotyped using a TaqMan® genotyping assay. TaqMan® genotyping assays for SNP genotyping are known in the art, for example as described in Shen, et al., (2009) The TaqMan Method for SNP Genotyping, In: Komar A. (eds) Single Nucleotide Polymorphisms. Methods in Molecular Biology™ (Methods and Protocols), vol 578. Humana Press, Totowa, NJ, and in de la Vega, et al., (2005) Assessment of two flexible and compatible SNP genotyping platforms: TaqMan® SNP Genotyping Assays and the SNPlex™ Genotyping System, Mut Res/Fund and Mol Mech of Mutagenesis 573:1-2, pp. 111-35, which are incorporated by reference herein with respect to the disclosure relating to TaqMan® genotyping assays for SNP genotyping. Briefly, the TaqMan® SNP Genotyping Assay is a single-tube PCR assay that exploits the 5′ exonuclease activity of AmpliTaq Gold® DNA Polymerase. The assay includes two locus-specific PCR primers that flank the SNP of interest, and two allele-specific oligonucleotide TaqMan® probes. These probes have a fluorescent reporter dye at the 5′ end, and a non-fluorescent quencher (NFQ) with a minor groove binder (MGB) at the 3′ end. The use of two probes, one specific to each allele of the SNP and labeled with two fluorophores, allows detection of both alleles in a single tube. The TaqMan SNP Genotyping Assay is read at the PCR endpoint rather than in real time. DNA samples are genotyped simultaneously on 96- or 384-well plates. Genotype calls for individual samples are made by plotting the normalized intensity of the reporter dyes in each sample well on a cartesian plot. A clustering algorithm in the data analysis software assigns individual sample data to a particular genotype cluster.

In some embodiments, a SNP is genotyped using a microarray assay. Microarray assays are known, for example as described in Bumgartner (2013) Curr Protoc Mol Biol. 2013 January; 0 22: Unit-22.1. Examples of commercially available microarray assays include Affymetrix GeneChip, Illumina BeadArray, Agilent microarrays, etc. Generally, a microarray assay comprises the steps of detecting the presence or absence of an interaction between a sample (e.g., a nucleic acid such as DNA present in a sample) and a material at each location on a substrate. Various methods of detecting an interaction are recognized in the art. For example, interaction between the sample and the material can be detected by measuring binding activity between the sample and the material. As used herein, the term “binding activity” refers to the chemical linkage formed between two molecules. For example, a protein ligand may become covalently bound to its cognate receptor via the chemical interaction between the amino acid residues of the ligand and the receptor. In the context of nucleic acid interactions, binding activity includes the hybridization of complementary nucleic acids.

Other assays known in the art are also envisaged for SNP genotyping, for example comprising enzyme-based methods (restriction fragment length polymorphism (RFLP); PCR-based methods (e.g., tetra-primer amplification refractory mutation system PCR (ARMS-PCR); quantitative PCR (qPCR)); flap endonuclease (FEN) (e.g., using the Invader assay); primer extension (e.g., using complementary probes which may be detected by, e.g., MALDI-TOF mass spectrometry and ELISA-like methods; arrayed primer extension (APEX; APEX-2); Illumina Incorporated's Infinium assay); oligonucleotide ligation assay (e.g., using complementary probes which may be detected by, e.g., gel electrophoresis, MALDI-TOF mass spectrometry or by capillary electrophoresis for large-scale applications); single strand conformation polymorphism; temperature gradient gel electrophoresis (TGGE); temperature gradient capillary electrophoresis (TGCE); denaturing high performance liquid chromatography (DHPLC); high resolution melting analysis; through the use of DNA mismatch-binding proteins; SNPlex™ assay (Applied Biosystems); or surveyor nuclease assay.

In some embodiments, an assay is performed to genotype a SNP(s) of interest prior to practicing the methods of the present disclosure. In such embodiments, the methods described herein comprise characterizing and/or treating a subject having cancer based upon the SNP genotype data previously obtained, and do not comprise performing an assay to genotype a SNP(s) of interest, as described herein.

### Biological Samples and Subjects

Aspects of the disclosure relate to assays performed on biological samples obtained from subjects having cancer. Generally, a biological sample can be blood, serum (e.g., plasma from which the clotting proteins have been removed), or cerebrospinal fluid (CSF). However, the skilled artisan will recognize other suitable biological samples, such as certain tissue (e.g., bone marrow, brain tissue, spinal tissue, etc.) and cells (e.g., leukocytes, stem cells, brain cells, neuronal cells, skin cells, etc.). In some embodiments, a biological sample is a blood sample or a tissue sample. In some embodiments, a blood sample is a sample of whole blood, a plasma sample, or a serum sample. In some embodiments, a tissue sample is a bone marrow tissue sample. In some embodiments, a blood sample is treated to remove white blood cells (e.g., leukocytes), such as the buffy coat of the sample. In some embodiments, a biological sample is obtained from a leukemia patient (e.g., a human leukemia patient). In some embodiments, a tissue sample comprises bone marrow cells and/or leukemic blast cells. In some embodiments, a tissue sample comprises bone marrow aspirate.

As used herein, the term “subject” (or “patient”) refers to an animal having or suspected of having a disease, or an animal that is being tested for a disease. In some embodiments, the subject is selected from the group consisting of human, non-human primate, rodent (e.g., mouse or rat), canine, feline, or equine. In some embodiments, the subject is a human. In some embodiments a human subject is an adult (e.g., an individual over the age of 18). In some embodiments a subject is a child (e.g., a pediatric subject) that is less than 18 years of age. In some embodiments, a subject was administered one or more chemotherapeutic agents prior to being characterized and/or treated according to the methods as described herein. In some embodiments, the chemotherapeutic agents comprise cytarabine (ara-C), daunorubicin, etoposide, or the combination of these drugs-which is referred to as “ADE”. Other examples of chemotherapeutic agents include, but are not limited to, Arsenic Trioxide, Cerubidine (Daunorubicin Hydrochloride), Cyclophosphamide, Cytarabine, Daurismo (Glasdegib Maleate), Dexamethasone, Doxorubicin Hydrochloride, Enasidenib Mesylate, Gemtuzumab Ozogamicin, Gilteritinib Fumarate, Glasdegib Maleate, Idamycin PFS, Idarubicin, Idhifa, Ivosidenib, Midostaurin, Mitoxantrone Hydrochloride, Rydapt (Midostaurin), Thioguanine, Tibsovo (Ivosidenib), Venetoclax, and Vincristine Sulfate. However, the scope of chemotherapeutic agents contemplated herein which may be administered to a subject prior to being characterized and/or treated according to the methods as described herein are not limited, and other chemotherapeutic agents are envisaged. Such other chemotherapeutic agents are known in the art, and will be readily apparent to the skilled person. In some embodiments, a subject was administered a chemotherapeutic agent consisting of cytarabine (ara-C) prior to being characterized and/or treated according to the methods as described herein. In some embodiments, a subject was not administered a chemotherapeutic agent prior to being characterized and/or treated according to the methods as described herein.

In some embodiments, a subject (e.g., a human subject) has or is suspected of having a disease. A subject that “has or is suspected of having a disease” may exhibit one or more signs or symptoms of a particular disease (e.g., cancer), or may have been identified as having one or more genetic markers (e.g., genetic mutations, insertions, deletions, etc.) that increase the risk of the subject developing the disease (e.g., cancer). In some embodiments, the disease is related to a mutation in the genome of the subject, for example cancer resulting from the mutation of a cancer suppressor gene. In some embodiments, the disease is related to a chromosomal abnormality, such as a chromosomal substitution (e.g., mutation) or deletion, in the genome of the subject. In some embodiments, the subject is a subject having cancer. In some embodiments, the cancer is acute lymphoblastic leukemia (ALL), acute promyelocytic leukemia (APL), Chronic Myelogenous Leukemia (CML), or acute myeloid leukemia (AML). In some embodiments, the AML is pediatric AML. In some embodiments, the subject is less than 19 years of age. In some embodiments, the subject has one or more of: the presence of blast cells that express CD33 within the hematopoietic system; leukostasis; anemia; leukopenia; neutropenia; thrombocytopenia; chloroma; granulocytic sarcoma; and myeloid sarcoma.

The term “prognosis” refers to the prediction of the likelihood of death attributable to cancer or progression of cancer, including recurrence, metastatic spread, and drug resistance of a neoplastic disease, such as leukemia.

As used herein, “event free survival” and “EFS” refers to the length of time after primary treatment for a cancer ends (e.g., after primary treatment of a cancer ends) that the patient remains free of certain complications or events that the treatment was intended to prevent or delay, for example return of the cancer or onset of certain symptoms (e.g., bone pain from cancer that has spread to a bone). In some embodiments, a subject having a reduced likelihood of event free survival may have about a 1%, 5%, 10%, 20%, 50%, 75%, 90%, 95%, or 99% increased probability of recurrence of cancer relative to a subject that does not have a reduced likelihood of event free survival.

As used here, “overall survival” and “OS” refers to the length of time from either the date of diagnosis or the start of treatment for a disease, such as cancer, that patients diagnosed with the disease are still alive. A subject having a reduced likelihood of overall survival may have about a 1%, 5%, 10%, 20%, 50%, 75%, 90%, 95%, or 99% increased probability of dying prior to a subject that does not have a reduced likelihood of overall survival.

As used herein, “minimum residual disease” and “MRD” refer to small numbers of leukemic cells that remain in a subject during treatment, or after treatment, when the patient is in remission (e.g., has no symptoms or signs of disease). MRD testing is typically used to determine if a treatment has eradicated the cancerous cells (e.g., cancerous bone marrow cells) or whether small populations of cancerous cells remain. In some embodiments, MRD testing is used to detect recurrence of the leukemia in a subject. Generally detection of more than 1 cancerous cell out of 1,000 cells in a sample indicates a “high” MRD, or “MRD positive”, and a poor patient prognosis.

### Genes of Interest and Single Nucleotide Polymorphisms (SNPs)

The disclosure relates to the identification of certain genes comprising SNPs in the cytarabine (ara-C) metabolic pathway which, when analyzed according to the methods described herein, provide valuable information regarding cancer treatment outcomes.

Cytarabine (ara-C) is a deoxycytidine nucleoside analog useful in the treatment of certain cancers. As used herein, term “cytarabine”, “ara-C”, and “cytosine arabinoside” refer interchangeably to 1-(β-D-arabino-furanosyl)-cytosine and/or 4-amino-1-[(2R,3S,4R,5R)-3,4-dihydroxy-5-(hydroxymethyl)oxolan-2-yl]pyrimidin-2-one, and include all pharmaceutically acceptable salts, solvates, and prodrugs thereof, as well as combinations thereof. Once inside the cell, cytarabine (ara-C) phosphorylation by deoxycytidine kinase (DCK) is the rate-limiting step in its activation. The resulting cytarabine (ara-C) monophosphate (ara-CMP) is then further phosphorylated by pyrimidine kinases to the active 5′-triphosphate derivative, ara-cytidine-5′-triphosphate (ara-CTP). Conversely, the enzyme 5′-nucleotidase (NT5C2) can dephosphorylate ara-CMP back to cytarabine (ara-C). Cytarabine (ara-C) and ara-CMP can both be converted into the inactive forms, ara-U and ara-UMP, by the action of the enzymes cytidine deaminase (CDA) and deoxycytidylate deaminase (DCTD), respectively. DNA incorporation of ara-CTP in place of deoxycytidine triphosphate (dCTP) results in chain termination, blocking DNA and RNA synthesis and causing leukemic cell death, which, in turn, is associated with therapeutic response of cytarabine (ara-C). Several key candidate genes are known to be implicated in the metabolic activation of cytarabine (ara-C) to ara-cytidine-5′-triphosphate (ara-CTP), and include, inter alia, DCK, NT5C2, CDA, DCTD, SLC29A1, RRM1, and RRM2 (see, e.g., Lamba (2009) Genetic factors influencing cytarabine therapy, Pharmacogenomics, 10 (10): 1657-74). Other candidate genes involved in cytarabine metabolism may include CMPK1, NME4, CTPS1, and SLC28A3, as described herein.

A SNP is a variation in a single nucleotide in a nucleic acid sequence (e.g., DNA or mRNA) which is known to occur across a proportion of the population (>1% is a typical threshold to be considered a SNP; however, standards differ across the art). In DNA, two nucleotides will be present at each SNP, one on the positive strand of DNA, and one on the negative strand of DNA. As a non-limiting example of a SNP, a guanine (G) nucleotide might appear in a specific base position on the positive strand of DNA and an adenine (A) nucleotide might appear the same base position on the negative strand of DNA of a certain gene in most individuals (e.g., GA). However, in some individuals that same base position is occupied by an adenine (A) nucleotide on the positive strand of DNA and an adenine (A) nucleotide on the negative strand of DNA (e.g. AA).

In some embodiments, the genes comprising SNPs in the ara-C pathway comprise CDA, CMPK1, NME4, SLC29A1, RRM2, DCK, RRM1, CTPS1, and SLC28A3. In some embodiments, the genes comprising SNPs in the ara-C pathway are CDA, CMPK1, NME4, SLC29A1, RRM2, DCK, RRM1, CTPS1, and SLC28A3. In some embodiments, the SNPs in the foregoing genes comprise rs10916819 (CDA), rs17103168 (CMPK1), rs5841 (NME4), rs2396243 (SLC29A1), rs1044457 (CMPK1), rs1138729 (RRM2), rs4643786 (DCK), rs11030918 (RRM2), rs12067645 (CTPS1), and rs17343066 (SLC28A3). In some embodiments, the nucleotides present at each of a set of SNP locations comprising rs10916819, rs17103168, rs5841, rs2396243, rs1044457, rs1138729, rs4643786, rs11030918, rs12067645, and rs17343066 are identified. In some embodiments, a genotype score is assigned based upon the identified nucleotides present at each SNP, as described in detail below.

CDA (cytidine deaminase) is a protein coding gene which encodes an enzyme involved in pyrimidine salvaging. Mutations in this gene are associated with decreased sensitivity to the cytosine nucleoside analogue cytarabine, which is used in the treatment of certain childhood leukemias as described herein. In some embodiments, a CDA gene comprises a SNP at the chromosomal location corresponding to rs10916819. In some embodiments, the rs10916819 SNP comprises the substitution of a guanine (G) nucleotide in the base position which is typically occupied by an adenine (A) nucleotide (e.g., A>G). In some embodiments, the rs10916819 SNP comprises an adenine (A) nucleotide on the first strand of the CDA DNA, and an adenine (A) nucleotide on the second strand of the CDA DNA (e.g., AA). In some embodiments, the rs10916819 SNP comprising AA nucleotides is assigned a genotype score of zero (0). In some embodiments, the rs10916819 SNP comprises an adenine (A) nucleotide on the first strand of the CDA DNA, and a guanine (G) nucleotide on the second strand of the CDA DNA (e.g., AG). In some embodiments, the rs10916819 SNP comprising AG nucleotides is assigned a genotype score of negative one (−1). In some embodiments, the rs10916819 SNP comprises a guanine (G) nucleotide on the first strand of the CDA DNA, and a guanine (G) nucleotide on the second strand of the CDA DNA (e.g., GG). In some embodiments, the rs10916819 SNP comprising GG nucleotides is assigned a genotype score of negative one (−1).

CMPK1 (cytidine/uridine monophosphate kinase 1) is a protein coding gene which encodes one of the enzymes required for cellular nucleic acid biosynthesis. This enzyme catalyzes the transfer of a phosphate group from ATP to CMP, UMP, or dCMP, to form the corresponding diphosphate nucleotide. In some embodiments, a CMPK1 gene comprises a SNP at the chromosomal location corresponding to rs17103168. In some embodiments, the rs17103168 SNP comprises the substitution of a guanine (G) nucleotide in the base position which is typically occupied by an adenine (A) nucleotide (e.g., A>G). In some embodiments, the rs17103168 SNP comprises an adenine (A) nucleotide on the first strand of the CMPK1 DNA, and an adenine (A) nucleotide on the second strand of the CMPK1 DNA (e.g., AA). In some embodiments, the rs17103168 SNP comprising AA nucleotides is assigned a genotype score of zero (0). In some embodiments, the rs17103168 SNP comprises an adenine (A) nucleotide on the first strand of the CMPK1 DNA, and a guanine (G) nucleotide on the second strand of the CMPK1 DNA (e.g., AG). In some embodiments, the rs17103168 SNP comprising AG nucleotides is assigned a genotype score of one (1). In some embodiments, the rs17103168 SNP comprises a guanine (G) nucleotide on the first strand of the CMPK1 DNA, and a guanine (G) nucleotide on the second strand of the CMPK1 DNA (e.g., GG). In some embodiments, the rs17103168 SNP comprising GG nucleotides is assigned a genotype score of one (1). In some embodiments, a CMPK1 gene comprises a SNP at the chromosomal location corresponding to rs1044457. In some embodiments, the rs1044457 SNP comprises the substitution of a thymine (T) nucleotide in the base position which is typically occupied by a cytosine (C) nucleotide (e.g., C>T). In some embodiments, the rs1044457 SNP comprises a cytosine (C) nucleotide on the first strand of the CMPK1 DNA, and a cytosine (C) nucleotide on the second strand of the CMPK1 DNA (e.g., CC). In some embodiments, the rs1044457 SNP comprising CC nucleotides is assigned a genotype score of zero (0). In some embodiments, the rs1044457 SNP comprises a cytosine (C) nucleotide on the first strand of the CMPK1 DNA, and a thymine (T) nucleotide on the second strand of the CMPK1 DNA (e.g., CT). In some embodiments, the rs1044457 SNP comprising CT nucleotides is assigned a genotype score of one (1). In some embodiments, the rs1044457 SNP comprises a thymine (T) nucleotide on the first strand of the CMPK1 DNA, and a thymine (T) nucleotide on the second strand of the CMPK1 DNA (e.g., TT). In some embodiments, the rs1044457 SNP comprising TT nucleotides is assigned a genotype score of one (1).

NME4 (nucleoside diphosphate kinase 4) is a protein coding gene which encodes an enzyme that catalyzes transfer of gamma-phosphates, via a phosphohistidine intermediate, between nucleoside and dioxynucleoside tri- and diphosphates. In some embodiments, a NME4 gene comprises a SNP at the chromosomal location corresponding to rs5841. In some embodiments, the rs5841 SNP comprises the substitution of a thymine (T) nucleotide in the base position which is typically occupied by a cytosine (C) nucleotide (e.g., C>T). In some embodiments, the rs5841 SNP comprises a cytosine (C) nucleotide on the first strand of the NME4 DNA, and a cytosine (C) nucleotide on the second strand of the NME4 DNA (e.g., CC). In some embodiments, the rs5841 SNP comprising CC nucleotides is assigned a genotype score of zero (0). In some embodiments, the rs5841 SNP comprises a cytosine (C) nucleotide on the first strand of the NME4 DNA, and a thymine (T) nucleotide on the second strand of the NME4 DNA (e.g., CT). In some embodiments, the rs5841 SNP comprising CT nucleotides is assigned a genotype score of one (1). In some embodiments, the rs5841 SNP comprises a thymine (T) nucleotide on the first strand of the NME4 DNA, and a thymine (T) nucleotide on the second strand of the NME4 DNA (e.g., TT). In some embodiments, the rs5841 SNP comprising TT nucleotides is assigned a genotype score of one (1).

SLC29A1 (solute carrier family 29 member 1) is a protein coding gene which encodes a transmembrane glycoprotein that localizes to the plasma and mitochondrial membranes and mediates the cellular uptake of nucleosides from the surrounding medium. Nucleoside transporters are required for nucleotide synthesis in cells that lack de novo nucleoside synthesis pathways, and are also necessary for the uptake of cytotoxic nucleosides used for cancer and viral chemotherapies. In some embodiments, a SLC29A1 gene comprises a SNP at the chromosomal location corresponding to rs2396243. In some embodiments, the rs2396243 SNP comprises the substitution of an adenine (A) nucleotide in the base position which is typically occupied by a guanine (G) nucleotide (e.g., G>A). In some embodiments, the rs2396243 SNP comprises a guanine (G) nucleotide on the first strand of the SLC29AI DNA, and a guanine (G) nucleotide on the second strand of the SLC29AI DNA (e.g., GG). In some embodiments, the rs2396243 SNP comprising GG nucleotides is assigned a genotype score of zero (0). In some embodiments, the rs2396243 SNP comprises an adenine (A) nucleotide on the first strand of the SLC29AI DNA, and a guanine (G) nucleotide on the second strand of the SLC29AI DNA (e.g., AG). In some embodiments, the rs2396243 SNP comprising AG nucleotides is assigned a genotype score of negative one (−1). In some embodiments, the rs2396243 SNP comprises an adenine (A) nucleotide on the first strand of the SLC29AI DNA, and an adenine (A) nucleotide on the second strand of the SLC29AI DNA (e.g., AA). In some embodiments, the rs2396243 SNP comprising AA nucleotides is assigned a genotype score of negative two (−2).

RRM2 (ribonucleotide reductase regulatory subunit M2) is a protein coding gene which encodes one of two non-identical subunits for ribonucleotide reductase. This reductase catalyzes the formation of deoxyribonucleotides from ribonucleotides. In some embodiments, a RRM2 gene comprises a SNP at the chromosomal location corresponding to rs1138729. In some embodiments, the rs1138729 SNP comprises the substitution of a guanine (G) nucleotide in the base position which is typically occupied by an adenine (A) nucleotide (e.g., A>G). In some embodiments, the rs1138729 SNP comprises an adenine (A) nucleotide on the first strand of the RRM2 DNA, and an adenine (A) nucleotide on the second strand of the RRM2 DNA (e.g., AA). In some embodiments, the rs1138729 SNP comprising AA nucleotides is assigned a genotype score of zero (0). In some embodiments, the rs1138729 SNP comprises an adenine (A) nucleotide on the first strand of the RRM2 DNA, and a guanine (G) nucleotide on the second strand of the RRM2 DNA (e.g., AG). In some embodiments, the rs1138729 SNP comprising AG nucleotides is assigned a genotype score of negative one (−1). In some embodiments, the rs1138729 SNP comprises a guanine (G) nucleotide on the first strand of the RRM2 DNA, and a guanine (G) nucleotide on the second strand of the RRM2 DNA (e.g., GG). In some embodiments, the rs1138729 SNP comprising GG nucleotides is assigned a genotype score of negative one (−1).

DCK (deoxycytidine kinase) is a protein coding gene which encodes the enzyme deoxycytidine kinase. Deoxycytidine kinase is required for the phosphorylation of several deoxyribonucleosides and their nucleoside analogs. Deficiency of DCK is associated with resistance to antiviral and anticancer chemotherapeutic agents. Conversely, increased deoxycytidine kinase activity is associated with increased activation of these compounds to cytotoxic nucleoside triphosphate derivatives. In some embodiments, a DCK gene comprises a SNP at the chromosomal location corresponding to rs4643786. In some embodiments, the rs4643786 SNP comprises the substitution of a cytosine (C) nucleotide in the base position which is typically occupied by a thymine (T) nucleotide (e.g., T>C). In some embodiments, the rs4643786 SNP comprises a thymine (T) nucleotide on the first strand of the DCK DNA, and a thymine (T) nucleotide on the second strand of the DCK DNA (e.g., TT). In some embodiments, the rs4643786 SNP comprising TT nucleotides is assigned a genotype score of zero (0). In some embodiments, the rs4643786 SNP comprises a cytosine (C) nucleotide on the first strand of the DCK DNA, and a thymine (T) nucleotide on the second strand of the DCK DNA (e.g., CT). In some embodiments, the rs4643786 SNP comprising CT nucleotides is assigned a genotype score of negative one (−1). In some embodiments, the rs4643786 SNP comprises a cytosine (C) nucleotide on the first strand of the DCK DNA, and a cytosine (C) nucleotide on the second strand of the DCK DNA (e.g., CC). In some embodiments, the rs4643786 SNP comprising CC nucleotides is assigned a genotype score of negative two (−2).

RRM1 (ribonucleotide reductase regulatory subunit M1) is a protein coding gene which encodes the large and catalytic subunit of ribonucleotide reductase, an enzyme essential for the conversion of ribonucleotides into deoxyribonucleotides. A pool of available deoxyribonucleotides is important for DNA replication during S phase of the cell cycle as well as multiple DNA repair processes. In some embodiments, a RRM1 gene comprises a SNP at the chromosomal location corresponding to rs11030918. In some embodiments, the rs11030918 SNP comprises the substitution of a cytosine (C) nucleotide in the base position which is typically occupied by a thymine (T) nucleotide (e.g., T>C). In some embodiments, the rs11030918 SNP comprises a thymine (T) nucleotide on the first strand of the RRM1 DNA, and a thymine (T) nucleotide on the second strand of the RRM1 DNA (e.g., TT). In some embodiments, the rs11030918 SNP comprising TT nucleotides is assigned a genotype score of zero (0). In some embodiments, the rs11030918 SNP comprises a cytosine (C) nucleotide on the first strand of the RRM1 DNA, and a thymine (T) nucleotide on the second strand of the RRM1 DNA (e.g., CT). In some embodiments, the rs11030918 SNP comprising CT nucleotides is assigned a genotype score of zero (0). In some embodiments, the rs11030918 SNP comprises a cytosine (C) nucleotide on the first strand of the RRM1 DNA, and a cytosine (C) nucleotide on the second strand of the RRM1 DNA (e.g., CC). In some embodiments, the rs11030918 SNP comprising CC nucleotides is assigned a genotype score of one (1).

CTPS1 (CTP synthase 1) is a protein coding gene which encodes an enzyme responsible for the catalytic conversion of UTP (uridine triphosphate) to CTP (cytidine triphospate). This reaction is an important step in the biosynthesis of phospholipids and nucleic acids. Activity of this protein is important in the immune system, and loss of function of this gene has been associated with immunodeficiency. In some embodiments, a CTPS1 gene comprises a SNP at the chromosomal location corresponding to rs12067645. In some embodiments, the rs12067645 SNP comprises the substitution of an adenine (A) nucleotide in the base position which is typically occupied by a guanine (G) nucleotide (e.g., G>A). In some embodiments, the rs12067645 SNP comprises a guanine (G) nucleotide on the first strand of the CTPS1 DNA, and a guanine (G) nucleotide on the second strand of the CTPS1 DNA (e.g., GG). In some embodiments, the rs12067645 SNP comprising GG nucleotides is assigned a genotype score of zero (0). In some embodiments, the rs12067645 SNP comprises an adenine (A) nucleotide on the first strand of the CTPS1 DNA, and a guanine (G) nucleotide on the second strand of the CTPS1 DNA (e.g., AG). In some embodiments, the rs12067645 SNP comprising AG nucleotides is assigned a genotype score of one (1). In some embodiments, the rs12067645 SNP comprises an adenine (A) nucleotide on the first strand of the CTPS1 DNA, and an adenine (A) nucleotide on the second strand of the CTPS1 DNA (e.g., AA). In some embodiments, the rs12067645 SNP comprising AA nucleotides is assigned a genotype score of two (2).

SLC28A3 (solute carrier family 28 member 3) is a protein coding gene which encodes the nucleoside transporter SLC28A3. Nucleoside transporters, such as SLC28A3, regulate multiple cellular processes, including neurotransmission, vascular tone, adenosine concentration in the vicinity of cell surface receptors, and transport and metabolism of nucleoside drugs. In some embodiments, a SLC28A3 gene comprises a SNP at the chromosomal location corresponding to rs17343066. In some embodiments, the rs17343066 SNP comprises the substitution of an adenine (A) nucleotide in the base position which is typically occupied by a guanine (G) nucleotide (e.g., G>A). In some embodiments, the rs17343066 SNP comprises a guanine (G) nucleotide on the first strand of the SLC28A3 DNA, and a guanine (G) nucleotide on the second strand of the SLC28A3 DNA (e.g., GG). In some embodiments, the rs17343066 SNP comprising GG nucleotides is assigned a genotype score of zero (0). In some embodiments, the rs17343066 SNP comprises an adenine (A) nucleotide on the first strand of the SLC28A3 DNA, and a guanine (G) nucleotide on the second strand of the SLC28A3 DNA (e.g., AG). In some embodiments, the rs17343066 SNP comprising AG nucleotides is assigned a genotype score of zero (0). In some embodiments, the rs17343066 SNP comprises an adenine (A) nucleotide on the first strand of the SLC28A3 DNA, and an adenine (A) nucleotide on the second strand of the SLC28A3 DNA (e.g., AA). In some embodiments, the rs17343066 SNP comprising AA nucleotides is assigned a genotype score of one (1).

As will be understood, in some instances the identified SNPs described herein (e.g., rs10916819, rs17103168, rs5841, rs2396243, rs1044457, rs1138729, rs4643786, rs11030918, rs12067645, and rs17343066) may occur in linkage disequilibrium. Linkage disequilibrium is the non-random association of alleles at different loci in a given population (for a review on linkage disequilibrium, see Slatkin (2008), Linkage disequilibrium—understanding the evolutionary past and mapping the medical future, Nat Rev Genet 9, 477-85). In instances of linkage disequilibrium, other SNPs may, in some embodiments, be used as surrogates of the identified SNPs described herein (e.g., surrogates of each of rs10916819, rs17103168, rs5841, rs2396243, rs1044457, rs1138729, rs4643786, rs11030918, rs12067645, and rs17343066). Surrogate SNPs for each identified SNP are listed in Table 1.

In some embodiments, the nucleotides present at each of a set of SNP locations comprising a SNP surrogate of rs10916819, a SNP surrogate of rs17103168, a SNP surrogate of rs5841, a SNP surrogate of rs2396243, a SNP surrogate of rs1044457, a SNP surrogate of rs1138729, a SNP surrogate of rs4643786, a SNP surrogate of rs11030918, a SNP surrogate of rs12067645, and a SNP surrogate of rs17343066, as shown in Table 1, are identified. In some embodiments, a genotype score is assigned based upon the identified nucleotides present at each SNP surrogate, as described in detail above.

TABLE 1
Surrogate SNPs for calculating ACS10 score in linkage disequilibrium
SNPs occurring in LD (linkage disequilibrium) with CDA SNP rs10916819:

| | | | | | |
|---|---|---|---|---|---|
| s10799640 | rs4399127 | rs4529682 | rs4291476 | rs4571937 | rs4439331 |
| rs7416238 | rs10799641 | rs10916817 | rs10916818 | rs10737450 | rs10799642 |
| rs6701759 | rs6691041 | rs1890006 | rs1890005 | rs1890004 | rs1890003 |
| rs4655221 | rs10737451 | rs4655222 | rs4655223 | rs4655224 | rs79985745 |
| rs4655225 | rs7545313 | rs7524492 | rs10799643 | rs7547998 | rs10799644 |
| rs12409100 | rs12059125 | rs12059126 | rs12089623 | rs10799645 | rs10799646 |
| rs12023637 | rs12042071 | rs12044525 | rs10916821 | rs2934358 | rs6704070 |

SNPs occurring in LD (linkage disequilibrium) with CMPK1 SNP rs17103168:

| | | | | | |
|---|---|---|---|---|---|
| rs12729251 | rs12184297 | rs12184269 | rs12184262 | rs67905093 | rs11587014 |
| rs11583162 | rs11587107 | rs12406951 | rs72686209 | rs12131978 | rs12129594 |
| rs12132909 | rs12410850 | rs17103095 | rs12407463 | rs60248293 | rs56804458 |
| rs11211512 | rs34216828 | rs6660651 | rs35601384 | rs12139700 | rs11576796 |
| rs11577236 | rs11587272 | rs12751618 | rs12145103 | rs4481897 | rs7415976 |
| rs11211516 | rs9725517 | rs60352530 | rs12123269 | rs67221389 | rs12117686 |
| rs10157734 | rs12134888 | rs12135037 | rs67295054 | rs12132521 | rs12749507 |
| rs59685566 | rs11579854 | rs34979288 | rs34192192 | rs2622914 | rs7534328 |
| rs12718447 | rs12129259 | rs2820986 | rs2622903 | rs6698471 | rs6698717 |
| rs4926840 | rs201573193 | rs199585608 | rs12757893 | rs11584518 | rs17370386 |
| rs149781289 | rs11211526 | rs11586975 | rs12144920 | rs35856603 | rs11211495 |
| rs13376679 | rs58891696 | rs11211500 | rs2897238 | rs12118205 | rs7354822 |
| rs11577960 | rs199580672 | rs35234940 | rs12756634 | rs11211489 | rs11211490 |
| rs11211492 | rs12123358 | rs12143808 | rs13374382 | rs33980430 | rs2405787 |
| rs139519885 | rs12118206 | rs12738065 | rs11211505 | rs12145974 | rs6688804 |
| rs7548099 | rs11580114 | rs11578360 | rs11580148 | rs142647386 | rs17378887 |
| rs36054263 | rs12759377 | rs7528075 | rs12065137 | rs78431586 | rs11211533 |
| rs12751405 | rs201981799 | rs7525802 | rs4480403 | rs12080109 | rs10890480 |
| rs6691723 | rs12735320 | rs1572546 | | | |

SNPs occurring in LD (linkage disequilibrium) with NME4 rs5841:

| | | | | | |
|---|---|---|---|---|---|
| rs7185254 | rs2363759 | rs763151 | rs14293 | rs11863625 | rs28394836 |
| rs448063 | | | | | |

SNPs occurring in LD (linkage disequilibrium) with SLC29A1 rs2396243:

| | | | | | |
|---|---|---|---|---|---|
| rs7773444 | rs6458373 | rs9472232 | rs6458374 | rs3734701 | rs693955 |
| rs1999416 | | | | | |

SNPs occurring in LD (linkage disequilibrium) with CMPK1 rs1044457:

| | | | | | |
|---|---|---|---|---|---|
| rs12141060 | rs6703956 | rs6692734 | rs35711761 | rs12138072 | rs12732050 |
| rs17452995 | rs34400183 | rs12126139 | rs12118514 | rs4926824 | rs6681868 |
| rs34952704 | rs3125639 | rs3125640 | rs12121831 | rs12562561 | rs55737131 |
| rs35072721 | rs2820994 | rs2622925 | rs12127982 | rs2820989 | rs2622908 |
| rs2820988 | rs2622907 | rs6588365 | rs2622905 | rs2246707 | rs2820987 |
| rs3122610 | rs2622901 | rs66485259 | rs6795 | rs7523987 | rs72686249 |
| rs17370337 | rs7533562 | rs12731806 | rs2622926 | rs55918644 | rs56328227 |
| rs12730819 | rs55711287 | rs35774681 | rs61782706 | rs12732410 | rs12125261 |
| rs3122623 | rs199860855 | rs12743643 | rs71645861 | rs56391477 | rs12119783 |
| rs56270164 | rs10437097 | | | | |

SNPs occurring in LD (linkage disequilibrium) with RRM2 rs1138729:

| | | | | | |
|---|---|---|---|---|---|
| rs72544298 | rs113313134 | rs13395772 | rs4669534 | rs4669536 | rs11885066 |
| rs4669537 | rs112860672 | | | | |
| rs6432063 | rs7574663 | rs112311121 | rs11691833 | rs111811121 | rs200931993 |
| rs4669529 | rs141200216 | rs7585544 | rs148756186 | rs73167392 | rs7582263 |

SNPs occurring in LD (linkage disequilibrium) with RRM1 rs11030918:

| | | | | | |
|---|---|---|---|---|---|
| rs12806698 | rs10835611 | rs7111104 | rs7932702 | rs7940013 | rs2268168 |
| rs2056853 | rs3817657 | rs4910886 | rs4910887 | rs35359151 | rs12290828 |
| rs2165728 | rs2284450 | rs4910888 | rs4910889 | rs4910599 | rs10742242 |
| rs10767845 | rs9666229 | rs9666857 | rs11031110 | rs10835556 | rs10835557 |
| rs11030781 | | | | | |

SNPs occurring in LD (linkage disequilibrium) with DCK rs4643786:

| | | | | | |
|---|---|---|---|---|---|
| rs7684954 | rs72854116 | rs28668057 | rs28668618 | rs28508004 | rs6829835 |
| rs6446979 | rs6446982 | rs3775289 | rs9993633 | rs6446988 | rs114734420 |
| rs151338753 | rs2129173 | rs10805074 | rs7684954 | rs4235090 | rs4490428 |
| rs6446998 | rs6841923 | rs201769235 | rs10518080 | rs11935198 | rs7439562 |
| rs28734949 | rs936869 | rs936868 | rs1486271 | rs1385986 | rs4525938 |
| rs11947877 | rs11935167 | rs12498308 | rs10938129 | rs4308342 | rs60861707 |

SNPs occurring in LD (linkage disequilibrium) with RRMI rs12067645:

| | | | | | |
|---|---|---|---|---|---|
| rs11580558 | rs11578829 | rs28570583 | rs28582097 | rs28397033 | rs28641158 |
| rs28433989 | rs28558724 | rs72948869 | rs7530304 | rs76179192 | rs79425393 |
| rs11579890 | rs12736893 | rs11578762 | rs11582760 | rs12405668 | rs72663705 |
| rs67325189 | rs7529969 | rs7524587 | rs6670153 | rs6668138 | rs12062753 |
| rs7514344 | rs6676944 | rs12065768 | rs12066993 | rs76143390 | rs80295497 |
| rs12407403 | rs12404862 | rs12404876 | rs6669459 | rs6687286 | |

SNPs occurring in LD (linkage disequilibrium) with SLC28A3 rs17343066:

| | | | | | |
|---|---|---|---|---|---|
| rs11342897 | rs4588940 | rs4242627 | rs4242628 | rs58075154 | rs57409783 |
| rs4877847 | | | | | |

In some embodiments, the methods of the disclosure further comprise performing an assay to detect the genotype of the subject for the SNP rs12459419, which is comprised in the CD33 gene. In such embodiments, a genotype score is not assigned; rather, the subject is characterized and/or treated based only on the identity of the nucleotides present at the rs12459419 SNP. CD33 is a protein coding gene which encodes the CD33 molecule, which is an inhibitory receptor with differential ITIM function in recruiting the phosphatases SHP-1 and SHP-2. Diseases associated with CD33 include Acute Leukemia and Acute Promyelocytic Leukemia. In some embodiments, the rs12459419 SNP comprises a cytosine (C) nucleotide on the first strand of the CD33 DNA, and a cytosine (C) nucleotide on the second strand of the CD33 DNA (e.g., CC). In some embodiments, the rs12459419 SNP comprises a thymine (T) nucleotide on the first strand of the CD33 DNA, and a cytosine (C) nucleotide on the second strand of the CD33 DNA (e.g., TC). In some embodiments, the rs12459419 SNP comprises a thymine (T) nucleotide on the first strand of the CD33 DNA, and a thymine (T) nucleotide on the second strand of the CD33 DNA (e.g., TT). In some embodiments, a therapeutically effective amount of an agent that selectively binds to CD33 when the subject exhibits a CC genotype for the rs12459419 SNP. Such agents are described elsewhere herein.

### Calculating ACS10 Scores

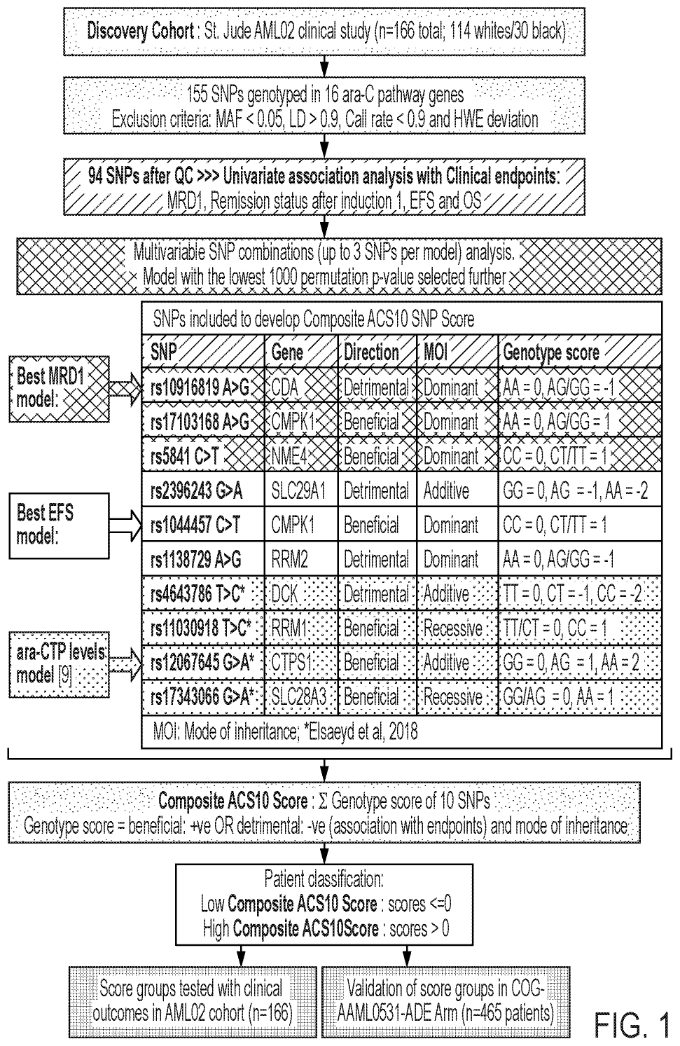

Aspects of the disclosure relate to characterizing and/or treating a subject having cancer based upon on the summation of the assigned genotype scores (e.g., the ACS10 score) for a set of SNPs, said assigned genotype scores being assigned according to the methods set forth above. In some embodiments, the summation of the assigned genotype scores is calculated by adding the genotype scores for each SNP, said assigned genotype scores being assigned according to the methods set forth above (see FIG. 1).

As described elsewhere herein, in some embodiments genotype scores are assigned following the performance of an assay to genotype a subject having cancer for certain SNPs of interest. Accordingly, in some embodiments, the presently described methods comprise (i) performing an assay to genotype a subject having cancer for certain SNPs of interest, (ii) assigning genotype scores for each SNP, and (iii) calculating the summation of the assigned genotype scores. However, methods which do not comprise performing an assay to genotype a subject having cancer for certain SNPs of interest are also specifically contemplated herein. In some embodiments, an assay is performed to genotype a SNP(s) of interest prior to practicing the methods of the present disclosure. The user who performs the genotyping assay is not the same user who performs the presently described methods of characterizing and/or treating a subject having cancer, in some embodiments. In such embodiments, the methods described herein comprise characterizing and/or treating a subject having cancer based upon the SNP genotype data previously obtained, and do not comprise performing an assay to genotype a SNP(s) of interest, as described herein. In some embodiments, the genotype score is assigned based on information previously obtained from the sample (e.g., the assay has already been performed).

Provided here is a non-limiting example of calculating an ACS10 score based on the following hypothetical SNP genotypes in a single subject having cancer:

| SNP location | Characterized nucleotides | Assigned genotype score |
|---|---|---|
| rs10916819 | AA | 0 |
| rs17103168 | AG | 1 |
| rs5841 | CT | 1 |
| rs2396243 | GG | 0 |
| rs1044457 | CC | 0 |
| rs1138729 | GG | −1 |
| rs4643786 | CC | −2 |
| rs11030918 | TT | 0 |
| rs12067645 | AG | 1 |
| rs17343066 | AA | 1 |

Summation of assigned genotype scores (ACS10 score) = 1

In some embodiments, the subject is characterized and/or treated based on the summation of the assigned genotype scores (e.g., the ACS10 score) being categorized as (a) less than or equal to zero (0) (e.g., 0, −1, −2, −3, −4, −5) or (b) greater than zero (0) (e.g., 1, 2, 3, 4, 5, 6, 7).

In some embodiments, the summation of assigned genotype scores (e.g., the ACS10 score) is less than or equal to zero (0) (e.g., 0, −1, −2, −3, −4, −5). In some embodiments, an ACS10 score which is less than or equal to zero (0) (e.g., 0, −1, −2, −3, −4, −5) indicates that the subject is likely to benefit from administration of a high dose of cytarabine, as described elsewhere herein. In some embodiments, an ACS10 score which is less than or equal to zero (0) (e.g., 0, −1, −2, −3, −4, −5) indicates that the subject is likely to benefit from administration of a therapeutically effective amount of an agent that selectively binds to CD33, as described elsewhere herein.

In some embodiments, the summation of assigned genotype scores (e.g., the ACS10 score) is greater than zero (0) (e.g., 1, 2, 3, 4, 5, 6, 7). In some embodiments, an ACS10 score which is greater than zero (0) (e.g., 1, 2, 3, 4, 5, 6, 7) indicates that the subject is not likely to benefit from administration of a high dose of cytarabine, as described elsewhere herein, and/or that administration of a high dose of cytarabine may result in negative outcomes for the subject. In some embodiments, an ACS10 score which is greater than zero (0) (e.g., 1, 2, 3, 4, 5, 6, 7) indicates that the subject is likely to benefit from administration of a low dose of cytarabine, as described elsewhere herein.

### Therapeutic Methods

Aspects of the disclosure relate to methods of treating a subject having cancer, wherein the methods comprise administering a particular therapeutic agent or agents based on the characterization of certain SNP genotypes in the cytarabine (ara-C) pathway, as described herein.

In some embodiments, methods of characterizing a subject as described herein further comprise a step of administering a chemotherapeutic agent to the subject after the characterizing. In some embodiments, the chemotherapeutic agents comprise cytarabine (ara-C), daunorubicin, etoposide, or the combination of these drugs—which is referred to as “ADE”. Other examples of chemotherapeutic agents include, but are not limited to, Arsenic Trioxide, Cerubidine (Daunorubicin Hydrochloride), Cyclophosphamide, Cytarabine, Daurismo (Glasdegib Maleate), Dexamethasone, Doxorubicin Hydrochloride, Enasidenib Mesylate, Gemtuzumab Ozogamicin, Gilteritinib Fumarate, Glasdegib Maleate, Idamycin PFS, Idarubicin, Idhifa, Ivosidenib, Midostaurin, Mitoxantrone Hydrochloride, Rydapt (Midostaurin), Thioguanine, Tibsovo (Ivosidenib), Venetoclax, and Vincristine Sulfate. However, the scope of chemotherapeutic agents contemplated herein which may be administered to a subject prior to being characterized and/or treated according to the methods as described herein are not limited, and other chemotherapeutic agents are envisaged. Such other chemotherapeutic agents are known in the art, and will be readily apparent to the skilled person.

In some embodiments, the subject is administered cytarabine at a high dose when the summation of the assigned genotype scores (e.g., the ACS10 score), calculated as described herein, is less than or equal to zero (0) (e.g., 0, −1, −2, −3, −4, −5). A “high” dose of cytarabine (HDAraC, or HDAC) is an art-recognized dosage which generally comprises between about 2 g/m² and 3 g/m², administered twice daily (e.g., every twelve (12) hours), with 3 g/m² every twelve (12) hours being a common HDAC (see, for example, Wu, et al., (2017) Efficacy and safety of different doses of cytarabine in consolidation therapy for adult acute myeloid leukemia patients: a network meta-analysis, Sci Rep 7:9509; Baer, et al., (1993) High-dose cytarabine, idarubicin, and granulocyte colony-stimulating factor remission induction therapy for previously untreated de novo and secondary adult acute myeloid leukemia, Semin Oncol 20 (6 Suppl 8): 6-12). In some embodiments, a high dose of cytarabine comprises about 1.5 g/m², about 1.6 g/m², about 1.7 g/m², about 1.8 g/m², about 1.9 g/m², about 2.0 g/m², about 2.1 g/m², about 2.2 g/m², about 2.3 g/m², about 2.4 g/m², about 2.5 g/m², about 2.6 g/m², about 2.7 g/m², about 2.8 g/m², about 2.9 g/m², about 3.0 g/m², about 3.1 g/m², about 3.2 g/m², about 3.3 g/m², about 3.4 g/m², or about 3.5 g/m², administered twice daily (e.g., every twelve (12) hours). In some embodiments, a high dose of cytarabine comprises a dose in a range of about 1.5 g/m² to about 1.7 g/m², about 1.6 g/m² to about 1.9 g/m², about 1.8 g/m² to about 2.1 g/m², about 2.0 g/m² to about 2.3 g/m², about 2.2 g/m² to about 2.5 g/m², about 2.4 g/m² to about 2.7 g/m², about 2.6 g/m² to about 2.9 g/m², about 2.8 g/m² to about 3.1 g/m², or about 3.1 g/m² to about 3.5 g/m², administered twice daily (e.g., every twelve (12) hours). In some embodiments, a high dose of cytarabine is 2 g/m², administered twice daily (e.g., every twelve (12) hours). In some embodiments, a high dose of cytarabine is 3 g/m², administered twice daily (e.g., every twelve (12) hours).

As will be understood, a HDAC may also comprise about 1.5 g/m² for patients over a certain age, for example patients over the age of 55. In some embodiments, wherein the subject is over the age of 55, a high dose of cytarabine comprises about 1.0 g/m², about 1.1 g/m², about 1.2 g/m², about 1.3 g/m², about 1.4 g/m², about 1.5 g/m², about 1.6 g/m², about 1.7 g/m², about 1.8 g/m², about 1.9 g/m², about 2.0 g/m², about 2.1 g/m², about 2.2 g/m², about 2.3 g/m², about 2.4 g/m², about 2.5 g/m², about 2.6 g/m², about 2.7 g/m², about 2.8 g/m², about 2.9 g/m², about 3.0 g/m², about 3.1 g/m², about 3.2 g/m², about 3.3 g/m², about 3.4 g/m², or about 3.5 g/m², administered twice daily (e.g., every twelve (12) hours). In some embodiments, wherein the subject is over the age of 55, a high dose of cytarabine comprises a dose in a range of about 1.0 g/m² to about 1.3 g/m², about 1.2 g/m² to about 1.5 g/m², about 1.4 g/m² to about 1.7 g/m², about 1.6 g/m² to about 1.9 g/m², about 1.8 g/m² to about 2.1 g/m², about 2.0 g/m² to about 2.3 g/m², about 2.2 g/m² to about 2.5 g/m², about 2.4 g/m² to about 2.7 g/m², about 2.6 g/m² to about 2.9 g/m², about 2.8 g/m² to about 3.1 g/m², or about 3.1 g/m² to about 3.5 g/m², administered twice daily (e.g., every twelve (12) hours). In some embodiments, wherein the subject is over the age of 55, a high dose of cytarabine is 1.5 g/m², administered twice daily (e.g., every twelve (12) hours).

In some embodiments, the subject is further administered an agent (e.g., in addition to a low dose of cytarabine) that selectively binds to CD33 when the summation of the assigned genotype scores (e.g., the ACS10 score), calculated as described herein, is less than or equal to zero (0) (e.g., 0, −1, −2, −3, −4, −5). Alternatively or additionally, in some embodiments the subject is further administered an agent (e.g., in addition to a low dose of cytarabine) that selectively binds to CD33 when the subject exhibits a CC genotype for the CD33 single-nucleotide polymorphism rs12459419, as described elsewhere herein and in International Publication Number WO 2017/177011, incorporated by reference herein in its entirety.

In some embodiments, the agent that selectively binds to CD33 is gemtuzumab ozogamicin (GO), hP67.7, SGN-33A, or an antibody that selectively binds CD33 or an antigen binding fragment thereof. In some embodiments, the agent that selectively binds to CD33 is gemtuzumab ozogamicin (GO). In some embodiments, GO is administered to the subject of a dose of about 3 mg/m² or about 6 mg/m². In some embodiments, GO is administered to the subject of a dose of about 1 mg/m², about 2 mg/m², about 3 mg/m², about 4 mg/m², about 5 mg/m², about 6 mg/m², about 7 mg/m², about 8 mg/m², about 9 mg/m², or about 10 mg/m². In some embodiments, GO is administered to the subject of a dose in a range of about 1 mg/m² to about 3 mg/m², about 2 mg/m² to about 4 mg/m², about 3 mg/m² to about 5 mg/m², about 4 mg/m² to about 6 mg/m², about 5 mg/m² to about 7 mg/m², about 6 mg/m² to about 8 mg/m², about 7 mg/m² to about 9 mg/m², or about 8 mg/m² to about 10 mg/m². In some embodiments, GO is administered to the subject of a dose of 3 mg/m². In some embodiments, GO is administered to the subject of a dose of 6 mg/m².

In some embodiments, the agent that selectively binds to CD33 is an antibody, or antigen binding fragment thereof. Any antibody that selectively binds CD33 may be used. The term antibody is used in the broadest sense and specifically includes, for example, single monoclonal antibodies, antibody compositions with polyepitopic specificity, single chain antibodies, and antigen-binding fragments of antibodies. An antibody may include an immunoglobulin constant domain from any immunoglobulin, such as IgG1, IgG2, IgG3, or IgG4 subtypes, IgA (including IgA1 and IgA2), IgE, IgD, or IgM. As used herein, an antigen-binding fragment refers to a portion of an intact antibody that binds antigen. Examples of antibody fragments include Fab, Fab′, F(ab′)₂, and Fv fragments; diabodies; linear antibodies (Zapata et al., Protein Eng. 8 (10): 1057-1062 [1995]); and single-chain antibody molecules. Fv is the minimum antibody fragment containing a complete antigen-recognition binding site. This region consists of a dimer of one heavy- and one light-chain variable domain in tight, non-covalent association. In this configuration the three CDRs of each variable domain interact to define an antigen-binding site on the surface of the V_{H}-V_{L} dimer. Collectively, the six CDRs confer antigen-binding specificity to the antibody. The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. F(ab′)₂ antibody fragments originally were produced as pairs of Fab′ fragments which have hinge cysteines between them. Other chemical couplings of antibody fragments are also known. In some embodiments, the antibody is a full length antibody (i.e., contains an Fc region, which can be IgG4 for example).

In some embodiments, the agent that selectively binds to CD33 is a humanized antibody. Humanized forms of non-human (e.g., murine) antibodies are chimeric immunoglobulins (including full length immunoglobulins), immunoglobulin chains or fragments thereof (such as Fv, Fab, Fab′, F(ab′) 2, scFv or other antigen-binding subsequences of antibodies) which contain minimal sequence derived from the non-human immunoglobulin. Humanized antibodies typically include human immunoglobulins (recipient antibody) in which residues from a complementary determining region (CDR) of the recipient are replaced by residues from a CDR of a non-human species (donor antibody) such as mouse, rat or rabbit having the desired specificity, affinity, and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Further details respecting antibodies and general methods of making antibodies can be found in U.S. patent application publication number 2013/0136735, the entire disclosure of which is incorporated herein by reference.

The antibodies selectively bind their targets, such as CD33 on blast cells. An antibody that selectively binds its target cell(s) means it has the ability to be used in vitro or in vivo to bind to and distinguish such target bearing tissue from other tissue types of the species, including other closely related cell types under the conditions in which the antibody is used, such as under physiologic conditions. In some embodiments, the antibody selectively binds human blast cells that express CD33. In some embodiments, the antibody selectively binds to any region of CD33. In some embodiments, the antibody selectively binds to the IgV domain of CD33. In some embodiments, the antibody is GO. In some embodiments, the antibody is SGN-CD33A. In some embodiments, the antibody is hP67.7. In some embodiments, the antibody is hP67.7 linked to a toxin. The antibody can be any antibody or antigen binding fragment thereof that selectively binds CD33 and is linked to a toxin.

Aspects of the invention relate to treatment with an antibody drug conjugate (ADC), such as an antibody or antigen binding fragment thereof that selectively binds to CD33, which is directly linked to a toxin or linked to a toxin through a linker. Antibodies or antigen binding fragments thereof of the disclosure may be conjugated (covalently or non-covalently linked) to a toxin or they may be linked to a toxin through a linker. Suitable linkers are known in the art, and would be apparent to the skilled person. The toxin may be any toxin that can elicit a therapeutic effect. The toxin may be an enzymatically active toxin of bacterial, fungal, plant or animal origin or a synthetic toxin, or fragments thereof.

The use of antibody-drug conjugates (ADCs), e.g., immunoconjugates, for the local delivery of cytotoxic or cytostatic agents to kill or inhibit tumor cells in the treatment of cancer (Syrigos and Epenetos (1999) Anticancer Research 19:605-614; Niculescu-Duvaz and Springer (1997) Adv. Drg Del. Rev. 26:151-172; U.S. Pat. No. 4,975,278) theoretically allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., Lancet pp., 1986:603-05; Thorpe, (1985) “Antibody Carriers Of Cytotoxic Agents In Cancer Therapy: A Review,” in Monoclonal Antibodies '84: Biological And Clinical Applications, A. Pinchera et al. (eds.), pp. 475-506). Efforts to design and refine ADC have focused on the selectivity of monoclonal antibodies (mAbs) as well as drug-linking and drug-releasing properties. Both polyclonal antibodies and monoclonal antibodies have been reported as useful in these strategies (Rowland et al., (1986) Cancer Immunol. Immunother., 21:183-87). Drugs used in these methods include daunomycin, doxorubicin, methotrexate, and vindesine (Rowland et al., (1986) supra).

Toxins useful as therapeutics are known to those skilled in the art. Toxins used in antibody-toxin conjugates include bacterial toxins such as diphtheria toxin, plant toxins such as ricin, small molecule toxins such as geldanamycin (Mandler et al (2000) Jour. of the Nat. Cancer Inst. 92 (19): 1573-1581; Mandler et al (2000) Bioorganic & Med. Chem. Letters 10:1025-1028; Mandler et al (2002) Bioconjugate Chem. 13:786-791), maytansinoids (US20050169933 A1; EP 1391213; Liu et al., (1996) Proc. Natl. Acad. Sci. USA 93:8618-8623), and calicheamicin (Lode et al (1998) Cancer Res. 58:2928; Hinman et al (1993) Cancer Res. 53:3336-3342). Other toxins include plant and bacterial toxins, such as, abrin, alpha toxin, exotoxin, gelonin, pokeweed antiviral protein, and saporin. Toxins can effect their cytotoxic and cytostatic effects by mechanisms including tubulin binding, DNA binding, or topoisomerase inhibition.

In some embodiments, the agent that selectively binds to CD33 comprises an antibody that selectively binds CD33, or an antigen binding fragment thereof, conjugated to a toxin.

In some embodiments, the agent that selectively binds to CD33 selectively binds to amino acids encoded by exon 2 of CD33. In some embodiments, the subject is treated with a chemotherapeutic agent within thirty days of the administration of the agent that selectively binds to CD33. In some embodiments, the chemotherapeutic agent comprises cytarabine (Ara-C), daunorubicin hydrochloride, and/or etoposide phosphate, or any other chemotherapeutic agent as described elsewhere herein.

In some embodiments, the subject is administered cytarabine at a low dose when the summation of the assigned genotype scores (e.g., the ACS10 score), calculated as described herein, is greater than zero (0) (e.g., 1, 2, 3, 4, 5, 6, 7). A “low” dose of cytarabine (LDAraC, or LDAC) is an art-recognized dosage which generally comprises less than 1 g/m², administered twice daily (e.g., every twelve (12) hours) (see, for example, Wu, et al., (2017), supra; Powell, et al. (1989), Low-dose ara-C therapy for acute myelogenous leukemia in elderly patients. Leukemia 3 (1): 23-28). In some embodiments, a low dose of cytarabine comprises about 50 mg/m², about 60 mg/m², about 70 mg/m², about 80 mg/m², about 90 mg/m², about 100 mg/m², about 120 mg/m², about 140 mg/m², about 160 mg/m², about 180 mg/m², about 200 mg/m², about 250 mg/m², about 300 mg/m², about 350 mg/m², about 400 mg/m², about 500 mg/m², about 600 mg/m², about 700 mg/m², about 800 mg/m², about 900 mg/m², about 1 g/m², about 1.1 g/m², about 1.2 g/m², about 1.3 g/m², about 1.4 g/m², or about 1.5 g/m², administered twice daily (e.g., every twelve (12) hours). In some embodiments, a low dose of cytarabine comprises a dose in a range of about 50 mg/m² to about 80 mg/m², about 60 mg/m² to about 90 mg/m², about 70 mg/m² to about 100 mg/m², about 80 mg/m² to about 110 mg/m², about 90 mg/m² to about 120 mg/m², about 100 mg/m² to about 130 mg/m², about 150 mg/m² to about 200 mg/m², about 200 mg/m² to about 300 mg/m², about 300 mg/m² to about 500 mg/m², about 400 mg/m² to about 800 mg/m², about 500 mg/m² to about 1 g/m², about 800 mg/m² to about 1.2 g/m², or about 1 g/m² to about 1.5 g/m², administered twice daily (e.g., every twelve (12) hours). In some embodiments, a low dose of cytarabine is 100 mg/m², administered twice daily (e.g., every twelve (12) hours).

As will be understood, a LDAC may also comprise about 10 mg/m² for patients over a certain age, for example patients over the age of 55. In some embodiments, wherein the subject is over the age of 55, a low dose of cytarabine comprises about 5 mg/m², about 10 mg/m², about 20 mg/m², about 30 mg/m², about 40 mg/m², about 50 mg/m², about 60 mg/m², about 70 mg/m², about 80 mg/m², about 90 mg/m², about 100 mg/m², about 120 mg/m², about 140 mg/m², about 160 mg/m², about 180 mg/m², about 200 mg/m², about 250 mg/m², about 300 mg/m², about 350 mg/m², about 400 mg/m², about 500 mg/m², about 600 mg/m², about 700 mg/m², about 800 mg/m², about 900 mg/m², about 1 g/m², about 1.1 g/m², about 1.2 g/m², about 1.3 g/m², about 1.4 g/m², or about 1.5 g/m², administered twice daily (e.g., every twelve (12) hours). In some embodiments, wherein the subject is over the age of 55, a low dose of cytarabine comprises a dose in a range of about 5 mg/m² to about 20 mg/m², about 10 mg/m² to about 30 mg/m², about 20 mg/m² to about 50 mg/m², about 30 mg/m² to about 60 mg/m², about 50 mg/m² to about 80 mg/m², about 60 mg/m² to about 90 mg/m², about 70 mg/m² to about 100 mg/m², about 80 mg/m² to about 110 mg/m², about 90 mg/m² to about 120 mg/m², about 100 mg/m² to about 130 mg/m², about 150 mg/m² to about 200 mg/m², about 200 mg/m² to about 300 mg/m², about 300 mg/m² to about 500 mg/m², about 400 mg/m² to about 800 mg/m², about 500 mg/m² to about 1 g/m², about 800 mg/m² to about 1.2 g/m², or about 1 g/m² to about 1.5 g/m², administered twice daily (e.g., every twelve (12) hours). In some embodiments, wherein the subject is over the age of 55, a low dose of cytarabine comprises 10 mg/m², administered twice daily (e.g., every twelve (12) hours).

In some embodiments, the subject is administered cytarabine at an intermediate dose when the summation of the assigned genotype scores (e.g., the ACS10 score), calculated as described herein, is between negative one (−1) and one (1) (e.g., −1, 0, 1). An “intermediate” dose of cytarabine (IDAraC, or IDAC) is an art-recognized dosage which generally comprises between 1 g/m² and 2 g/m², administered twice daily (e.g., every twelve (12) hours) (see, for example, Wu, et al., (2017), supra). In some embodiments, an intermediate dose of cytarabine comprises about 500 mg/m², about 600 mg/m², about 700 mg/m², about 800 mg/m², about 900 mg/m², about 1.0 g/m², about 1.1 g/m², about 1.2 g/m², about 1.3 g/m², about 1.4 g/m², about 1.5 g/m², about 1.6 g/m², about 1.7 g/m², about 1.8 g/m², about 1.9 g/m², about 2.0 g/m², about 2.1 g/m², about 2.2 g/m², about 2.3 g/m², about 2.4 g/m², or about 2.5 g/m². In some embodiments, an intermediate dose of cytarabine comprises a dose in a range of about 500 mg/m² to about 700 mg/m², about 600 mg/m² to about 800 mg/m², about 700 mg/m² to about 900 mg/m², about 800 mg/m² to about 1 g/m², about 900 mg/m² to about 1.1 g/m², about 1.0 g/m² to about 1.2 g/m², about 1.1 g/m² to about 1.3 g/m², about 1.2 g/m² to about 1.4 g/m², about 1.3 g/m² to about 1.5 g/m², about 1.4 g/m² to about 1.6 g/m², about 1.5 g/m² to about 1.7 g/m², about 1.6 g/m² to about 1.8 g/m², about 1.7 g/m² to about 1.9 g/m², about 1.8 g/m² to about 2.0 g/m², about 1.9 g/m² to about 2.1 g/m², about 2.0 g/m² to about 2.2 g/m², about 2.1 g/m² to about 2.3 g/m², about 2.2 g/m² to about 2.4 g/m², or about 2.3 g/m² to about 2.5 g/m². In some embodiments, an intermediate dose of cytarabine is 1.5 g/m², administered twice daily (e.g., every twelve (12) hours).

Aspects of the disclosure relate to methods comprising administering clofarabine to a subject (e.g., a subject characterized as “low ACS10” or having a “low ACS10 score”. Clofarabine is a purine nucleoside antimetabolite used for treating AML. Clofarabine may be administered orally or intravenously (IV). In some embodiments, the dosage of clofarabine administered to a subject ranges from about 5 mg/m², about 10 mg/m², about 20 mg/m², about 30 mg/m², about 40 mg/m², about 50 mg/m², about 60 mg/m², or about 70 mg/m² once per day. In some embodiments, the dosage of clofarabine administered to a subject is between about 50 mg/m² and about 60 mg/m² (e.g., 50 mg/m², 51 mg/m², 52 mg/m², 53 mg/m², 54 mg/m², 55 mg/m², 56 mg/m², 57 mg/m², 58 mg/m², 59 mg/m², or 60 mg/m²). In some embodiments, a subject is administered a dose of clofarabine once per week, for 2, 3, 4, 5, or 6 weeks.

### Computer Systems

Techniques as described herein may yield more accurate diagnosis and treatment recommendations for specific subjects. Such techniques involve collecting and processing data on a sufficient number of genes (e.g., CDA, CMPK1, NME4, SLC29A1, RRM2, DCK, RRM1, CTPS1, and SLC28A3) to produce data sets including adequate information to calculate an ACS10 score using an algorithm described herein. The collection and/or processing of such data may be controlled by execution of a computing device.

The invention is operational with numerous other general purpose or special purpose computing system environments or configurations. Examples of well-known computing systems, environments, and/or configurations that may be suitable for use with the invention include, but are not limited to, personal computers, server computers, smartphones, tablets, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The computing environment may execute computer-executable instructions, such as program modules. Generally, program modules include routines, programs, objects, components, data structures, etc. that perform particular tasks or implement particular abstract data types. Some embodiments may also be practiced in distributed computing environments where tasks are performed by remote processing devices that are linked through a communications network. These distributed systems may be what are known as enterprise computing systems or, in some embodiments, may be “cloud” computing systems. In a distributed computing environment, program modules may be located in both local and/or remote computer storage media including memory storage devices.

In some embodiments, a system comprises a detection apparatus. In some embodiments, a detection apparatus is a microplate reader (e.g., fluorescence microplate reader, UV microplate reader, photometer microplate reader, etc.), or a sequencing machine (e.g., a nanopore sequencing machine, a next-generation sequencing machine, an RNA-seq machine, etc.). In some embodiments, the detection apparatus is electronically connected to a computer (e.g., a computer containing a set of executable instructions for performing methods described by the disclosure).

### EXAMPLES

### Example 1

For the last five decades, cytarabine, also known as ara-C, has been the mainstay of acute myeloid leukemia (AML) chemotherapy primarily given in combination with anthracyclines. However, standard ara-C containing chemotherapy fails to induce remission in roughly 10-15% of children. Among those who achieve remission, approximately 40% relapse. This inter-patient variation in treatment response, development of resistance, and high risk of relapse remain major hurdles to effective AML chemotherapy.

Ara-C is a pro-drug requiring activation to ara-CTP by multiple phosphorylation steps. Incorporation of ara-CTP in place of dCTP results in chain termination, thereby blocking DNA and RNA synthesis and causing leukemic cell death. Thus, intracellular abundance of ara-CTP formation is one of the significant determinants of treatment response. Previously, multiple genes have been sequenced in cytarabine metabolic pathway and reported SNPs of functional and clinical relevance. Despite these efforts, a comprehensive evaluation of genetic variation in the key ara-C pathway genes for association with clinical outcome in AML is largely lacking.

The present findings are presented from a comprehensive evaluation of 94 SNPs within 16 ara-C metabolic pathway genes for association with multiple clinical outcome endpoints in pediatric AML patients. A multi-step approach was used to develop a composite pharmacogenomics-based polygenetic SNP Score composed of the 10 most informative SNPs as related to ara-C (designated as “ACS10”). Patients with low-score (ACS10 score>0) had worse outcome as compared to patients within high-score group (ACS10 score>0) in a cohort of clinical trial (AML02) patients. The ACS10 score was further validated in an independent pediatric AML cohort treated under the Children's Oncology Group (“COG”) AAML0531 standard arm. The results hold promise in not just providing a pharmacogenomics-based biomarker to identify patients with high risk of unfavorable response, but also in helping to provide potential alternate strategies in these patients.

### Materials and Methods

### Discovery Cohort

### AML02 Multicenter Clinical Trial (ClinicalTrials.Gov Identifier: NCT00136084):

One hundred and sixty-six pediatric AML patients treated under the multicenter AML02 clinical trial protocol were included in the study. Details of study design and clinical outcome have been described elsewhere. Briefly, patients with de novo AML were randomized to receive either high (3 g/m², given every 12 h on days 1, 3, and 5; this condition is designated “HDAC”) or low dose (100 mg/m² given every 12 h on days 1-10; this condition is designated “LDAC”) cytarabine along with daunorubicin (50 mg/m² on days 2, 4 and 6) and etoposide (100 mg/m² on days 2-6) as a first course of chemotherapy with subsequent treatment tailored to response and risk classification. For initial risk assignment, patients with cytogenetic translocations comprisingt [8;21] (e.g., translocation between chromosome 8 and 21), inv (16), ort [9;11] were classified as low-risk. Patients with features such as deletion of chromosome 7 (−7), presence of FLT3-ITD mutation (FLT-ITD), cytogenetic translocation comprisingt [6;9], megakaryoblastic leukemia (AMKL), treatment-related AML, or AML arising from MDS were classified as high-risk AML. Patients lacking any of the low or high-risk group features were provisionally classified as standard risk AML. Patient risk classifications were updated on the basis of minimal residual disease evaluations. Clinical endpoints used in this evaluation were defined as follows: i) Minimal residual disease after induction 1 (“MRD1”) (patients were defined as MRD positive if they had ≥1 leukemic cell per 1000 mononuclear bone marrow cells (≥0.1%); ii) Complete remission defined as trilineage hematopoietic recovery with less than 5% blasts in the marrow; iii) Event-free survival (“EFS”) defined as the time from study enrollment to induction failure, relapse, secondary malignancy, death, or study withdrawal for any reason, with event-free patients censored on the date of last follow-up; and iv) Overall survival (“OS”) defined as the time from study enrollment to death, with living patients censored on the date of last follow-up. St. Jude Institutional Review Board approved the study, and informed consent was obtained from parents/guardians and consents/assents from the individuals.

### Validation Cohort

### COG-AAML0531 (ClinicalTrials.Gov Identifier NCT00372593):

AAML0531 clinical trial enrolled previously untreated AML patients (1 month to 29.9 years old) who were randomized to receive ADE (cytarabine 100 mg/m²/dose twice per day for days alongside with daunorubicin and etoposide-equivalent to LDAC arm of St. Jude AML02) with or without the addition of 1 dose in induction 1 and 1 dose in intensification of CD33-targeting drug gemtuzumab ozogamicin (ADE+GO arm). Genotype data on 10 SNPs that were part of the ACS10 score in the discovery cohort was generated in 931 patients (n=465 in standard ADE arm and n=466 in ADE+GO arm). Since the addition of GO has been shown to positively influence the outcome, for the purpose of ACS10 initial validation, analysis was restricted to patients within the standard ADE arm (this arm is also comparable to the treatment regimen used in the discovery cohort). In subsequent analysis to check whether ACS10 score has impact on the GO addition, the evaluation of patients from the ADE+GO arm was also considered. Table 3 provides summary of patient characteristics for the AML02 and COG cohorts.

### Genotyping

Genomic DNA from patients enrolled in the multi-site St. Jude AML02 trial was genotyped for 155 SNPs in 16 genes of relevance to ara-C pharmacology using sequenom iPlex platform that uses MALDI-TOF based chemistry at University of Minnesota, Biomedical Genomics Center. SNPs were selected based on previously reported studies. For SNPs lacking literature on genetic variation, SNPs were selected to capture LD blocks (European and African ancestry) on a gene. Of 155 SNPs, 1 SNP was excluded due to low call rate (<90%), 47 SNPs were excluded due to minimum allele frequency of less than 5%, 11 SNPs were excluded due to high LD (r^{2>0.9}) with other SNPs; two SNPs were excluded because of deviation from Hardy-Weinberg equilibrium. Overall, 94 SNPs were included for further association analysis with multiple endpoints described above (listed in Table 4). Genotype for the COG-AAML0531 cohort for the selected SNPs was obtained using sequenom platform or TaqMan genotyping assays, and for a few SNPs was extracted from the data available on Illumina 2.5 Omni array. Genotype calls from multiple platforms were confirmed across multiple samples randomly collected from the cohort.

Association of Ara-C Pathway SNPs with Outcome in the AML02 Cohort

SNP genotype groups in three different modes of inheritance (additive, dominant, and recessive) were tested for association with MRD1 using logistic regression models. Odds ratio (OR) and 95% confidence interval (CI) were calculated for each test. SNPs with association P-value<0.05 were considered significant. Cox-proportional hazard models were used to evaluate association of genotype groups with EFS or OS. Hazard ratio (HR) and 95% CI were calculated for each test. p-value<0.05 was considered statistically significant. Given initial risk group assignments are well-established prognostic factors associated with outcome, outcome association analysis of SNPs with and without adjusting for risk group was also performed to identify SNPs that are associated with outcome independent of risk group.

### Development of Multi-SNP Predictor Models for MRD1, EFS, and OS in AML02

SNPs with p<0.15 in risk-adjusted univariate evaluation with clinical endpoints (MRD1, EFS, and OS) were tested for all possible combinations with a maximum of three SNPs per model in multivariable logistic regression models for association with MRD1 and Cox-proportional hazard models for association with EFS and OS. Analysis for up to 3 SNP combinations was restricted due to computational challenges as increasing the maximum number of SNPs per model drastically increases the number of models as 1000 permutations were run for each model. Models were ordered according to their Bayesian Information Criterion (BIC) and weight in favor of each model. One thousand permutation tests were performed for each model to determine statistical significance. The six unique SNPs from the most significant models for EFS and MRD1 were included in the development of the ACS10 score as described in the results section.

### Definition of the 10-SNP Ara-C Pharmacogenetic SNP Score ACS10

SNPs passing the multiSNP predictor model were utilized for development of an ara-C pharmacogenomics score composed of 10 SNPs-termed as ACS10 score and included 3 SNPs that were part of the best model selected for association with MRD1 or 3 SNPs that were part of the best model selected for association with EFS and four recently reported SNPs (DCK-rs4643786, RRM1-rs11030918, CTPS1-rs12067645, and SLC28A3-rs17343066) that were part of significant models predictive of leukemic cell intracellular levels of ara-CTP (summarized in FIG. 1). The composite ACS10 SNP score is the sum of SNP genotypes that are beneficial minus the sum of SNP genotypes that are detrimental. Overall, ACS10 score was defined by adding the genotype scores which in turn took into account the mode of inheritance (additive, dominant or recessive) and the direction of association of SNPs with outcome (positive for beneficial and negative for detrimental association). Scores were further compressed to classify patients into two groups: low-ACS10 score (score>0) and high-ACS10 score group (scores>0).

### Utility of ACS10 in AML02 and AAML0531

The association of MRD1, EFS, and OS was evaluated with ACS10 scores in the AML02 discovery cohort and the AAML0531 validation cohort. The Kaplan-Meier method was used to estimate the EFS and OS probabilities for well-defined groups of patients. Cox regression models were used to associate ACS10 with EFS and OS and used logistic regression models to associate ACS10 with MRD1. The Wilcoxon rank-sum test and Kruskal-Wallis test were used to compare medians of numeric variables across groups. Chi-square tests and Fisher's exact test were used to evaluate the association among pairs of categorical variables. All p-values are two-sided. All statistical analyses were performed using R software (www.r-project.org).

### Results

The overall study schema is shown in FIG. 1, with details described below.

Ara-C SNPs for Associated with Outcome in AML02

The association of SNPs with MRD1, EFS, and OS was evaluated by performing an unadjusted and initial risk-group adjusted analysis and identified 34 SNPs with at least one significant association with clinical outcome (p<0.05; Table 2). SNP rs10916819 in the 5′UTR of the ara-C inactivating enzyme CDA was associated with an increased rate of MRD1 positivity (OR=1.86, p=0.002), inferior EFS (HR=1.37, p=0.026), and inferior OS (HR=1.61, p=0.009) in risk-adjusted analyses and was significant with all three endpoints in unadjusted analyses as well (all p<0.05, Table 2). A missense coding SNP in CDA (rs2072671) was associated with reduced MRD1 positivity (OR=0.49, p=0.014). Also, a synonymous coding SNP in CDA (rs1048977) was associated with greater MRD1 positivity (OR-4.36, p=0.01). Two CDA intronic SNPs, rs818196 was associated with worse EFS (unadjusted p=0.048), and OS (unadjusted p=0.007, risk-adjusted p=0.019) and rs580032 associated with better EFS (p<0.05). Two SLC28A1 intronic SNPs were associated with worse OS (rs3743162: HR=1.73, p=0.03 and rs4980345: HR=1.4, p=0.037). Two 5′ region SNPs in SLC29A1 were associated with poor EFS (rs507964: HR=1.88, p=0.00004 and rs2396243: HR=1.72, p=0.008). SNPs in other ara-C metabolic pathway genes with association with outcome included: CMPK (rs1044457 with better OS, rs3088062 with lower EFS and OS and rs17103168 with lower MRD positivity), CTPS1 (rs7533657 associated with lower EFS and OS); NME4 (rs5841 with lower MRD1 positivity), DCTD (were associated with inferior EFS and OS (rs4742, rs6552622 and rs2037067), and NT5C2 (rs11598702 and rs1712517 associated with better survival and rs4917384 associated with lower OS) (Table 2).

The multicenter AML02 clinical trial randomly assigned subjects to receive low dose ara-C (LDAC: 100 mg/m²; n=91 patients) or high dose ara-C(HDAC: 3 g/m²; n=75 patients) in combination with daunorubicin and etoposide as the first course of chemotherapy as described above in patient cohort section. Thus, the association of SNPs with outcome within each arm separately was also explored (Table 5). Within the LDAC arm, 25 SNPs were significantly associated with at least one clinical endpoint and 15 SNPs in the HDAC arm. Two SNPs, rs10916819 in CDA and rs507964 in SLC29A1, showed a consistent and significant impact in both arms.

### Development of Ara-C Pharmacogenetic SNP (ACS10) Score

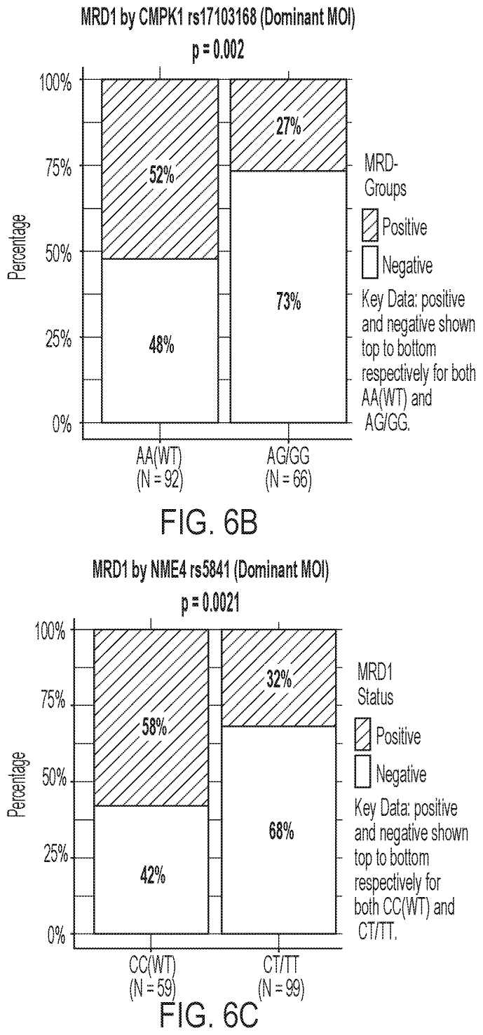

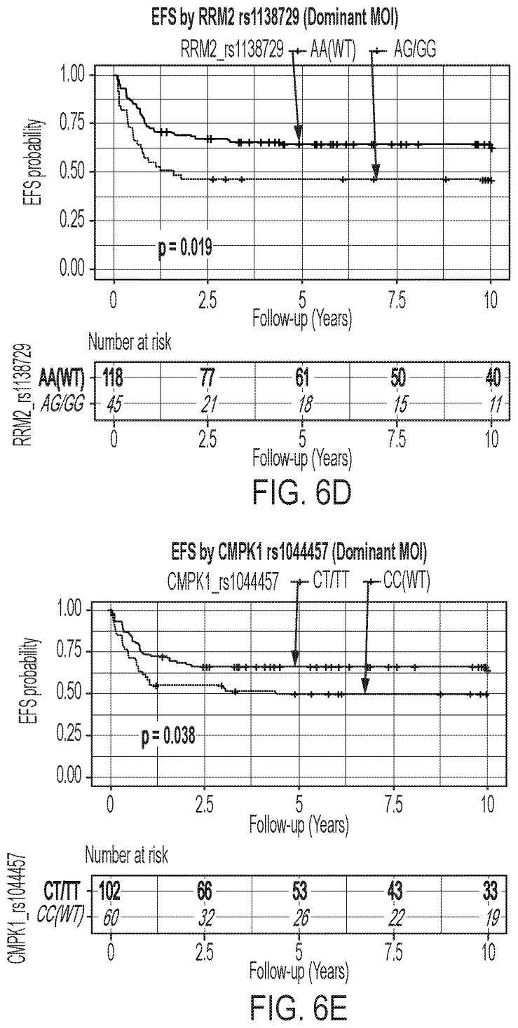

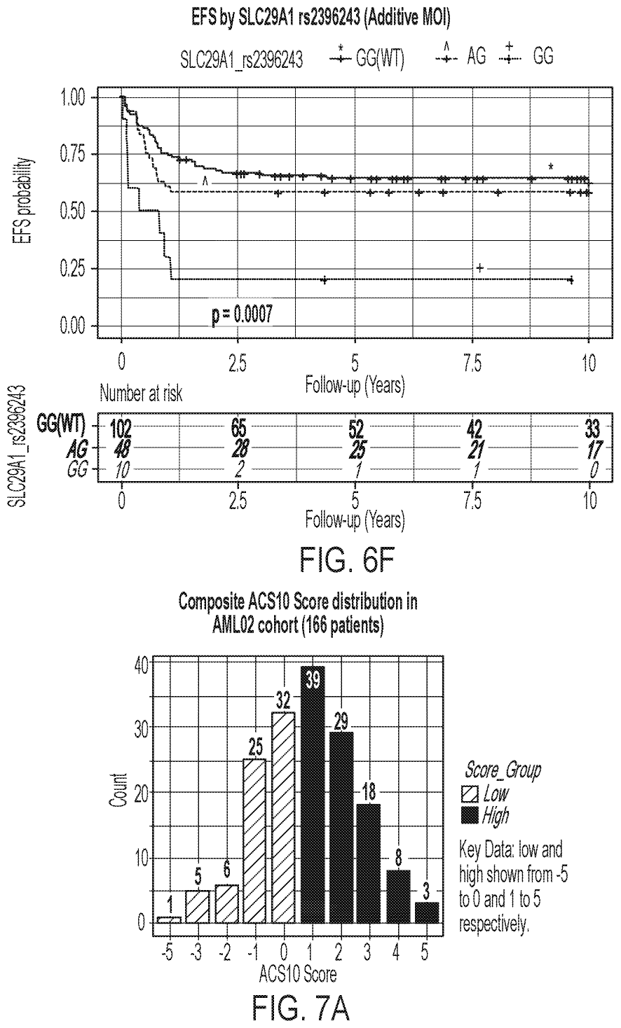

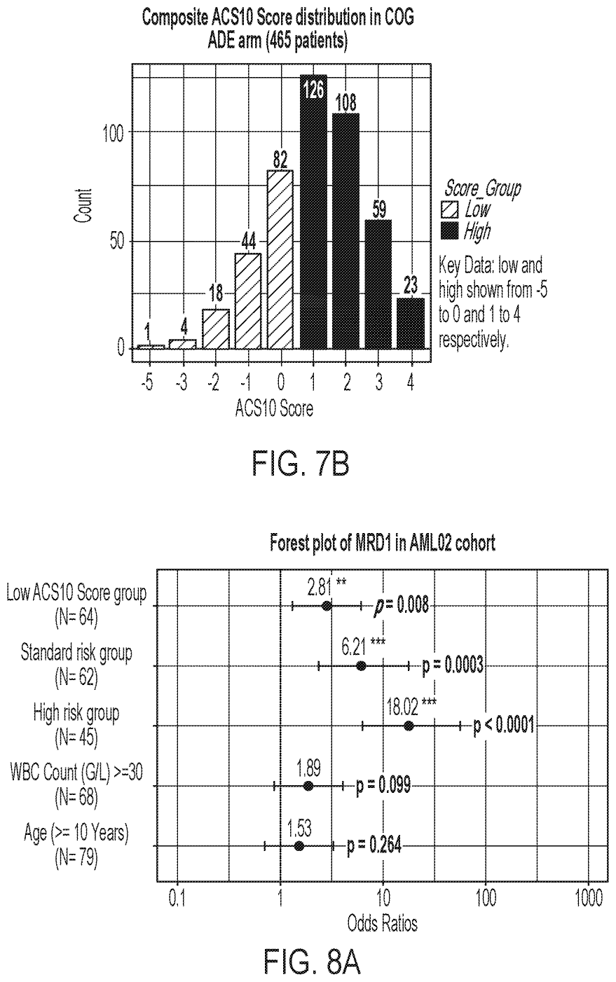

Given multiple SNP combinations can co-occur in a patient and impact the outcome, BIC and permutation modeling approach were used to evaluate all possible SNP-SNP combination models with up to 3 SNPs with MRD1, EFS, and OS as outcome endpoints. The top models selected (lowest BIC and p value) for MRD1 included rs10916819 in CDA, rs17103168 in CMPK1, and rs5841 in NME4 (Tables 6A-6B). For EFS the chosen model included rs2396243 in SLC29A1, rs1044457 in CMPK1, and rs1138729 in RRM2 (Tables 7A-7B). The same SNPs as in the EFS model were also identified in the top OS model. FIGS. 6A-6F show these 6 individual SNPs and association the respective clinical endpoints. A composite ACS10 score was defined with the 6 unique SNPs from the chosen models for MRD1, EFS as described above and previously reported 4 SNPs associated with intracellular ara-CTP levels using similar BIC and permutation approach. Thus, the 10 most informative SNPs were selected for generation of the ACS10 score as described in the methods section and summarized in FIG. 1 and Table 8. Distribution of scores in the patient cohorts is shown in FIGS. 7A-7B. ACS10 ranged from −5 to +5 in AML02 trial and from −5 to +4 in AAML0531 trial.

### ACS10 Predicts Outcome in the AML02 Discovery Cohort

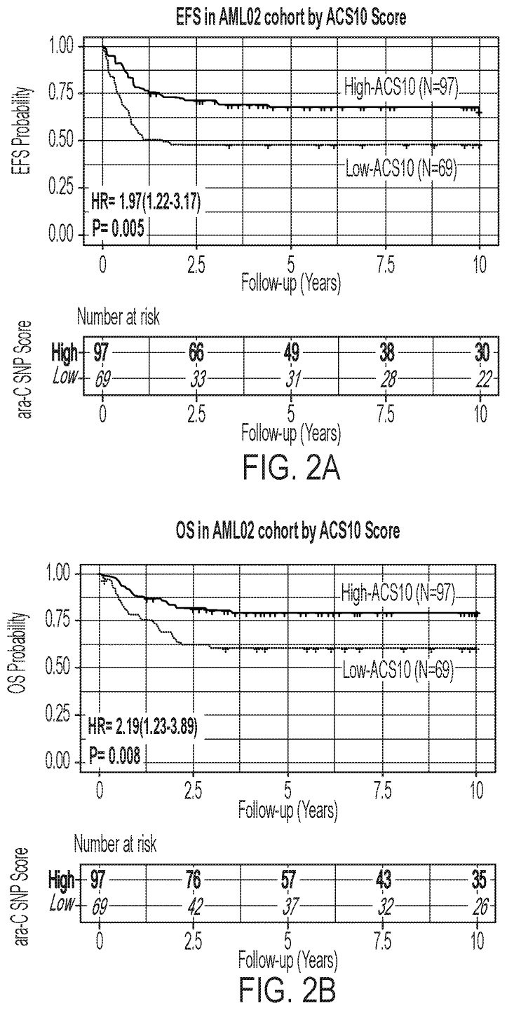

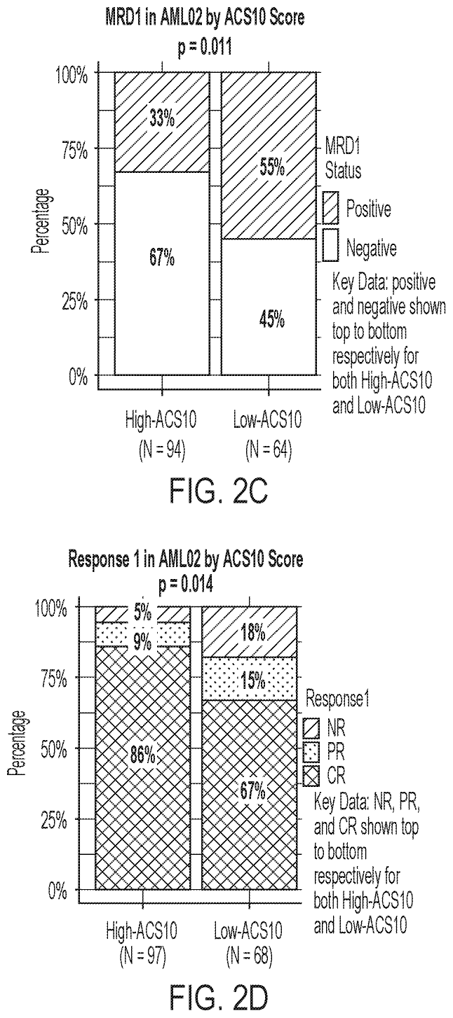

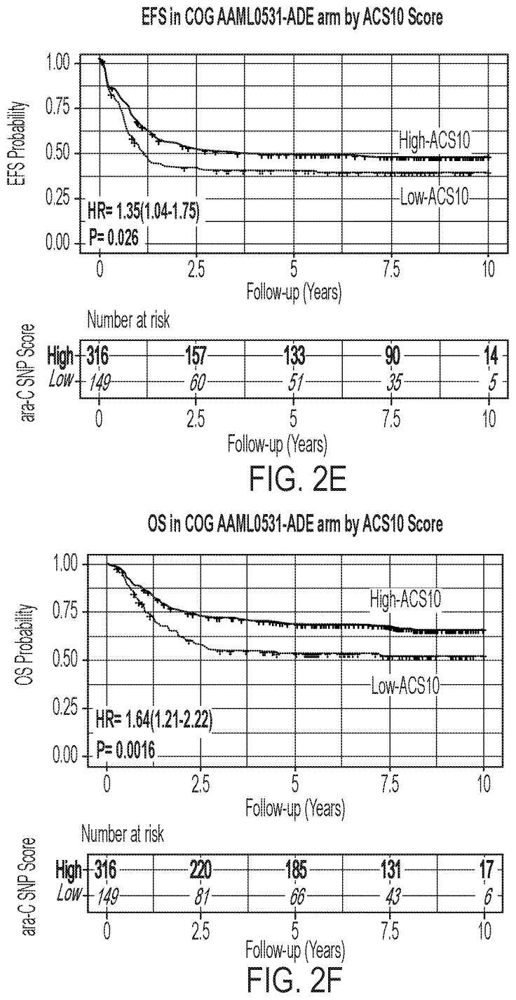

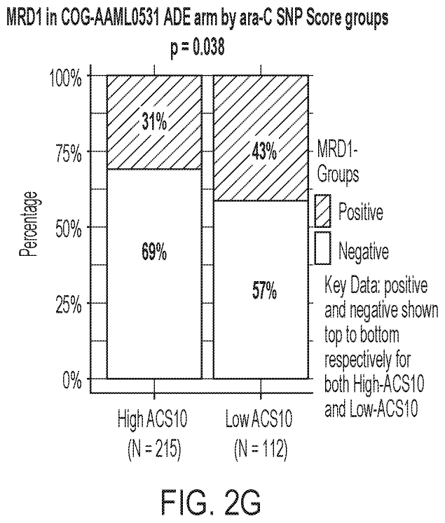

In the AML02 cohort as a whole, each one-point increase in ACS10 associated with better EFS (HR=0.82; 95% CI: 0.71-0.94; p=0.007) and OS (HR=0.81; 95% CI: 0.69-0.96; p=0.012) in an arm-stratified risk-adjusted Cox regression model. Considering the variability in ACS10 score, this association is clinically relevant. A five-point increase in ACS10 score associates with EFS with HR=0.825=0.37 and OS with HR=0.815-0.35. The association also exists with ACS10 score being dichotomized as low-ACS10 (score≤0) or high-ACS10 (score>0). Patients with low-ACS10 score had significantly worse EFS (HR=1.97, 95% CI=1.22-3.17, P=0.005; FIG. 2A), worse OS (HR=2.19, 95% CI=1.23-3.89, P=0.008; FIG. 2B), greater MRD1 positivity (55% Vs. 33%; P=0.011; FIG. 2C) and lower proportion of patients with complete remission after induction I (67% Vs. 86%; P=0.014; FIG. 2D) as compared to patients within high-ACS10 score group. Patient characteristics and outcome within low-ACS10 and high-ACS10 groups within AML02 cohort is shown in Table 9. Race was the only demographic factor found to be significantly different with 73% of black patients as compared to 33% white patients within low-ACS10 score group.

### Validation of ACS10 Score in the COG AAML0531-Cohort (Standard-ADE Chemotherapy Arm)

Given the standard arm of COG-AAML0531 cohort received ara-C, daunorubicin and etoposide based regimen similar to the AML02-discovery cohort thus to avoid the impact of GO addition (GO+ADE arm), the validation of ACS10 score was performed within COG-AAML0531-ADE arm. Consistent with the discovery cohort, in the AAML0531-ADE arm, each one-point increase in ACS10 score was associated with better EFS (HR=0.89; 95% CI: 0.82, 0.97; p=0.007) and OS (HR=0.85; 95% CI: 077, 0.94; p=0.002) in a risk-adjusted Cox model. A five-point increase in ACS10 score is characterized by HR=0.895=0.57 for EFS and HR=0.855=0.45 for OS. The association was also significant with ACS10 dichotomized into high (>0) and low (≤0) groups. Consistent with the discovery cohort, within ADE arm of AAML0531 (n=465), low-ACS10 score group had reduced EFS (HR=1.35, 95% CI=1.04-1.75, p=0.026; FIG. 2E), reduced OS (HR=1.64, 95% CI=1.21-2.22, P=0.0016; FIG. 2F) and greater proportion of MRD positive patients after induction 1 (43% vs. 31% P=0.038; FIG. 2G) as compared to high-ACS10 score group. Distribution of patients characteristics across ACS10 score groups as well outcome results are shown in Table 10 and consistent with AML02 discovery cohort race demonstrated significant difference by score groups with 67% black patients as compared to 29% white patients within low-ACS10 score group.

### ACS10 Score as an Independent Predictor of Treatment Outcomes in AML02 and COG-AAML0531_ADE-Arm Cohorts

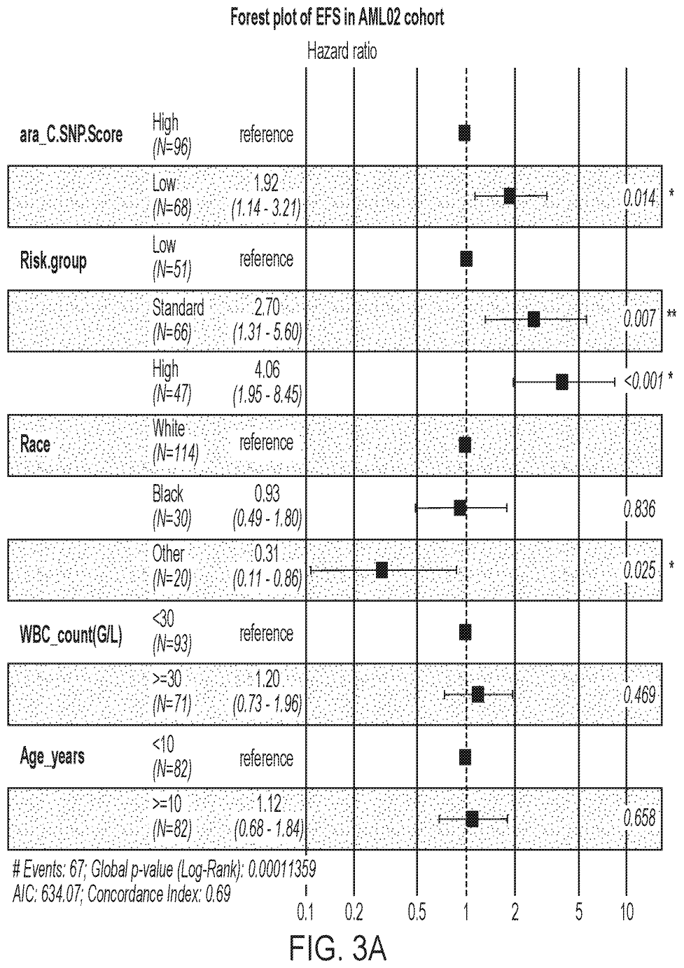

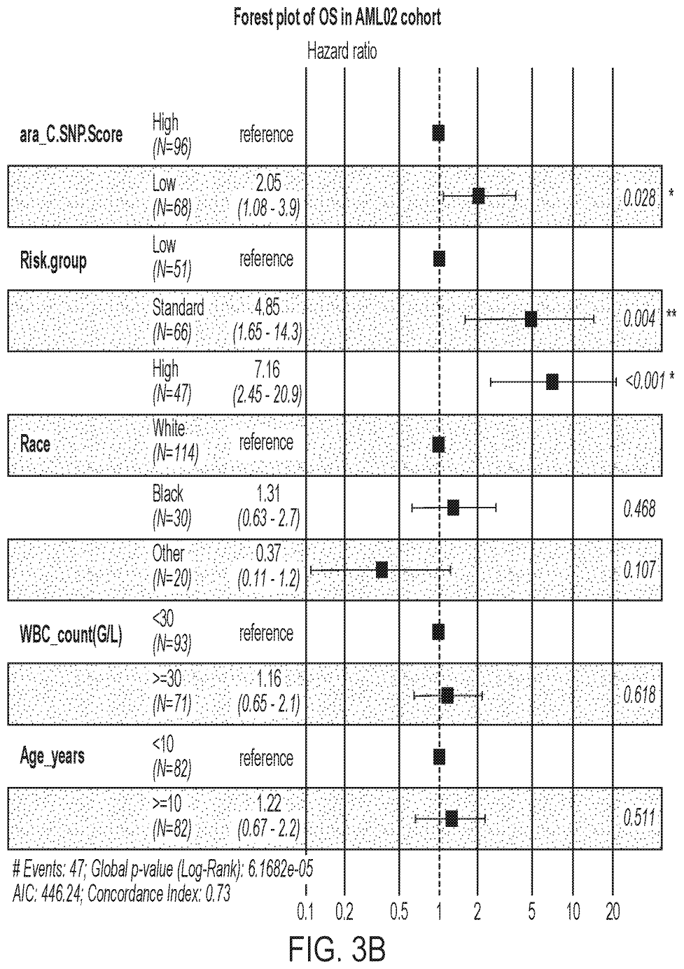

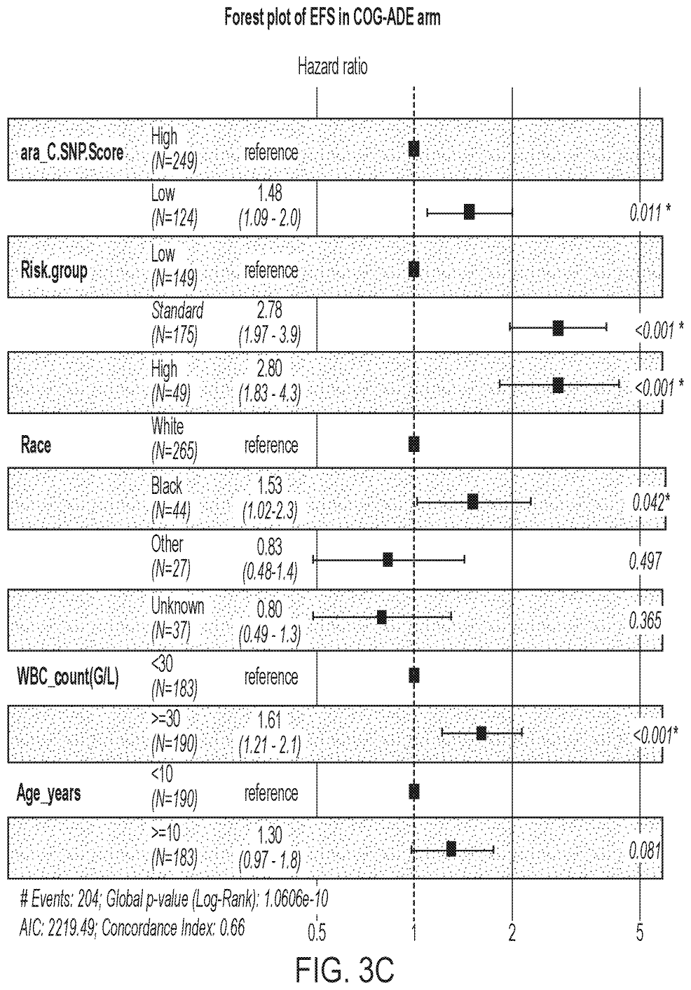

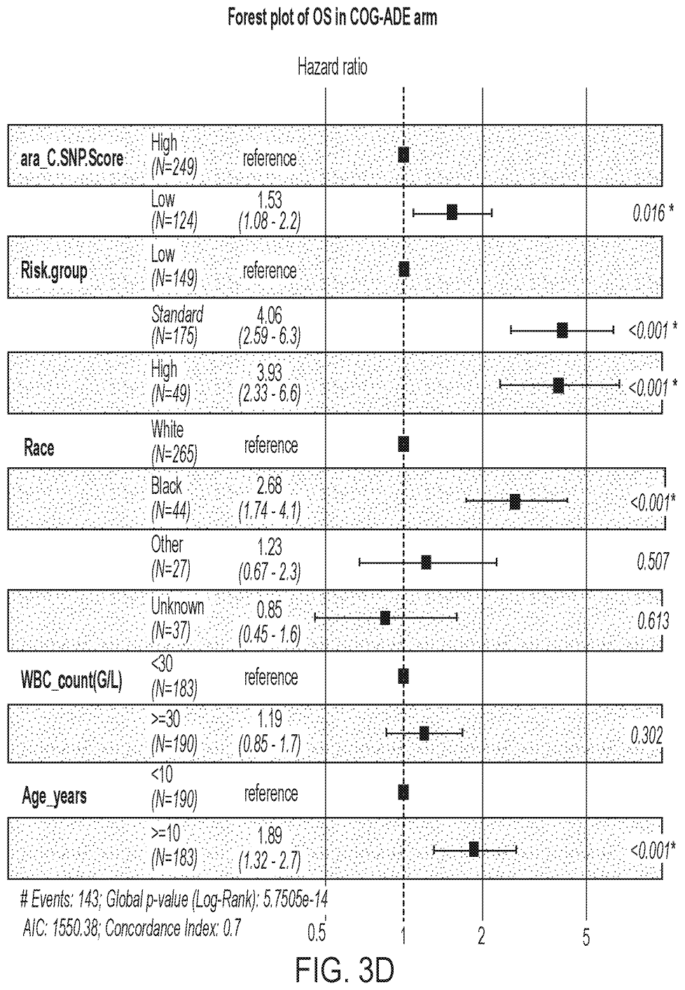

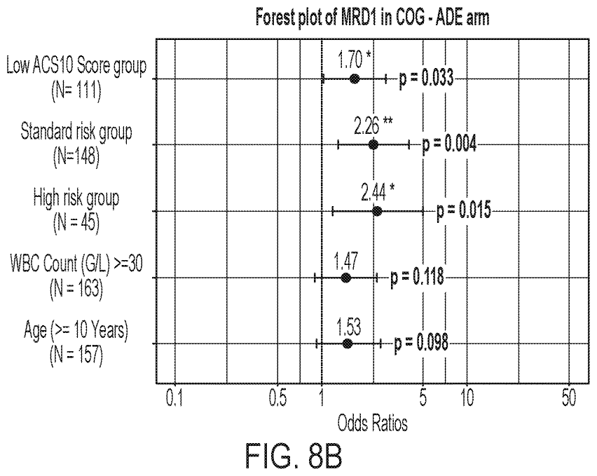

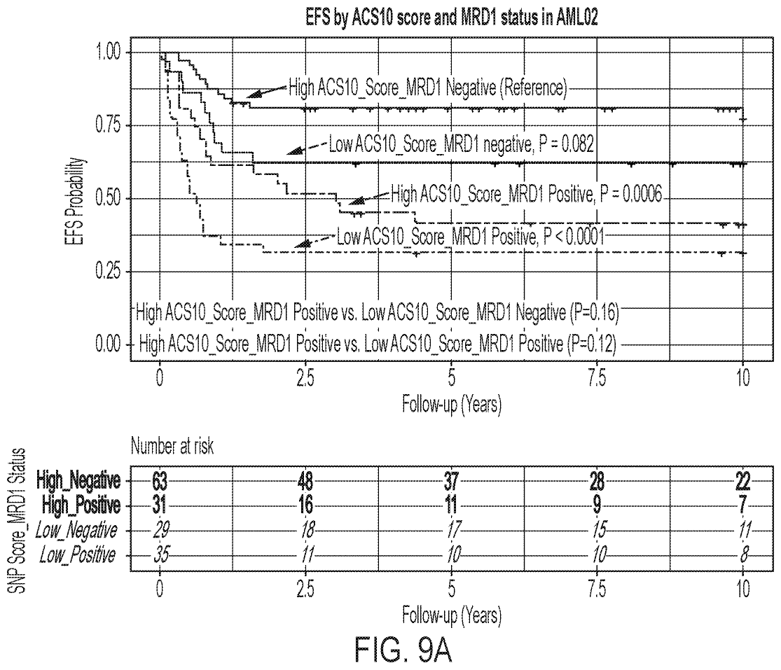

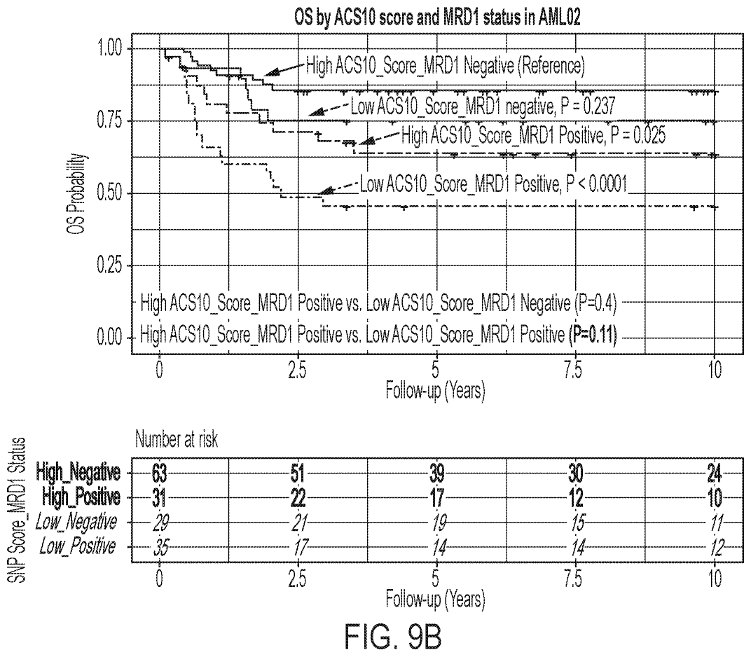

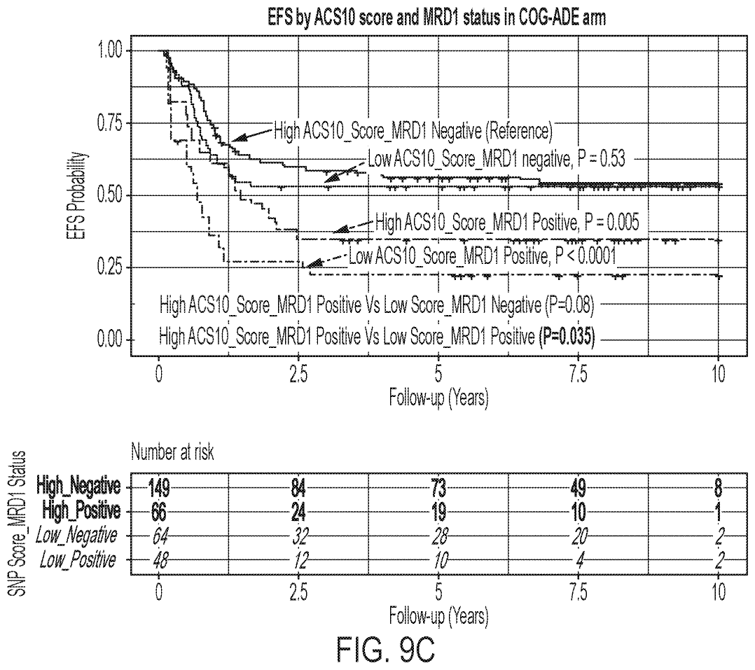

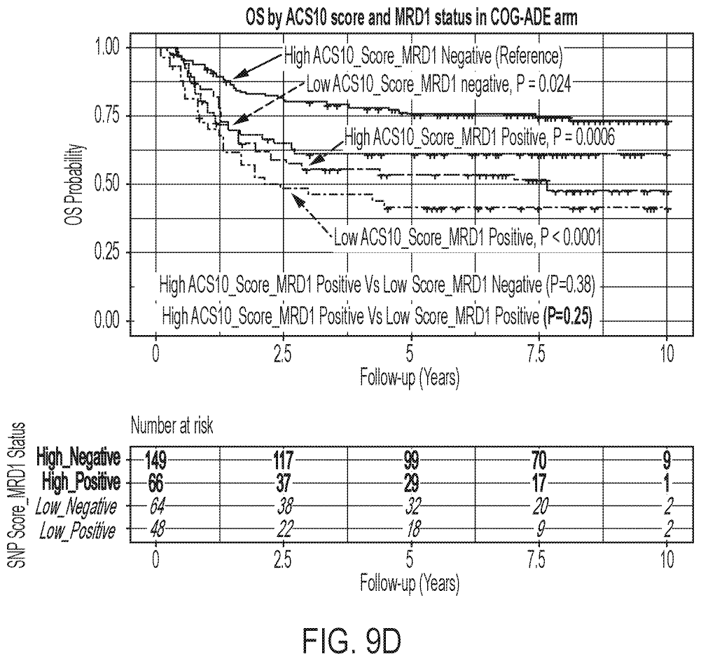

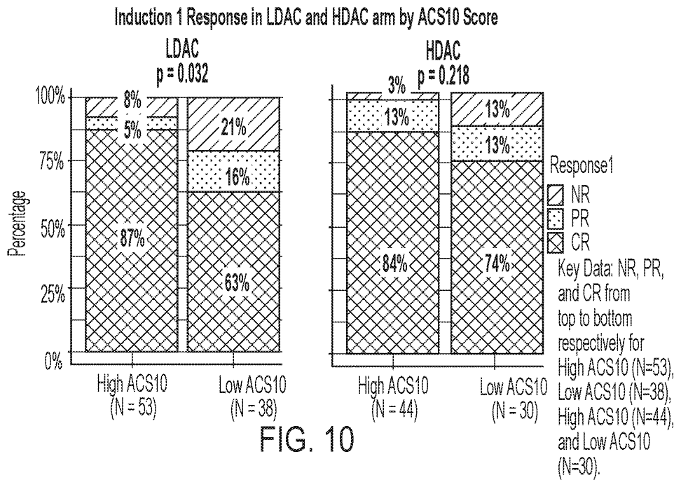

In multivariable Cox proportional hazard models that included ACS10 score groups, initial risk group assignment, race, WBC at diagnosis and age, low-ACS10 score remained a significant and independent predictor of inferior EFS (HR=1.92, 95% CI=1.14-3.21, P=0.014; FIG. 3A) and OS (HR=2.05, 95% CI=1.08-3.9, P=0.028; FIG. 3B) in AML02 cohort. These results were validated in the COG-AAML0531 ADE arm for EFS (HR=1.48, 95% CI=1.09-2, P=0.011; FIG. 3C) and OS (HR=1.53, 95% CI=1.08-2.2, P=0.016; FIG. 3D). Similar results were observed for MRD1 (AML02 cohort: OR=2.81. 95% CI=1.33-6.14, p=0.008; COG-AAML0531-ADE arm cohort, OR=1.70, 95% CI=1.04-2.77, p=0.033; FIGS. 8A-8D). Given that MRD status after induction 1 chemotherapy holds significant prognostic value, ACS10 score groups were evaluated by MRD1 status. Overall patients within ‘MRD1-negative&low-ACS10’ group had better EFS and OS. Using this group as reference, intermediate response was observed in patients within ‘MRD1-negative&low-ACS10’ OR ‘MRD1-positive&high-ACS10’ groups and worst outcome was observed in patients within ‘MRD-positive&high-ACS10’ group in AML02 (FIGS. 9A-9B) and AAML0531-ADE arm cohorts (FIGS. 9C-9D).

### ACS10 Score Prediction is Impacted by Ara-C Dose in LDAC and HDAC Treatment Arms of AML02

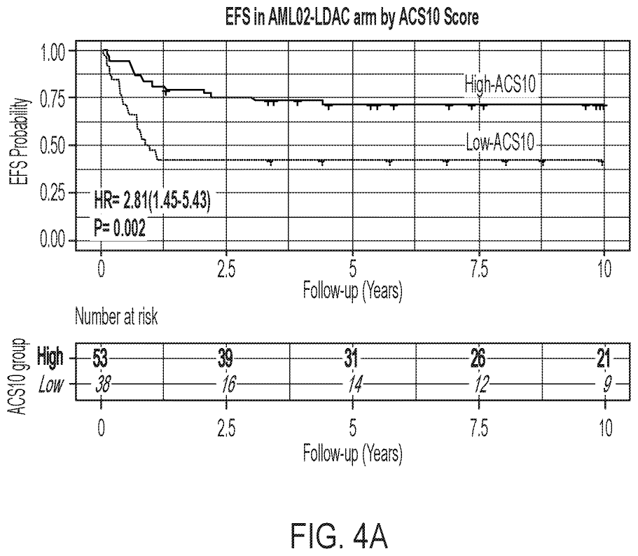

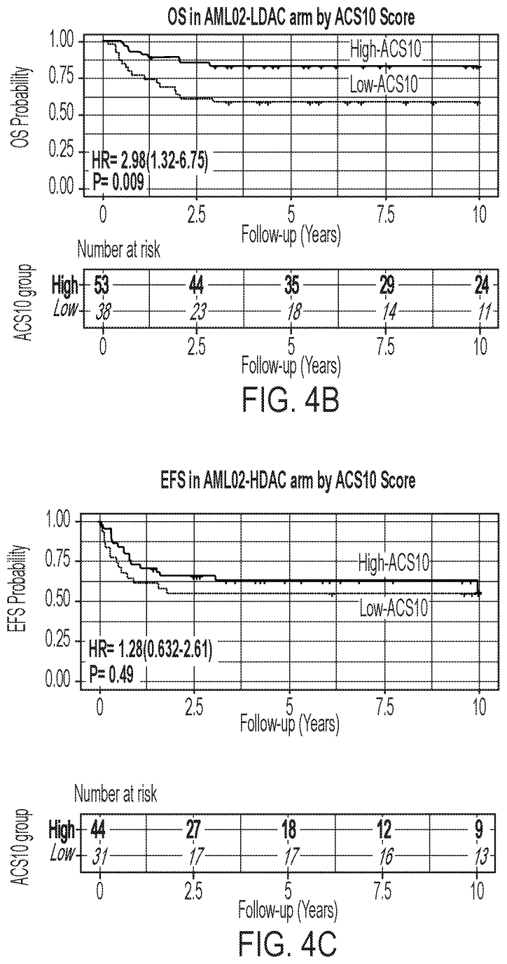

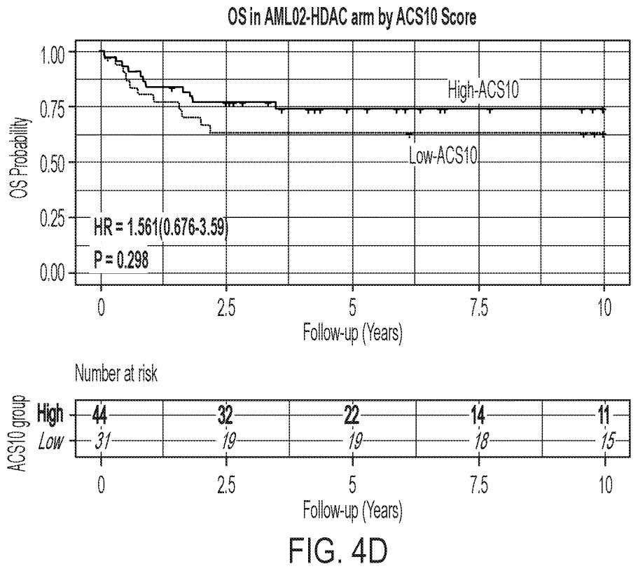

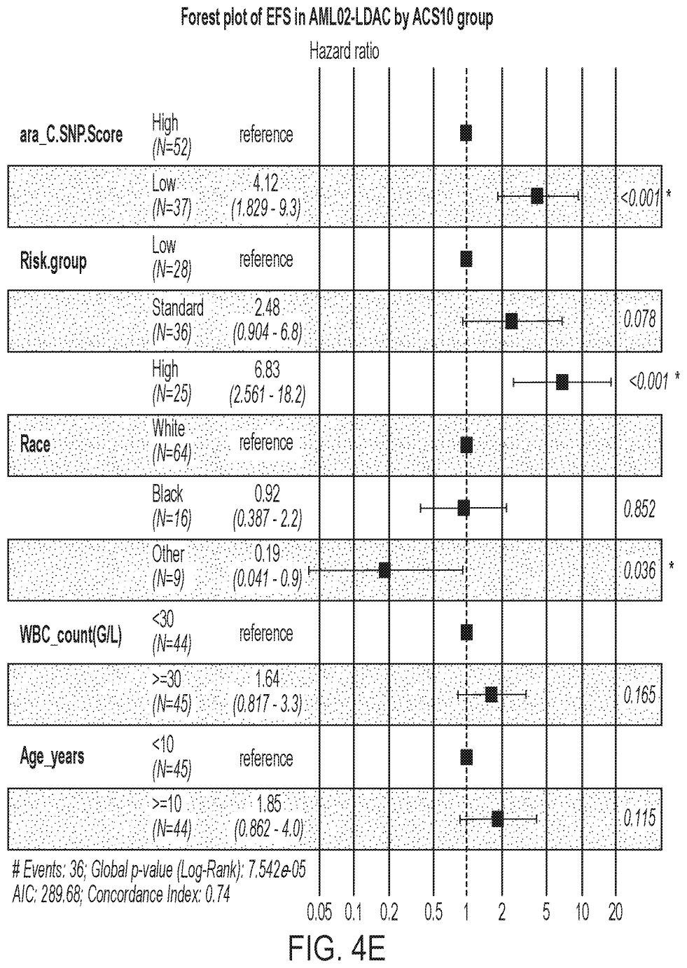

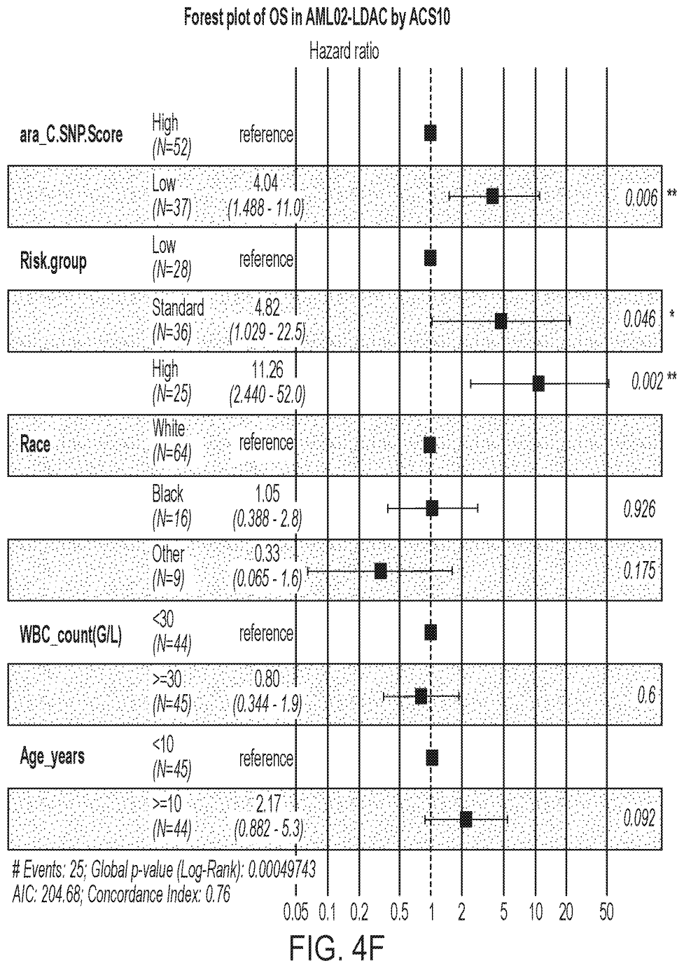

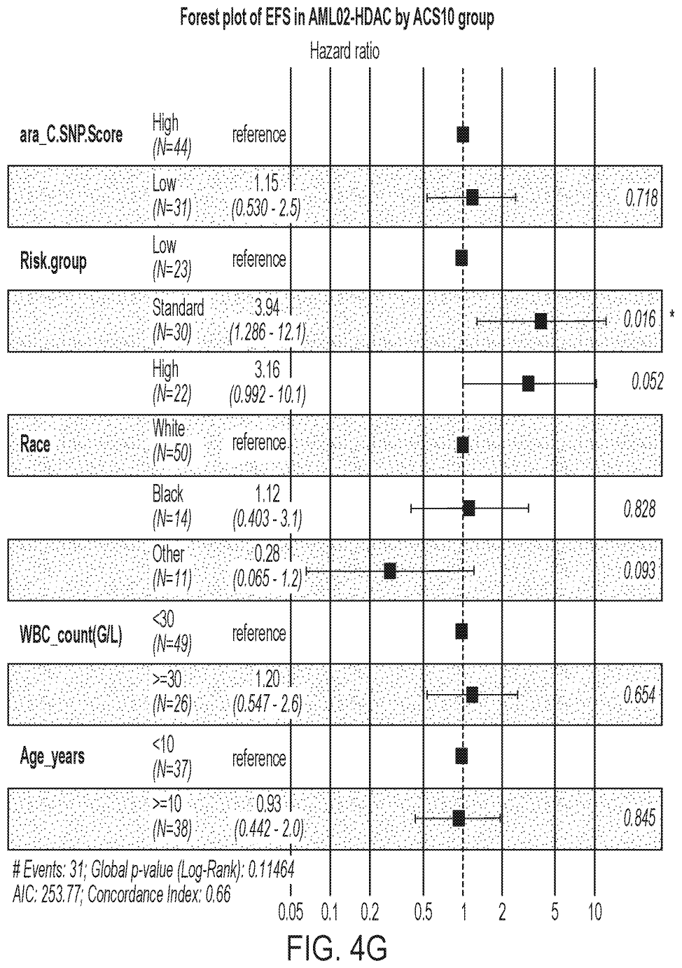

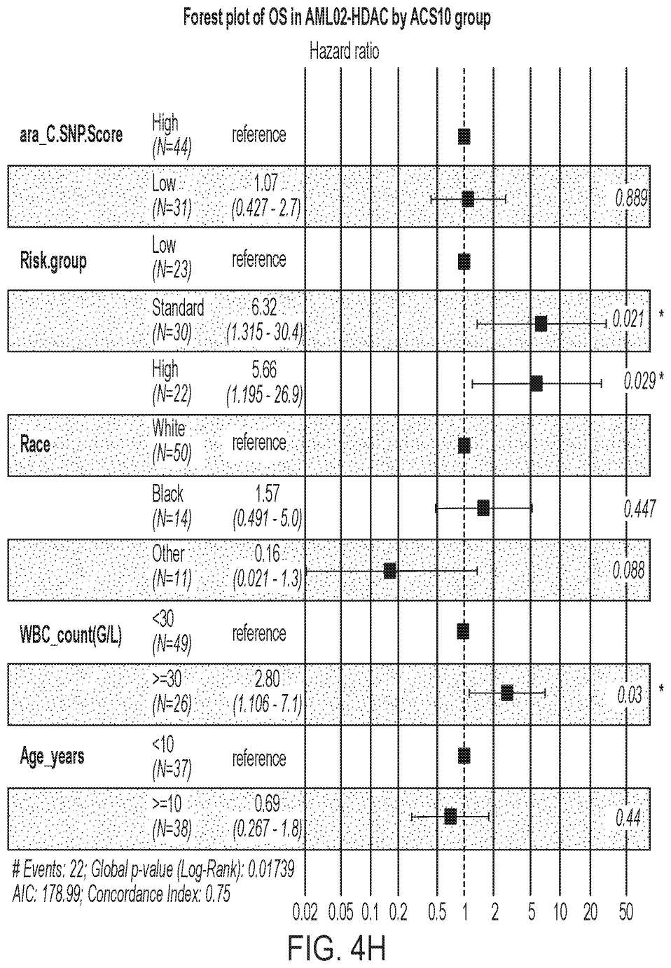

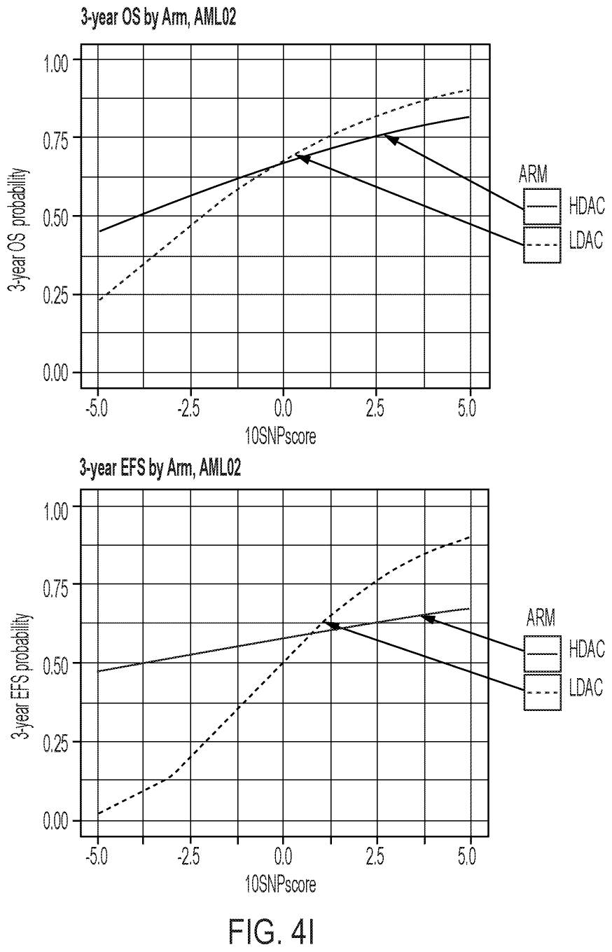

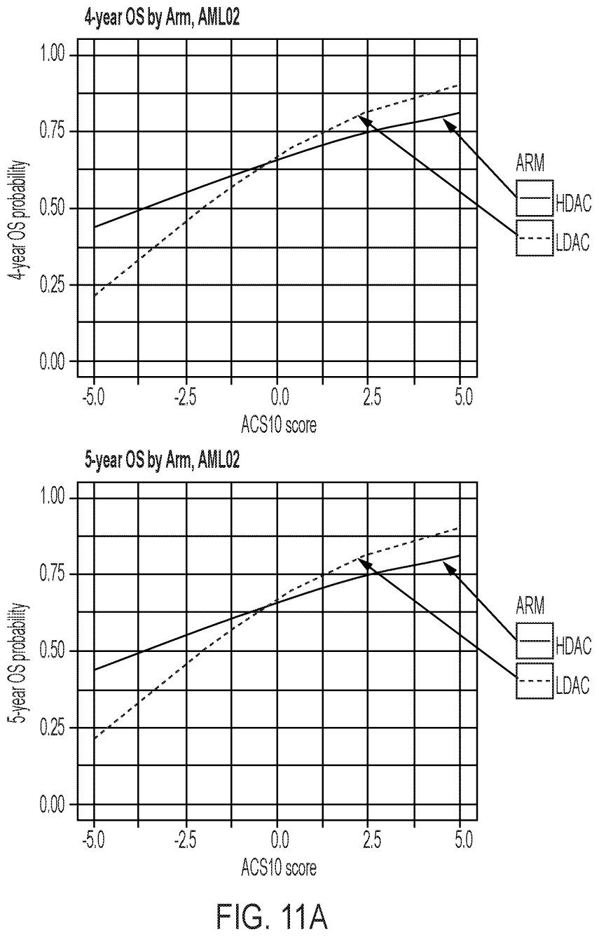

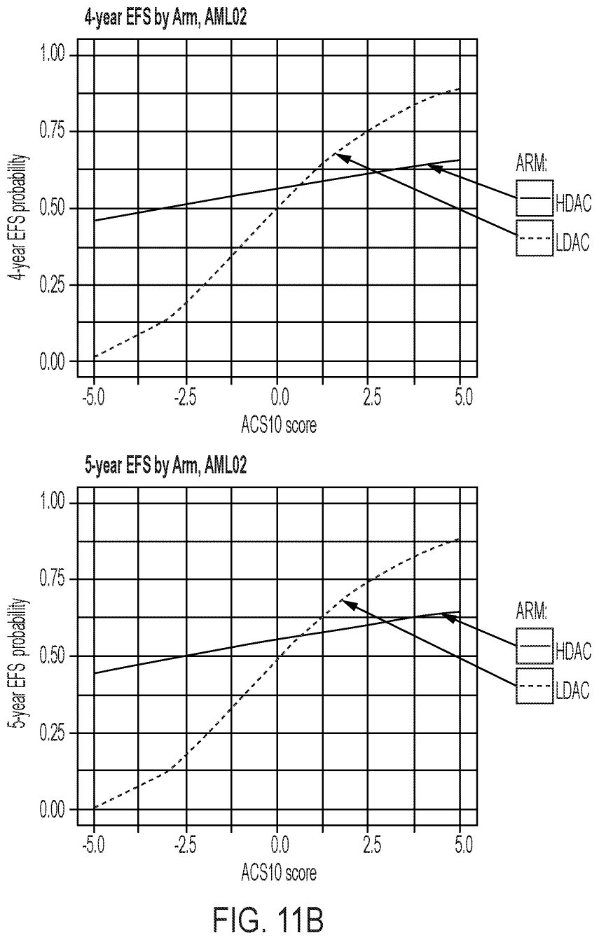

Given patients in AML02 cohort were randomized to receive low or augmented high dose of ara-C, this unique opportunity was leveraged to evaluate performance of ACS10 score within the different ara-C dose treatment arms (of note LDAC and HDAC arms had similar distribution of patients across, disease characteristics, risk group, cytogenetics, race, gender etc. Table 3). Though limited by sample size, results revealed that the impact of ACS10 score differed by ara-C treatment dose. For patients treated in LDAC arm having low-ACS10 score was significant predictor of poor survival as compared to having high-ACS10 score (EFS: HR=2.81, 95% CI=1.45-5.43, P=0.002, FIG. 4A; OS: HR=2.98, 95% CI=1.32-6.75, P=0.009, FIG. 4B) but such an association within HDAC arm was not observed (EFS: HR=1.28, 95% CI=0.632-2.61, P=0.49; FIG. 4C; OS: HR=1.56, 95% CI=0.676-3.59, P=0.298; FIG. 4D). In multivariable Cox proportional hazard models that included ACS10 score groups, initial risk-group assignment, race, WBC at diagnosis and age, low-ACS10 score remained a significant and independent predictor of inferior EFS in LDAC but not HDAC arm (FIGS. 4E-4H). Similar results were obtained for complete remission status after induction 1 (low-ACS10 score vs. high-ACS10 score, LDAC arm, p=0.032 and HDAC arm, p=0.218, FIG. 10). These results suggest that patients within low-ACS10 score group have worse outcome when given low dose ara-C which might be overcome by augmenting the treatment with high dose of ara-C. Thus, the interaction of arm with ACS10 score (as a numeric score) was evaluated in terms of EFS/OS outcomes at fixed-time in a risk-adjusted Cox model. A risk-adjusted Cox model found a statistically significant interaction between arm and score for EFS (p=0.042) but not OS (p=0.42), FIG. 4I. ACS10 was found to be significantly associated with better EFS (HR=0.70 for each one-point increase in ACS10, 95% CI: 0.57-0.87; p=0.001) on the LDAC-arm but not on the augmented HDAC-arm (HR=0.92; 95% CI: 0.75-1.12, p=0.40). The model estimates that patients with ACS10 score of −5 will have a 3-year EFS (OS) of 7.4% (32.8%) with LDAC and 51.2% (50.7%) with HDAC while the patients the ACS10 score of +5 will have a 3-year EFS (OS) of 89.9% (90.7%) with LDAC and 69.0% (82.8%) with HDAC (FIG. 4I). Similar results were obtained for 4-year and 5-year OS and EFS (FIGS. 11A-11B; Table 10).

ACS10 Score by ADE Vs. ADE+GO Treatment Arms in COG-AAML0531

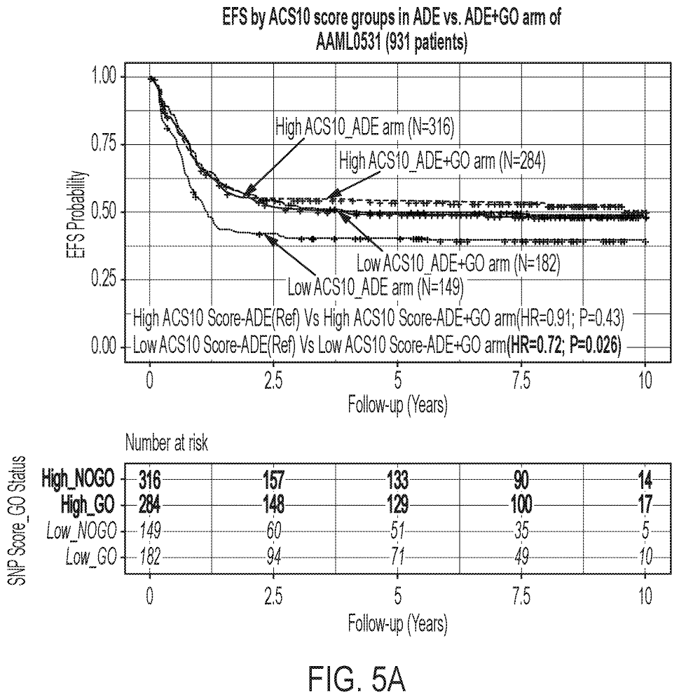

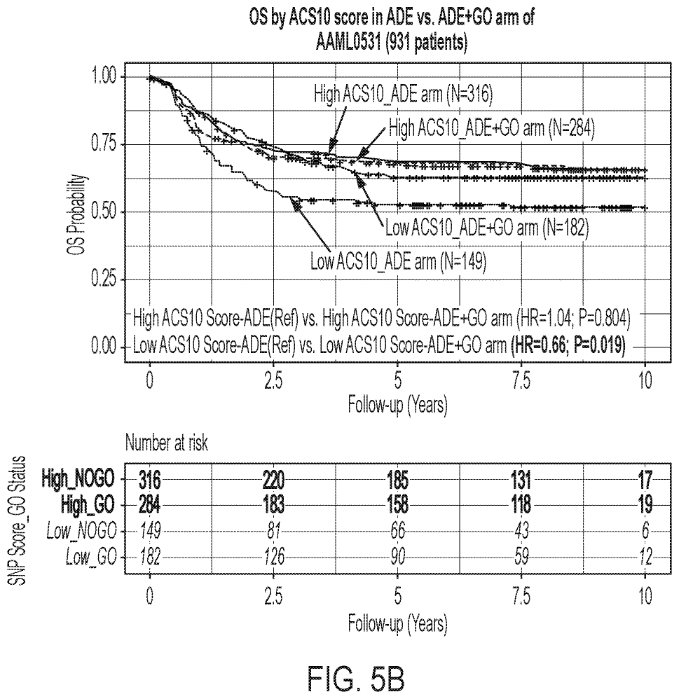

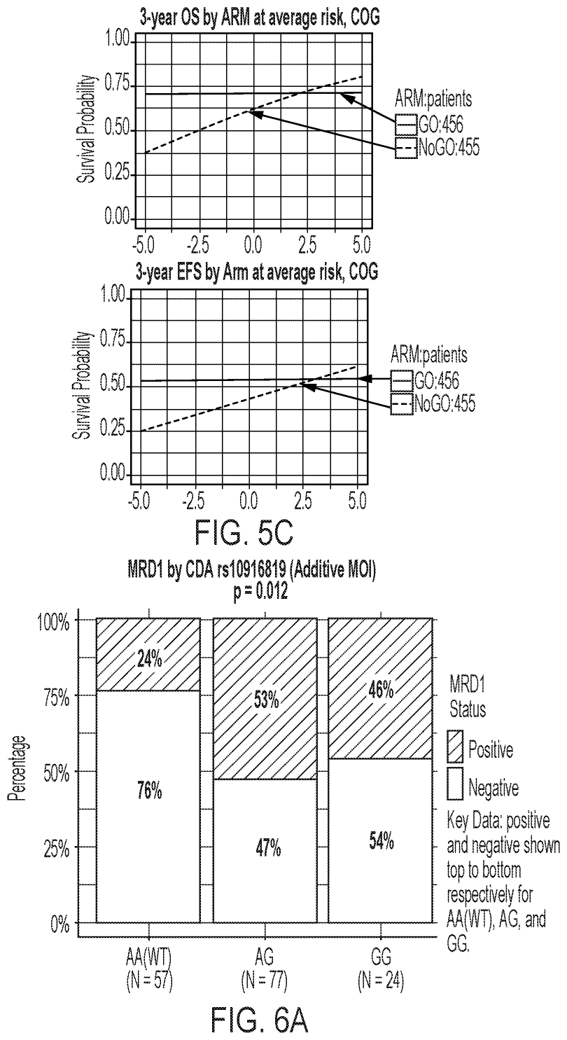

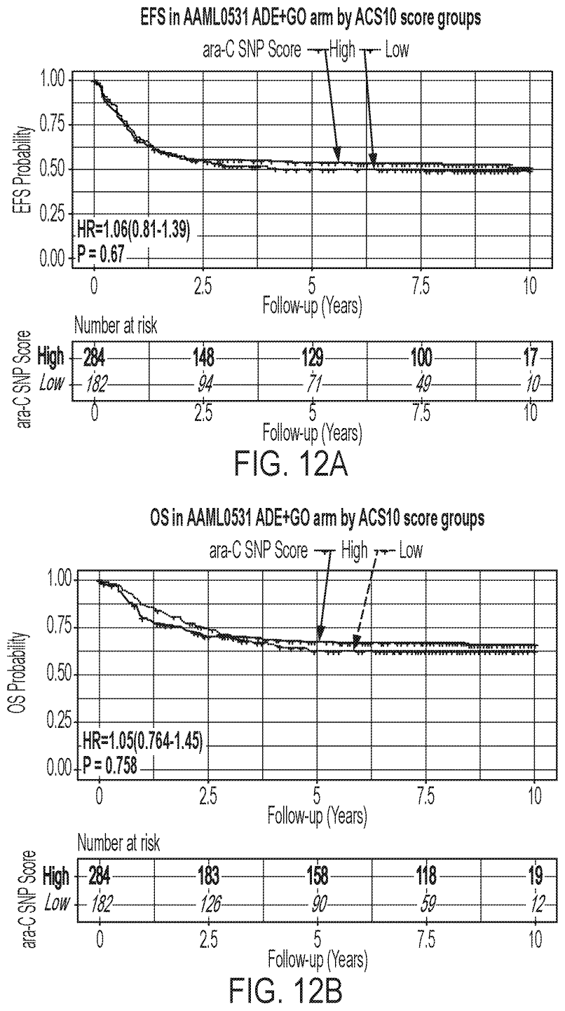

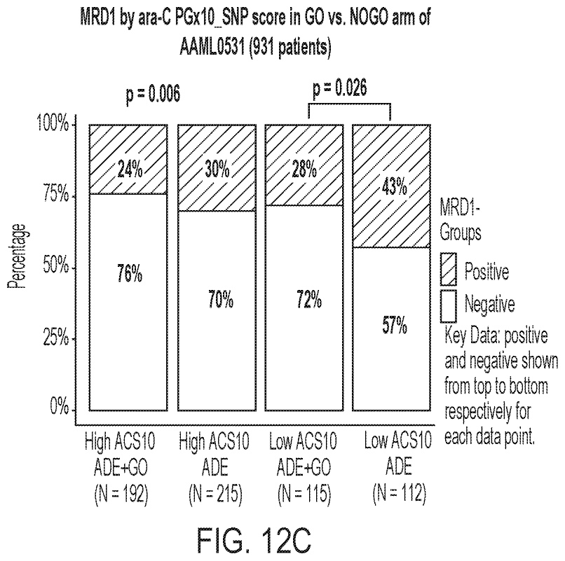

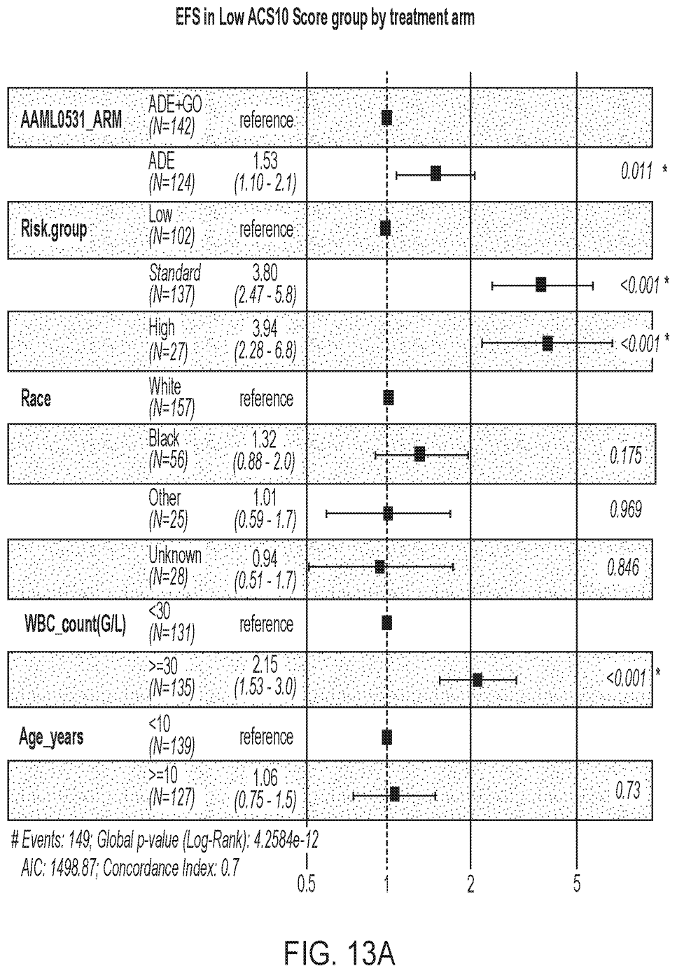

AAML0531 being a randomized study of standard (ADE) arm and standard chemotherapy with addition of GO (ADE+GO), provided a unique opportunity to evaluate whether ACS10 score can provide more insight into treatment regimens when GO was added to the standard chemotherapy. Within ADE+GO arm (n=466), no difference in outcome between those with low-ACS10 (≤0) and those with high-ACS10 (>0) was observed (EFS: HR=1.06, 95% CI=0.81-1.39, p=0.67; OS: HR=1.05, 95% CI-0.76-1.45, p=0.76; and MRD1 p=0.537; FIGS. 12A-12B). Moreover, comparison of treatment arms by ACS10 score groups revealed that low-ACS10 score patients experience improved outcomes with the addition of GO (ADE vs. ADE+GO arms: EFS; HR=0.72, 95% CI=0.534-0.962, P=0.026 and OS; HR=0.66, 95% CI=0.473-0.934, P=0.019, FIGS. 5A and 5B). In contrast, no difference in outcome was observed between ADE and ADE+GO arms for high-ACS10 score patients (EFS: HR=0.91, 95% CI=0.722-1.15, P=0.43; OS: HR=1.04, 95% CI=0.965-1.38, P=0.804, FIGS. 5A and 5B).

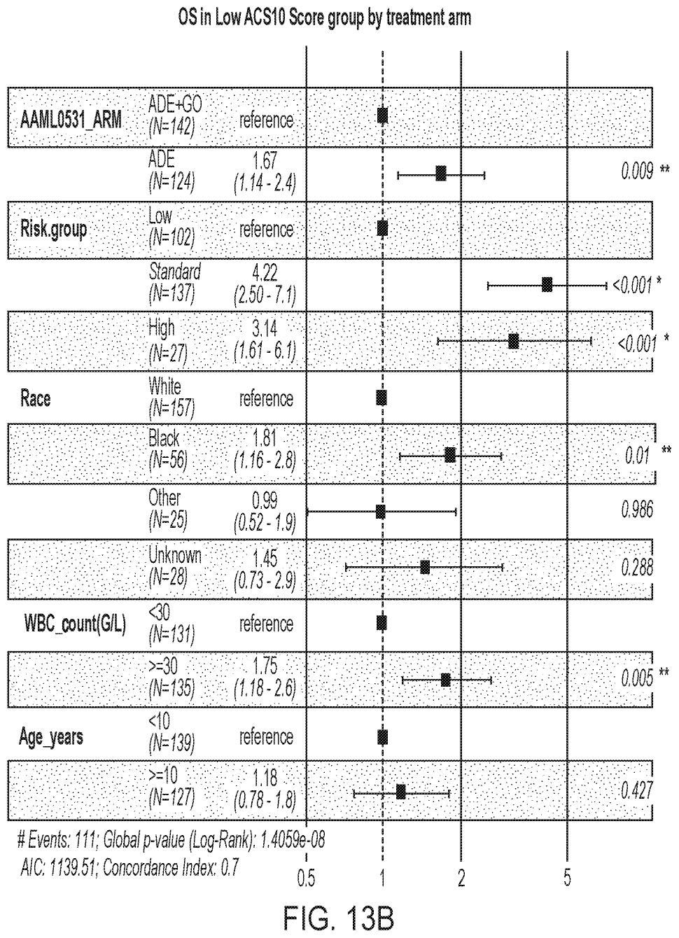

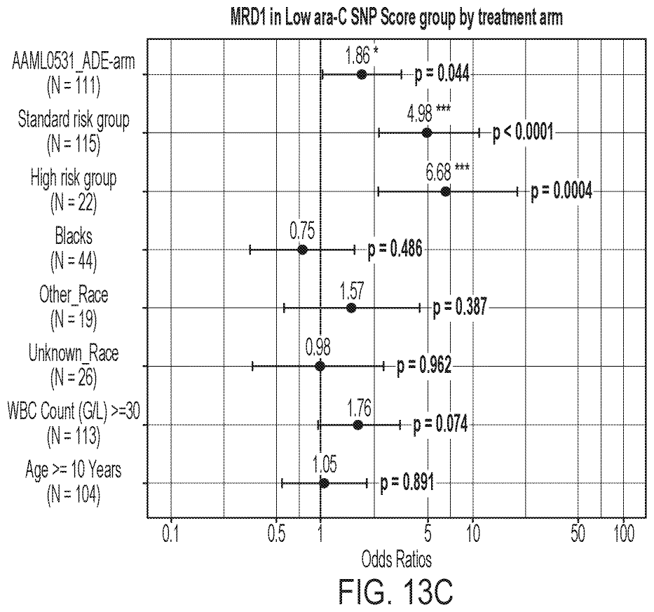

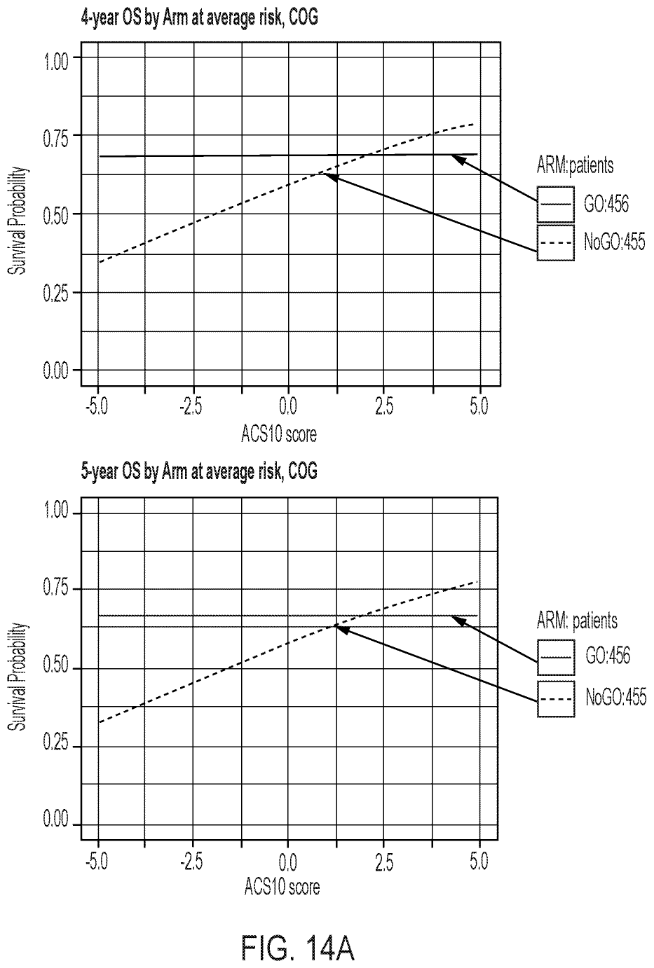

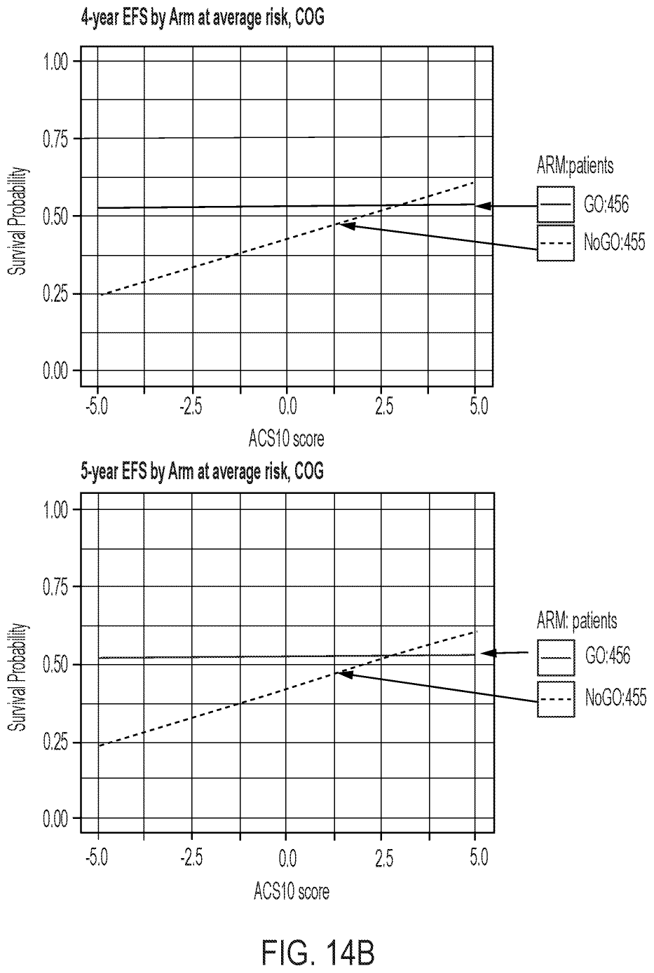

Distribution across both arms and score groups for MRD1 is shown in FIG. 12C). The benefit of GO for low-ACS10 patients retained its statistical significance in a multivariable analysis adjusting for risk group, race, WBC, count, and age (FIGS. 13A-13C). In that analysis, low-ACS10 patients when given ADE+GO experienced events at 0.65 (95% CI=0.47-0.91; p=0.011) and death at 0.60 (95% CI=0.42-0.88; p=0.009) times the rate of those given ADE. Similar to AML02 cohort, in the AAML0531-validation cohort, risk-adjusted Cox model found increasing ACS10 (as a numeric score) was significantly associated with better EFS on the ADE-arm (HR=0.89; 95% CI: 0.82-0.97, p=0.0069) but not on the augmented ADE+GO-arm (HR=0.99; 95% CI: 0.91-1.08, P=0.90). A risk-adjusted Cox model found a significant interaction of ACS10 (as a numeric score) and treatment arm for OS (p=0.032) and a marginally significant interaction for EFS (p=0.075), FIG. 5C. The model estimates that patients with ACS10 score=−3 have three-year EFS (OS) of 53.3% (70.5%) with ADE+GO and 25.2% (37.3%) with ADE and that patients with ACS10 score=+5 have three-year EFS (OS) of 54.6% (71.0%) with ADE+GO and 61.2% (80.4%) with ADE (FIG. 5C). A similar pattern is seen in four and five year patterns (FIGS. 14A-14B and Table 11).

Overall in both cohorts, patients with low-ACS10 reflecting lower ara-CTP levels, had better outcomes with augmented therapy (AML02-HDAC or AAML0531-ADE+GO arms) than with standard therapy (AML02-LDAC or AAML0531-ADE arms).

In addition, it was previously reported that CD33 splicing SNP and a 6 SNP based Pharmacogenomic score for CD33-CD33-PGx6 to be predictive of clinical response to Gemtuzumab. Gemtuzumab ozogamicin (GO) is an immunoconjugate between an anti-CD33 antibody (hP67.6) and a cytotoxin (calicheamicin). A splicing single nucleotide polymorphism (SNP) was reported in CD33 rs12459419 (C>T), resulting in a shorter isoform of CD33 that lacks exon 2 (CD33-D2). Lack of exon 2 results in loss of the IgV domain within the CD33 protein. This is clinically relevant, because the IgV domain is recognized by GO and other CD33 antibodies used in clinical immunophenotyping of AML specimens. Results showed significant association of rs12459419 with diagnostic leukemic cell surface CD33 intensity (determined using IgV targeting p67.6 antibody), as well as differential response in ADE+GO versus ADE treatment arms. Specifically, patients with at least one copy of the variant T allele (CT/TT genotypes) derived no benefit from addition of GO. In contrast, patients with homozygous CC genotype showed significantly better survival (event-free survival [EFS] and disease-free survival [DFS]) as well as lower risk of relapse with the addition of GO to standard chemotherapy. This example was further expanded to include multiple SNPs in CD33 that were associated with clinical outcome, and developed a composite CD33_PGx6_score derived from the six prognostically informative CD33 SNPs-including the splicing SNP-rs1245419. Patients with a CD33_PGx6_score of 0 or higher had higher CD33 expression levels compared with patients with a score of <0 and demonstrated improved disease-free survival and reduced risk of relapse when given ADE+GO as compared to ADE alone in patients treated on AAML0531 clinical trial. No improvement from GO was observed in patients with a CD33_PGx6_score of less than 0. These results hold promise for developing strategies to personalize the use of CD33-directed agents such as GO using CD33 genetics. Use of multiple SNPs in a CD33 comprehensive score allows for global application of the CD33 SNP score in different ethnic groups.

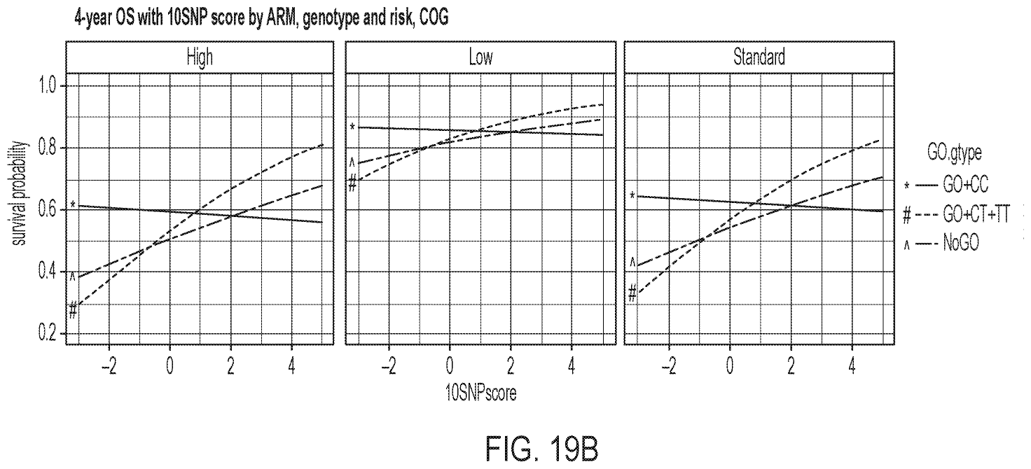

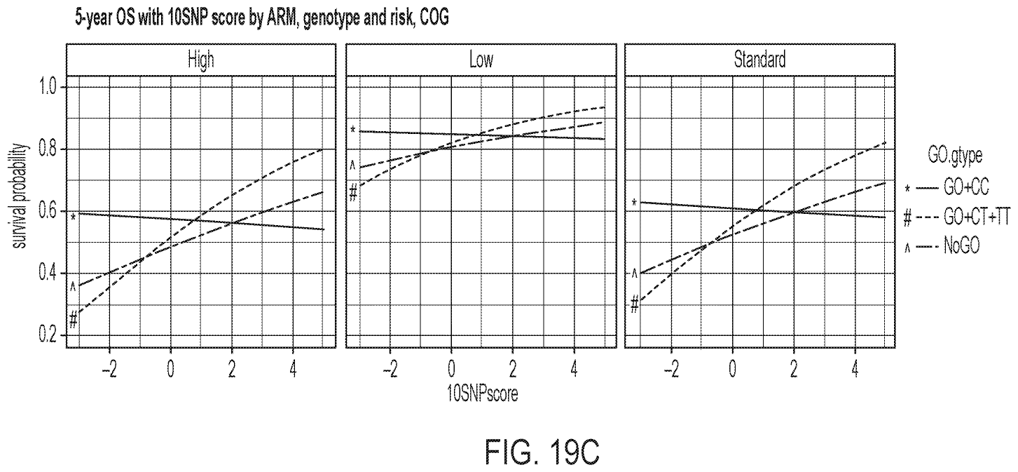

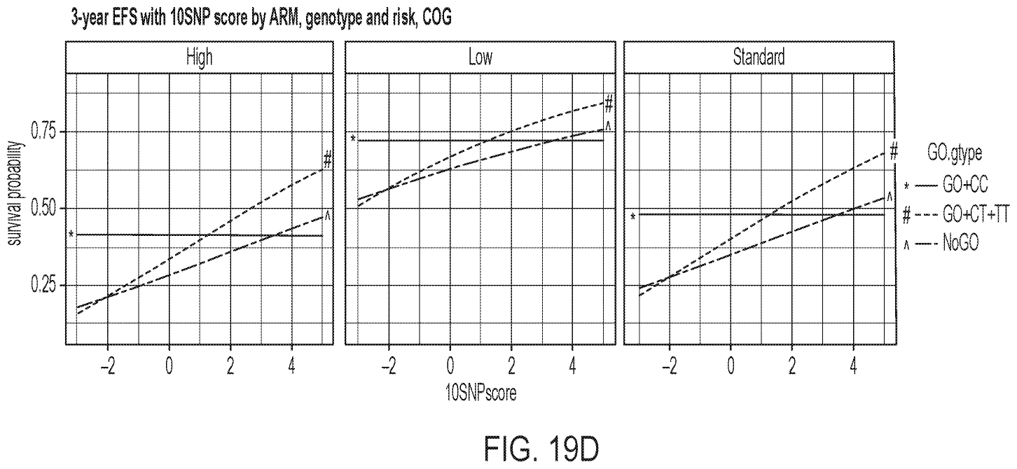

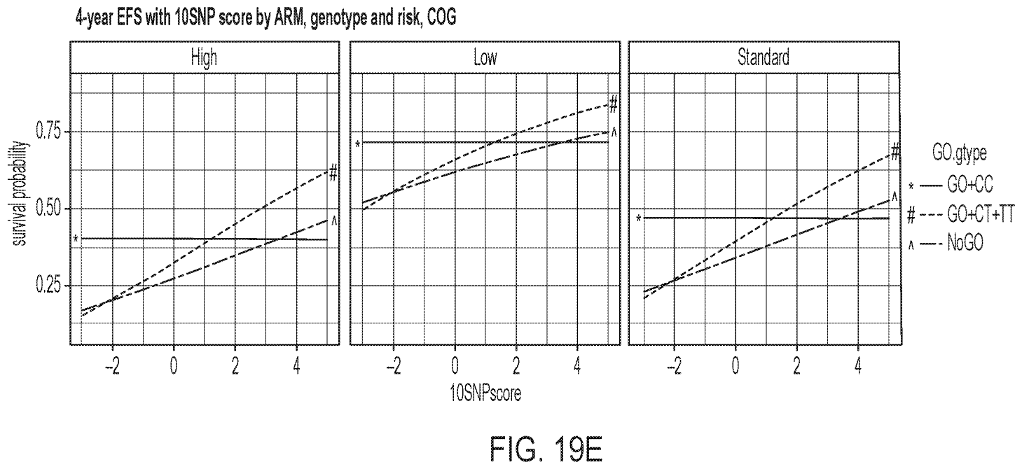

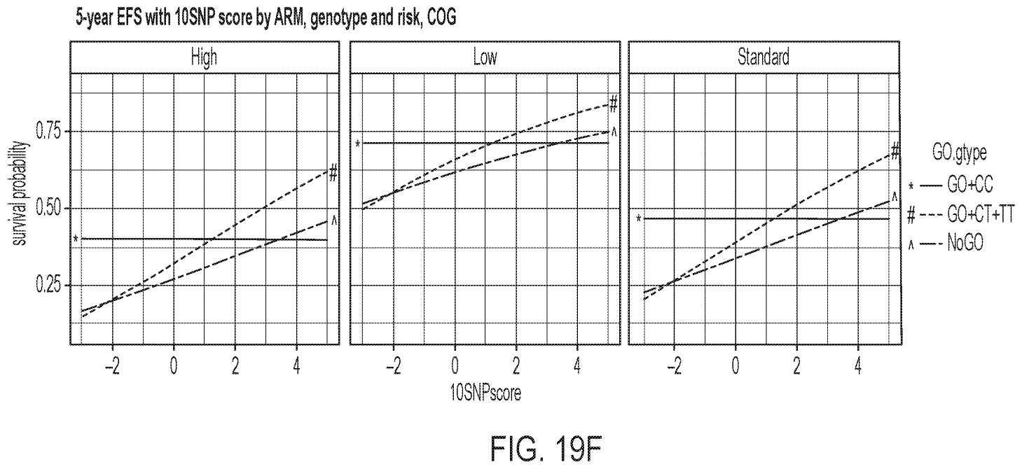

However, given that Gemtuzumab is given in combination with cytarabine containing regimens, the interaction between the CD33 SNP and ACS10 score was further investigated in patients treated with GO. As shown in FIGS. 19A-19F, impact of CD33 splicing SNP and ACS10 SNP score on clinical response in patients treated with ADE+GO or ADE alone in AAML0531 clinical trial. As is depicted in this figure patients treated on ADE alone have poor outcome when they have low ACS10 score. Interestingly, among patients who received GO only those patients that have favorable genotype for CD33 splicing SNP (rs12459419 CC genotype) have better EFS and OS that is not dependent on ACS10 score. However, patients CT or TT genotype patients for CD33 splicing SNP within the low-ACS10 group do not show any benefit from adding GO. Overall the CD33 SNP and ACS10 interaction results show that: i) if patients have low ACS10 score they benefit from adding GO ONLY if they have CD33 rs12459419 CC genotype (FIGS. 19A-19F) (or CD33_PGx6_score 0 or higher). ii) if patients have low ACS10 score and CT/TT genotype for CD33rs12459419 (or CD33_PGx6_score<0) they do not benefit from addition of GO. iii) among patients with high ACS10 score, standard ADE based chemotherapy shows significant improvement in outcome. These results provide a very strong rationale to use both ACS10 and CD33 SNP (score) for designing therapy for most effective treatment in AML.

### DISCUSSION

Cytarabine based regimens have been the mainstay of AML therapy for more than five decades and are likely to remain the backbone of therapy in coming years despite approval of new agents over the past few years as these new agents are primarily given in sequence or in combination with ara-C with or without anthracyclines. Thus, efforts to determine how to best incorporate newly approved agents into clinical care by genomically guided stratification of patients who are more or less likely to benefit can have a significant impact in AML treatment strategies. SNPs of potential relevance have been identified by evaluation of individual genes within metabolic pathway of ara-C activation to ara-CTP. A regression model was recently used to develop an ara-CTP pathway SNP score predictive of leukemic intracellular levels of ara-CTP, suggesting a cumulative or synergistic effect of the SNPs. In the current study, the comprehensive evaluation of 16 key ara-C pathway genes was expanded in a patients from St. Jude AML02 and COG-AAML0531 clinical trials. Univariate evaluation identified several SNPs predictive of one or more clinical endpoints. Testing possible combinations of up to three SNP combinations (computational limitations restricted 3 SNP combination evaluations) in multivariable logistic and Cox regression models identified top models for MRD and EFS with 3 unique SNPs each. Six newly identified SNPs were combined with the four SNPs of previously defined ara-CTP SNP score and developed a comprehensive and robust SNP score of 10 SNPs (ACS10) that captures top SNP combinations of clinical relevance.

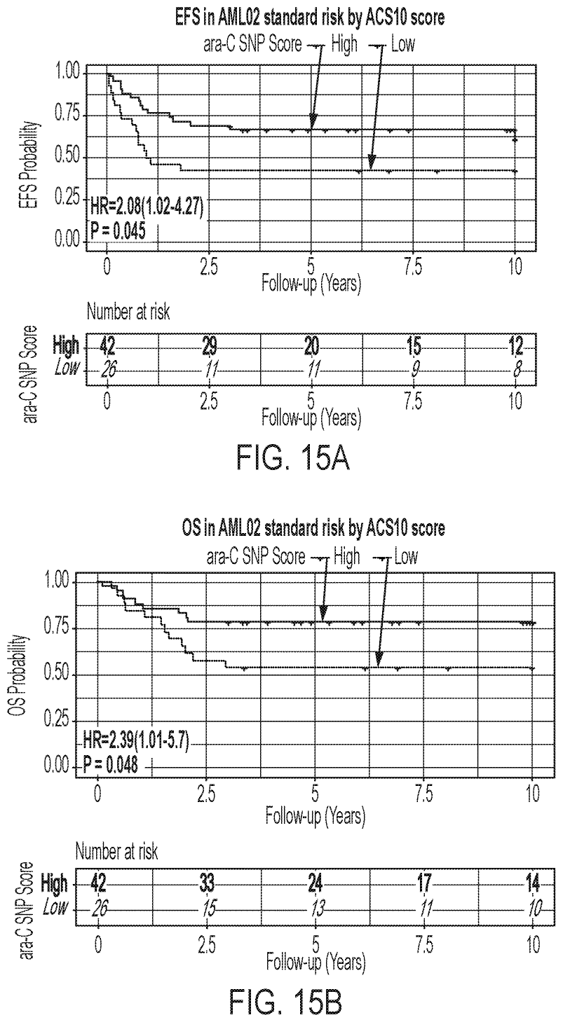

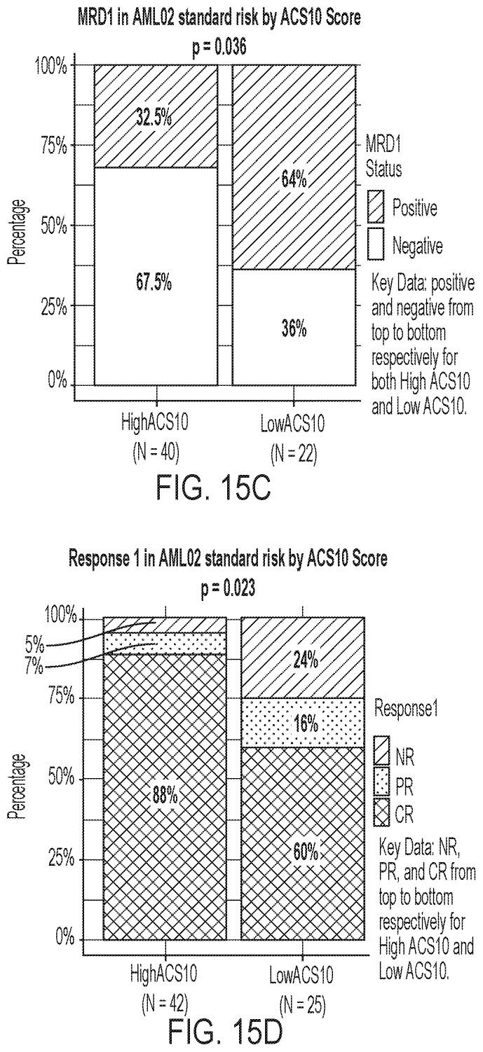

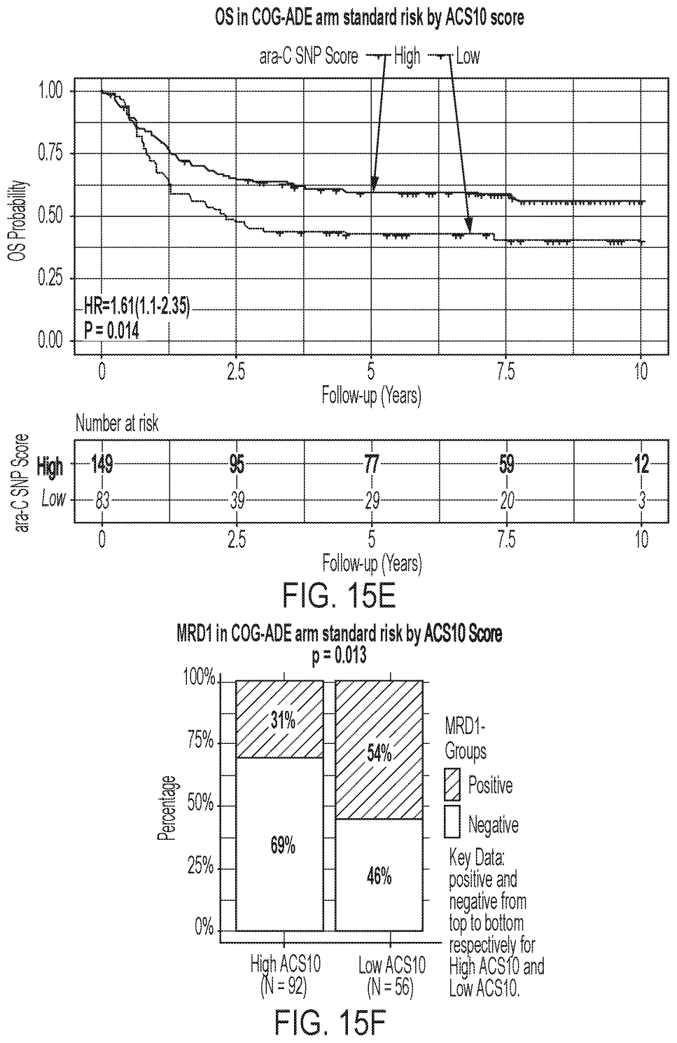

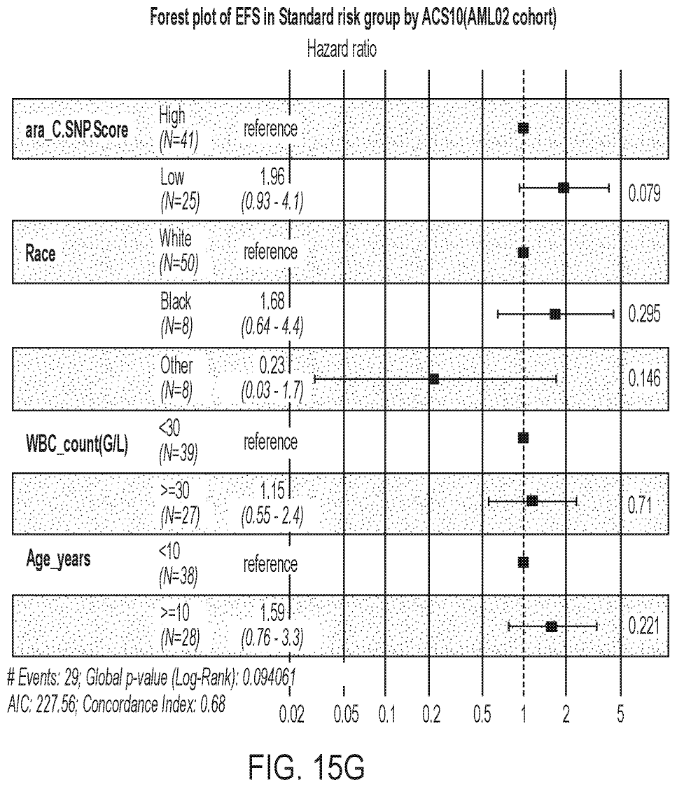

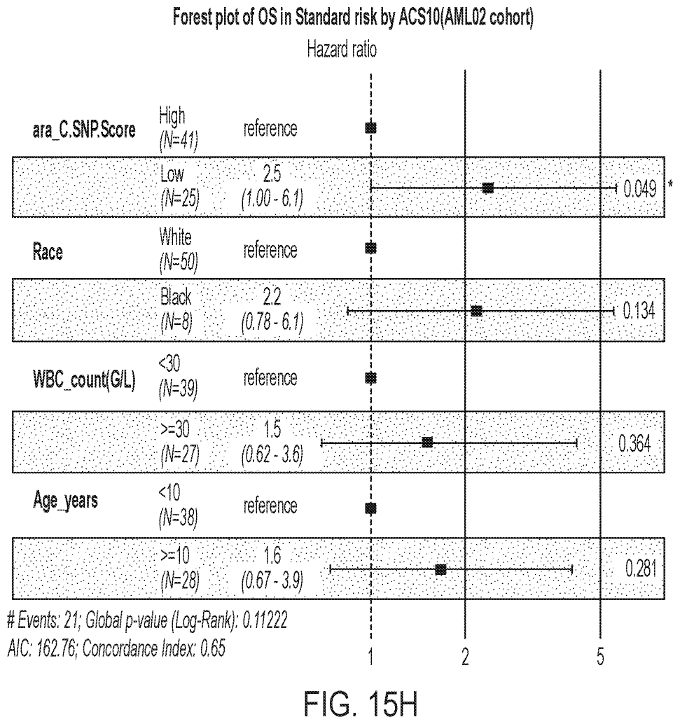

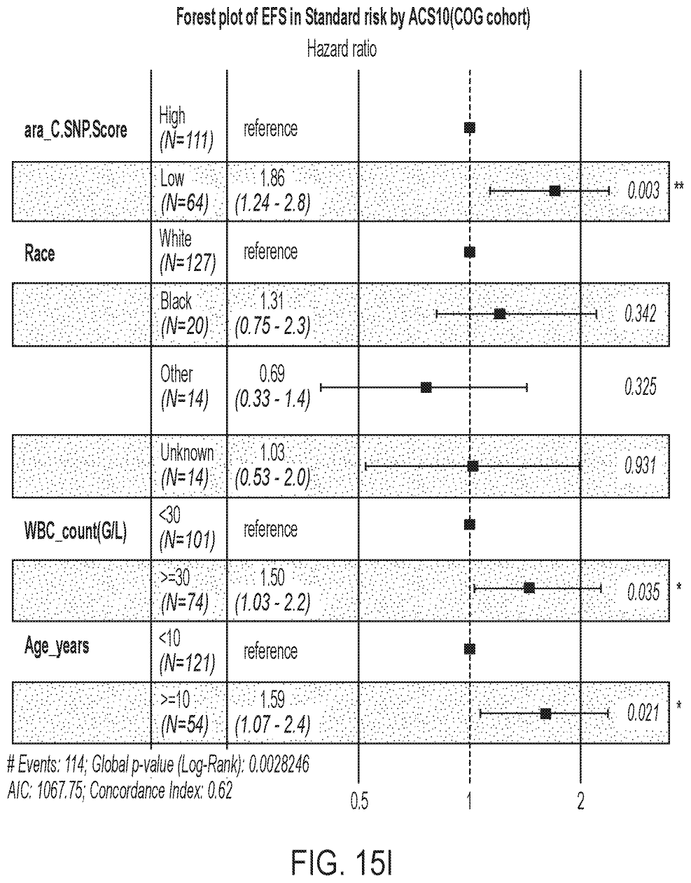

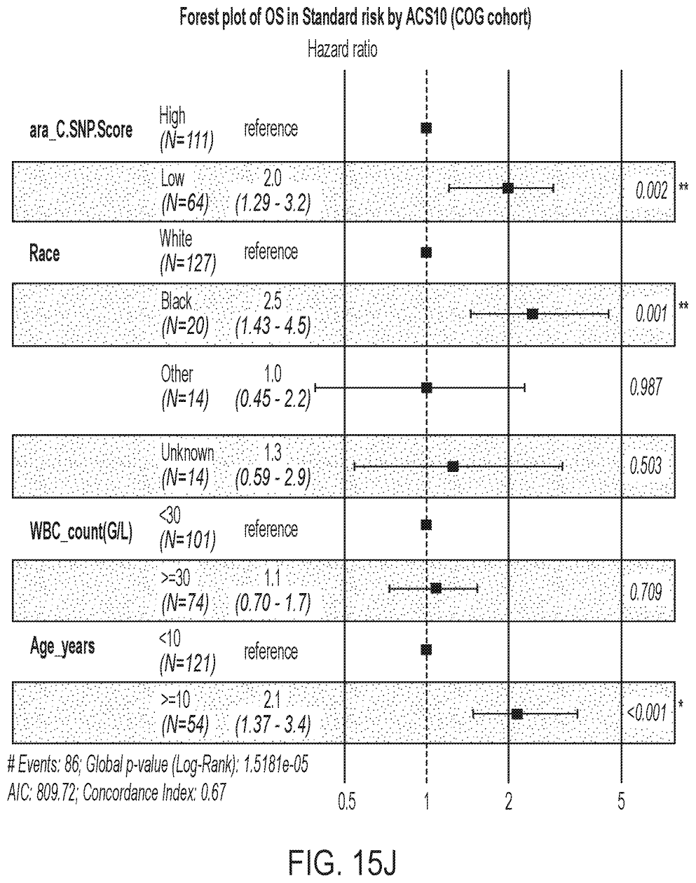

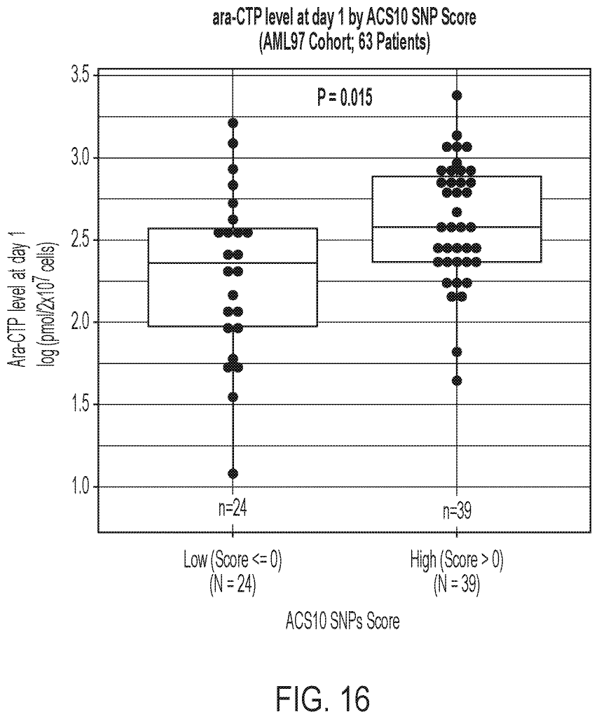

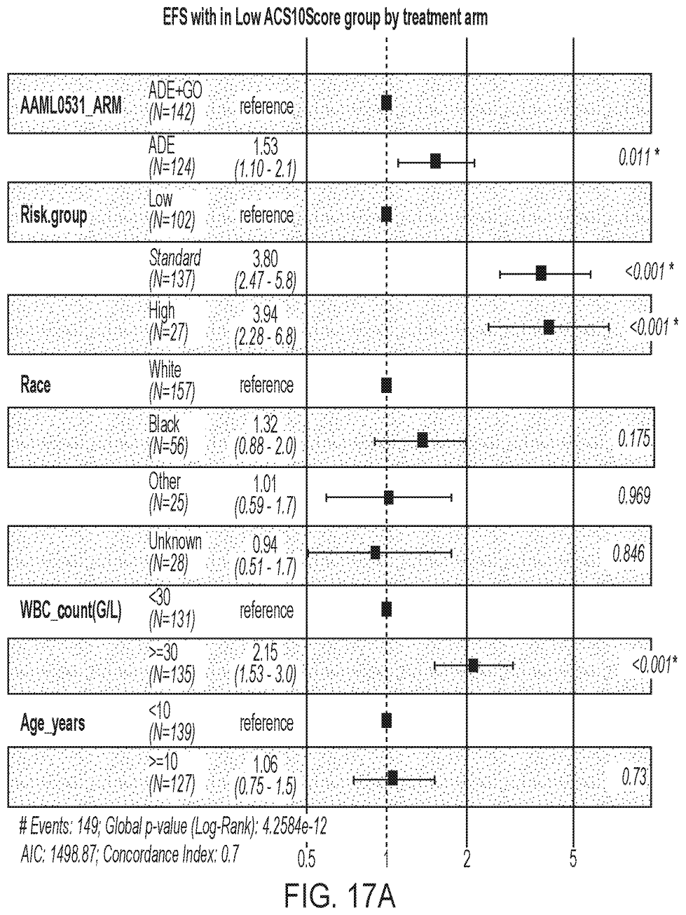

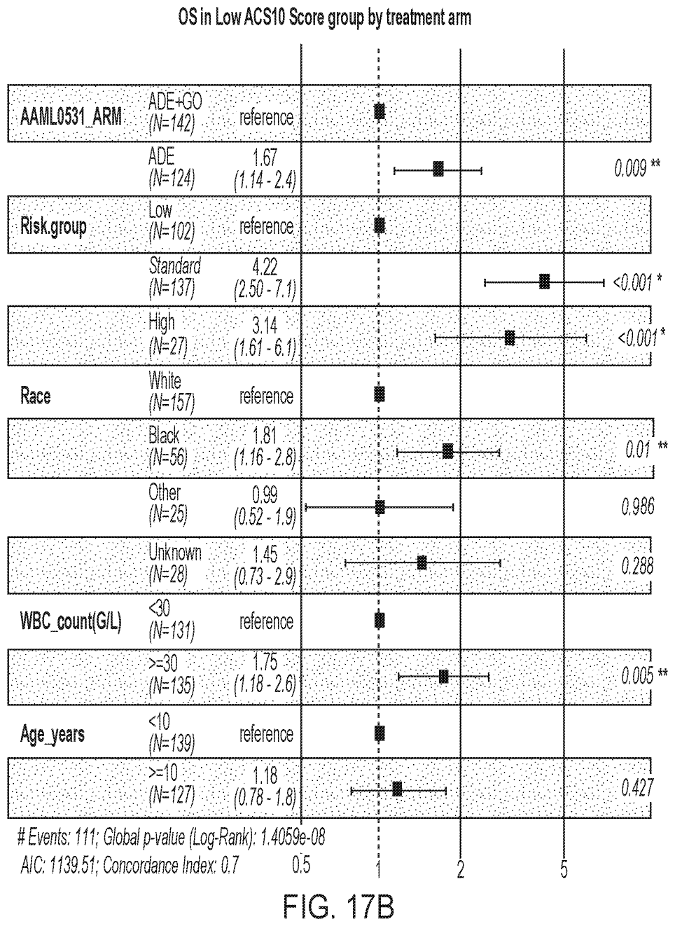

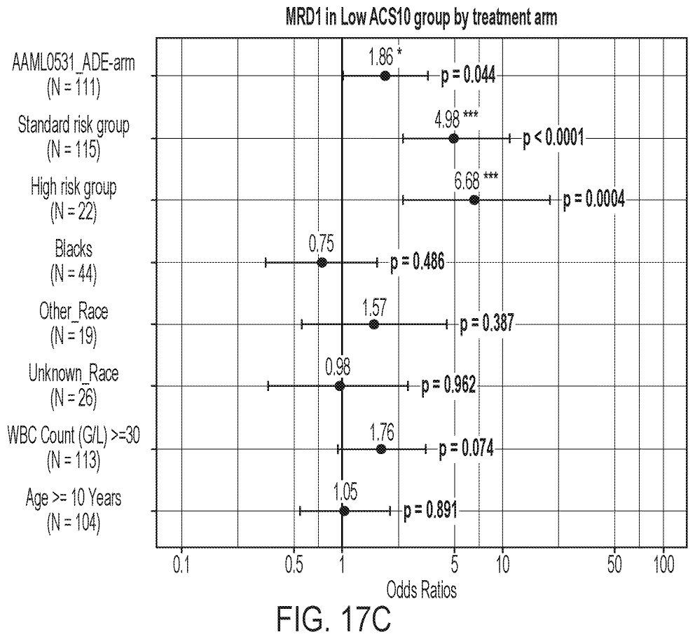

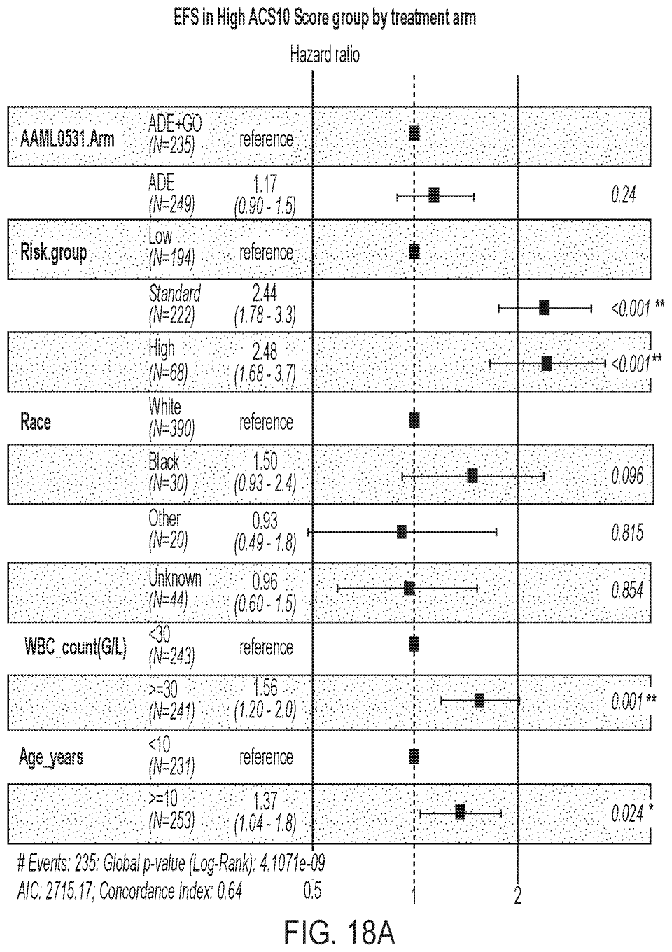

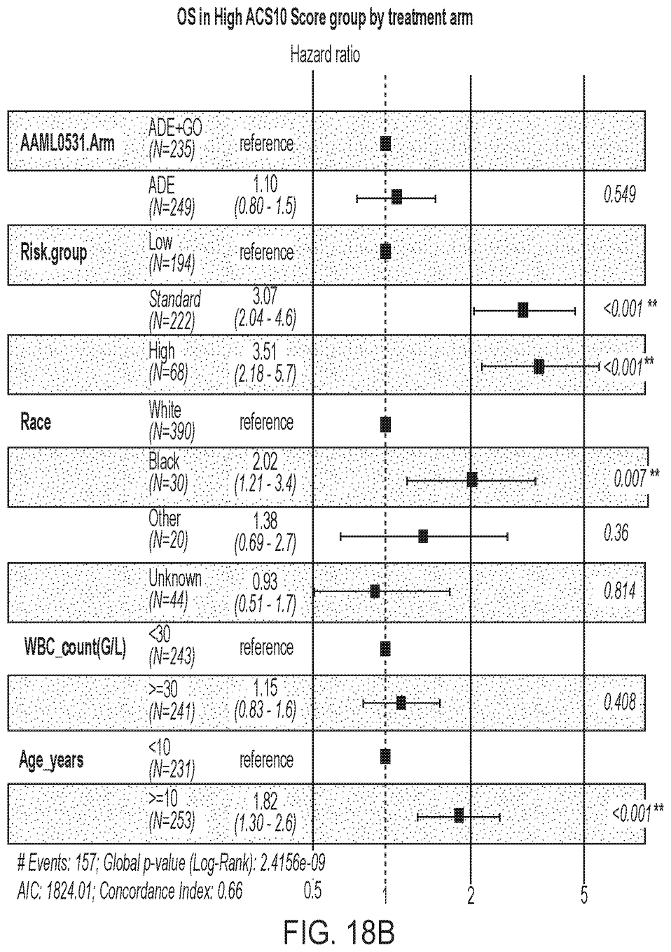

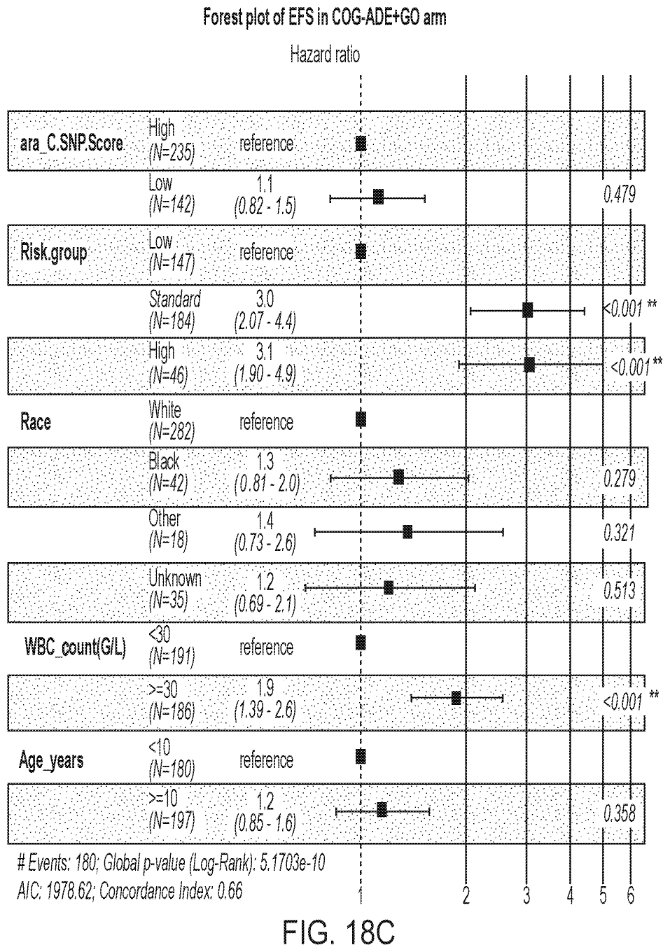

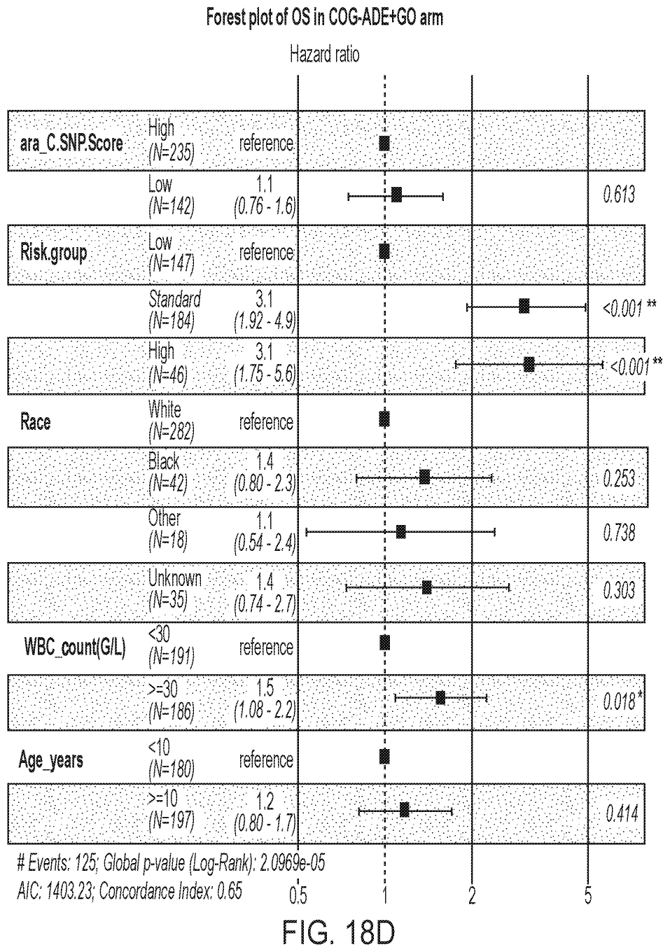

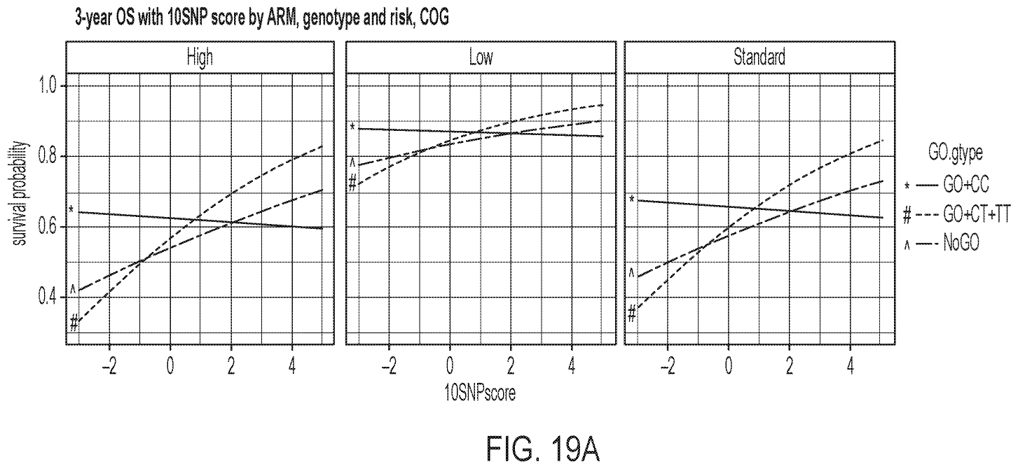

Overall, the findings from this example reveal that: i) Comprehensive pharmacogenomics evaluation of ara-C metabolic pathway genes is more informative as compared to previous studies focused on single genes; ii) ACS10 score groups capture a robust SNP combination signature that is a significant predictor of patients with poor outcome when treated with ara-C based chemotherapy in two independent cohorts tested; iii) Interestingly, greater abundance of the Low-ACS10 score was observed in black patients in both AML02 (33% white patients vs. 73% black patients had low-ACSS score) and COG cohorts (29% white patients vs. 67% black patients had low-ACSS score). Of the 10 SNPs, 3 SNPs contribute towards this racial differences and includes a DCK SNP-rs4643786 with detrimental impact that is more abundant in black patients (variant allele frequency 0.038 vs. 0.48 in white vs. black patients) and SNPs within CMPK1 (rs1044457) and SLC28A3 (rs17343066) with beneficial impact that are less abundant in the black patients (variant allele frequency 0.5 vs. 0.11 and 0.53 vs. 0.15 in white vs. black patients, respectively). This difference in prevalence of the ACS10 score is consistent with historical observations across different studies showing black patients to have worse outcomes as compared to white patients. Though validation in bigger cohorts is required, greater proportion of low-ACS10 SNP score patients within this group might be contributing to the observed racial disparity in outcome; iv) As standard risk group shows high variability in outcome despite lacking the low and high risk group features, performance of ACS10 score was evaluated within this group specifically. ACS10 score groups holds its significant value within standard risk group patients in St. Jude AML02 and COG cohorts thus opening up strategies for stratification of this challenging cohort (FIGS. 15A-15J).; v) ACS10 score expands classification of patients beyond MRD1 stratification to predict outcome and design downstream treatment strategies. vi) Finally, given the availability of AML02 and AAML0531 as randomized studies with low vs. high dose ara C in AML02 or standard ADE vs ADE+GO randomization in AAML0531, ACS10 score was evaluated by treatment arms. Results revealed that within the AML02 cohort, low-ACS10 score was a significant predictor of poor outcome as compared to high-ACS10 score in the LDAC arm, however such similar difference was not observed in the HDAC arm. This observation suggests that patients with low-ACS10 score benefit when given high dose of ara-C, warranting future exploration of pharmacogenetics guided studies to define ara-C dosc. Additionally, patients within high-ACS10 score show similar outcome in LDAC and HDAC arms. This implies that patients with high-ACS10 group might be treated with LDAC to reduce the risk of toxicity without compromising efficacy; vi) AAML0531 cohort randomized patients to standard chemotherapy without (ADE) or with addition of GO (ADE+GO). Within this group patients with low-ACS10 score demonstrated improvement in outcome with addition of GO, suggesting an alternate treatment strategy for these patients.

In both cohorts, patients with low-ACS10 (≤0) had better outcomes with augmented therapy (HDAC or ADE+GO) than with standard therapy (LDAC or ADE). For low ACS10 score patients, the 5 year EFS of was 42% (95% CI=29%, 61%) vs. on AML02 LDAC vs. 55% (95% CI=40%, 75%) on AML02 HDAC arms, similarly 5-year EFS was 40% (95% CI=33%, 49%) on AAML0531-ADE, and 50% (95% CI=43%, 58%) on AAML0531 ADE+GO. Similarly, the five-year OS for these patients was 57% (95% CI: 44%-76%) with LDAC, 63% (95% CI: 48%-83%) with HDAC, 52% (95% CI: 45%, 62%) without GO, and 63% (95% CI: 56%, 72%) with GO.

In conclusion, results using a comprehensive pharmacogenomic evaluation of the pathway and regression modeling approach not only provided a unique ACS10 score of prognostic significance that can predict poor outcome in AML, but suggested that alternative treatment strategies with either high dose ara-C or addition of GO are more suitable strategies for patients with detrimental low-ACS10 score. Further validation of this score especially in context of the alternative therapeutic options as suggested by current evaluation or combination with other newly approved agents such as glasdegib or venetoclax is needed to improve precision medicine in AML.

### Example 2

As is depicted in FIGS. 19A-19F, patients treated on ADE alone have poor outcome when they have low ACS10 score (as described previously). Interestingly among patients who received GO only those patients that have favorable genotype for CD33 splicing SNP do better and the outcome is no dependent on ACS10 score. However, patients with CT or TT genotype for CD33 splicing SNP within in low-ACS10 group do not show any benefit from adding GO. These results provide a very strong rationale to use both ACS10 and CD33 SNP (score) for designing therapy for most effective treatment in AML.

### Example 3

A comprehensive pharmacogenomics evaluation of SNPs in ara-C metabolism pathway genes was performed and a polygenic ara-C 10-SNP genotype (ACS10) score based on association with leukemic intracellular ara-CTP levels and clinical outcome was developed, as described in Examples 1 and 2. Data indicates that patients with low ACS10 score had poor outcomes when given a standard induction including low-dose ara-C, daunorubicin and etoposide (ADE) but their outcomes were much better by augmenting that therapy with either an increased dose of ara-C or the addition of gemtuzumab ozogamicin (GO). Conversely, patients with a high ACS10 score fared worse when given high-dose ara-C than low ara-C dose-based ADE induction (LDAC).

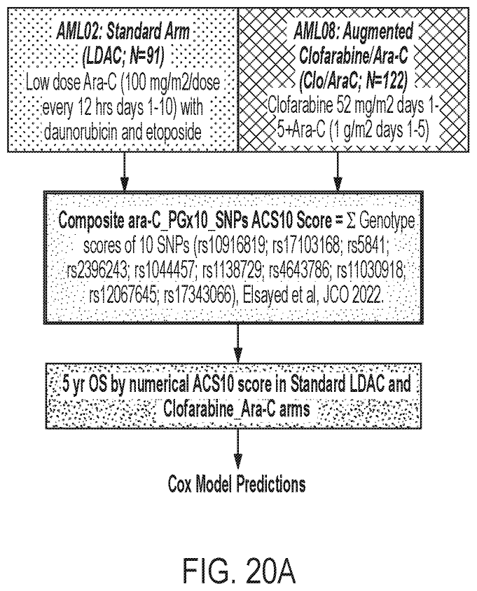

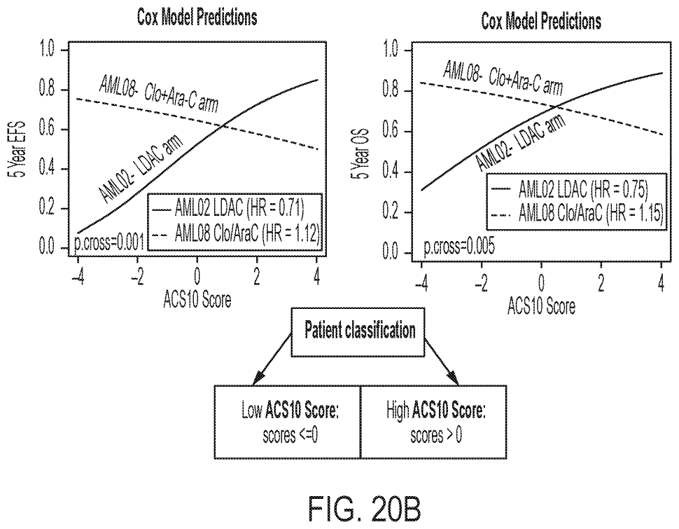

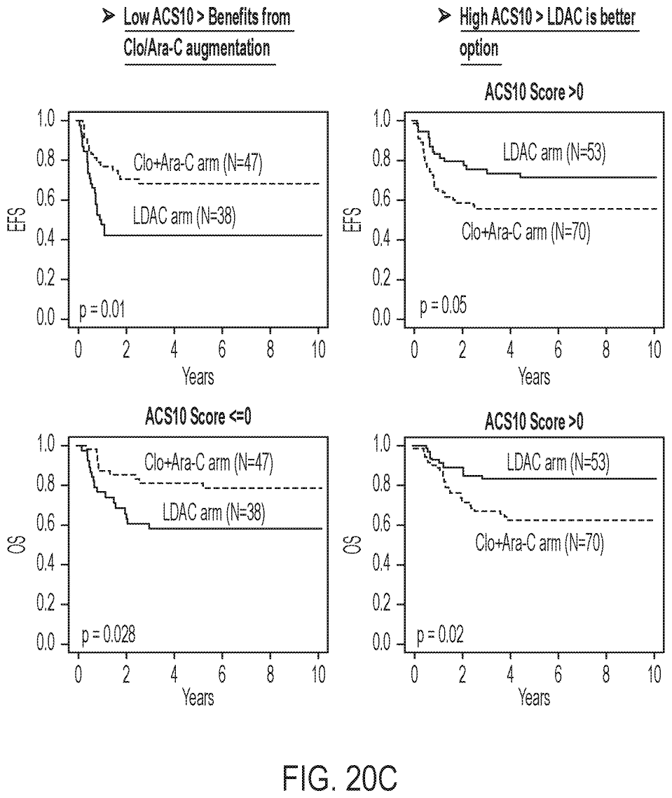

Clofarabine is another nucleoside analog that inhibits both DNA polymerase as well as ribonucleotide reductase and thus can enhance the activity of ara-C. The association of ACS10 score with outcomes among 91 patients randomly assigned standard ADE induction on the AML02 clinical trial (LDAC arm) (NCT00136084) and 117 patients randomly assigned an induction of clofarabine and ara-C(Clo+Ara-C arm) on the AML08 clinical trial (NCT00703820). FIG. 20A shows a schematic of the study design described in this example. Briefly, 5-year event-free survival (EFS) and overall survival (OS) of patients treated with ADE improved with increasing ACS10 score. However, for patients treated with Clo+Ara-C, improvement with increasing ACS10 score was not observed (FIG. 20B). This differential outcome association pattern indicates that thresholding the ACS10 score at 0 can be used to classify patients into low (<=0) and high (>0) ACS10 groups. Patients within low ACS10 score group had a better outcome with Clo+araC and a high ACS10 score patient group fared better with ADE. In particular, there is significant improvement in EFS and OS of the low-ACS10 score patients when treated with Clo+Ara-C as compared to ADE (Clo+araC vs. ADE; EFS HR=0.45, 95% CI: 0.23-0.82; p=0.01; OS HR-0.45, 95% CI: 0.19-0.93, p=0.03; FIG. 20C). In contrast, patients with a high ACS10 score had worse EFS and OS with Clo+araC induction than with ADE induction (EFS HR=1.84, 95% CI: 0.99-3.41, p=0.05; OS HR=2.40, 95% CI: 1.13-5.13; p=0.02, FIG. 20C).

The observations described above are unlikely to suffer from a strong historical comparison bias because there are not statistically significant differences in the outcomes of the randomly assigned therapies of the AML02 and AML08 trials, as well as outcome by the clinical trial. For example, the EFS/OS of each arm of the two trials, as well as between AML02 and AML08 trials was p=0.8.

In summary, this data indicates that the outcome of patients with Low-ACS10 genetic score prognosis is poor with standard ADE induction. However, therapy augmentation options such as Clo+Ara-C, ADE with high-dose ara-C, and ADE+gemtuzumab may improve the prognosis.

TABLE 2
SNPs associated with multiple clinical outcomes in patients from St. Jude AML02 cohort

| | | | | | | | Unadjusted association analysis | | | | | | Risk Adjusted Evaluation | | | | | |
|---|---|---|---|---|---|---|---|---|---|---|---|---|---|---|---|---|---|---|
| | | | | | | | MRD1 | | EFS | | OS | | MRD1 | | EFS | | OS | |
| | | | | | | | OR(95% | | HR(95% | P- | HR(95% | P- | OR(95% | P- | HR(95% | P- | HR(95% | P- |
| SNP | WT | VAR | Gene | Position | MAF | MOI | CI) | P-value | CI) | value | CI) | value | CI) | value | CI) | value | CI) | value |
| rs10916819 | A | G | CDA | 14 kb 5′ | 0.391 | D | 1.81 | 0.001 | 1.37 | 0.025 | 1.61 | 0.011 | 1.86 | 0.002 | 1.37 | 0.026 | 1.63 | 0.009 |
| | | | | | | | (1.26-2.59) | | (1.04-1.8) | | (1.12-2.31) | | (1.25-2.77) | | (1.04-1.8) | | (1.13-2.35) | |
| rs1048977 | C | T | CDA | Synonymous | 0.288 | R | 3.51 | 0.019 | 1.08 | 0.754 | 1.01 | 0.971 | 4.36 | 0.01 | 1.07 | 0.793 | 0.98 | 0.953 |
| | | | | | | | (1.23-10.07) | | (0.65-1.79) | | (0.56-1.59) | | (1.42-13.42) | | (0.65-1.77) | | (0.55-1.77) | |
| rs2072671 | A | C | CDA | Missense | 0.296 | R | 0.55 | 0.041 | 0.6 | 0.051 | 0.54 | 0.089 | 0.49 | 0.014 | 0.7 | 0.061 | 0.67 | 0.083 |
| | | | | | | | (0.31-0.98) | | (0.36-1.01) | | (0.27-1.1) | | (0.28-0.87) | | (0.49-1.02) | | (0.42-1.05) | |
| rs818196 | G | C | CDA | Intronic | 0.223 | R | 1.7 | 0.226 | 1.58 | 0.048 | 1.88 | 0.007 | 1.96 | 0.184 | 1.46 | 0.11 | 1.75 | 0.019 |
| | | | | | | | (0.72-4.04) | | (1.01-2.49) | | (1.18-3) | | (0.73-5.28) | | (0.92-2.31) | | (1.1-2.78) | |
| rs580032 | T | G | CDA | Intronic | 0.0903 | D | 1.01 | 0.952 | 0.64 | 0.034 | 0.83 | 0.379 | 1.03 | 0.917 | 0.61 | 0.023 | 0.81 | 0.34 |
| | | | | | | | (0.66-1.55) | | (0.42-0.97) | | (0.54-1.27) | | (0.62-1.7) | | (0.4-0.93) | | (0.52-1.25) | |
| rs602950 | T | C | CDA | 5′UTR | 0.2951 | A | 0.61 | 0.0507 | 0.81 | 0.254 | 0.75 | 0.217 | 0.49 | 0.016 | 0.74 | 0.106 | 0.67 | 0.09 |
| | | | | | | | (0.37-1.01) | | (0.56-1.16) | | (0.48-1.18) | | (0.28-0.87) | | (0.51-1.07) | | (0.42-1.07) | |
| rs12404655 | A | G | CDA | Intronic | 0.2259 | R | 1.56 | 0.235 | 0.96 | 0.897 | 0.91 | 0.792 | 2.37 | 0.047 | 1.13 | 0.679 | 1.03 | 0.938 |
| | | | | | | | (0.75-3.25) | | (0.54-1.72) | | (0.45-1.85) | | (1.01-5.52) | | (0.63-2.04) | | (0.51-2.09) | |
| rs17103168 | A | G | CMPK1 | 4 kb 3′ | 0.217 | D | 0.58 | 0.002 | 1 | 0.983 | 0.96 | 0.808 | 0.55 | 0.002 | 1.06 | 0.663 | 1.04 | 0.799 |
| | | | | | | | (0.42-0.82) | | (0.78-1.27) | | (0.72-1.29) | | (0.37-0.8) | | (0.83-1.35) | | (0.77-1.4) | |
| rs3088062 | T | C | CMPK1 | 3′UTR | 0.194 | D | 1.29 | 0.134 | 1.3 | 0.032 | 1.37 | 0.033 | 1.06 | 0.754 | 1.14 | 0.312 | 1.17 | 0.293 |
| | | | | | | | (0.93-1.79) | | (1.02-1.65) | | (1.03-1.82) | | (0.73-1.54) | | (0.88-1.47) | | (0.87-1.58) | |
| rs4600090 | C | A | CMPK1 | Intronic | 0.158 | D | 1.2 | 0.293 | 1.26 | 0.064 | 1.39 | 0.028 | 1.08 | 0.698 | 1.15 | 0.278 | 1.26 | 0.125 |
| | | | | | | | (0.85-1.7) | | (0.99-1.61) | | (1.04-1.86) | | (0.73-1.59) | | (0.89-1.49) | | (0.94-1.7) | |
| rs7543016 | C | G | CMPK1 | Missense | 0.428 | R | 1.66 | 0.018 | 0.79 | 0.18 | 0.66 | 0.083 | 2.02 | 0.006 | 0.82 | 0.284 | 0.67 | 0.095 |
| | | | | | | | (1.09-2.51) | | (0.55-1.12) | | (0.42-1.05) | | (1.23-3.33) | | (0.58-1.17) | | (0.42-1.07) | |
| rs1044457 | C | T | CMPK1 | 3′UTR | 0.404 | A | 1.15 | 0.542 | 0.77 | 0.168 | 0.63 | 0.039 | 1.25 | 0.399 | 0.8 | 0.256 | 0.64 | 0.049 |
| | | | | | | | (0.73-1.8) | | (0.54-1.11) | | (0.41-0.98) | | (0.75-2.07) | | (0.55-1.17) | | (0.41-0.99) | |
| rs3925058 | T | C | CMPK1 | 1.8 kb 5′ | 0.443 | A | 1.09 | 0.716 | 0.61 | 0.007 | 0.63 | 0.036 | 1.18 | 0.545 | 0.63 | 0.014 | 0.66 | 0.062 |
| | | | | | | | (0.68-1.74) | | (0.42-0.87) | | (0.41-0.97) | | (0.69-2.01) | | (0.43-0.91) | | (0.43-1.02) | |
| rs7533657 | C | T | CTPS1 | 12 kb 5′ | 0.288 | R | 1.11 | 0.706 | 1.46 | 0.027 | 1.53 | 0.029 | 1.16 | 0.619 | 1.46 | 0.028 | 1.56 | 0.024 |
| | | | | | | | (0.65-1.89) | | (1.04-2.05) | | (1.04-2.24) | | (0.65-2.08) | | (1.04-2.04) | | (1.06-2.29) | |
| rs4643786 | T | C | DCK | 3′UTR | 0.133 | R | 1.31 | 0.392 | 1.5 | 0.043 | 1.53 | 0.053 | 1.27 | 0.507 | 1.47 | 0.057 | 1.42 | 0.114 |
| | | | | | | | (0.71-2.43) | | (1.01-2.22) | | (0.99-2.36) | | (0.63-2.58) | | (0.99-2.17) | | (0.92-2.19) | |
| rs6850978 | C | T | DCTD | 250 kb 5′ | 0.385 | A | 1.66 | 0.026 | 1.12 | 0.491 | 0.93 | 0.696 | 2.64 | 0.0007 | 1.31 | 0.12 | 1.07 | 0.744 |
| | | | | | | | (1.06-2.6) | | (0.81-1.54) | | (0.63-1.37) | | (1.5-4.65) | | (0.93-1.84) | | (0.71-1.62) | |
| rs6829021 | G | A | DCTD | 1.1 kb 3′ | 0.247 | D | 0.69 | 0.028 | 1.12 | 0.365 | 1.1 | 0.508 | 0.58 | 0.005 | 1.09 | 0.478 | 1.05 | 0.726 |
| | | | | | | | (0.49-0.96) | | (0.88-1.42) | | (0.83-1.47) | | (0.39-0.85) | | (0.86-1.39) | | (0.79-1.4) | |
| rs4742 | T | C | DCTD | Synonymous | 0.332 | R | 1.14 | 0.641 | 1.53 | 0.011 | 1.69 | 0.003 | 1.14 | 0.682 | 1.51 | 0.016 | 1.67 | 0.004 |
| | | | | | | | (0.67-1.94) | | (1.1-2.11) | | (1.19-2.4) | | (0.62-2.09) | | (1.08-2.1) | | (1.17-2.38) | |
| rs6552622 | C | G | DCTD | Intronic | 0.102 | A | 1.14 | 0.682 | 2.03 | 0.002 | 2.27 | 0.0005 | 0.99 | 0.988 | 1.86 | 0.007 | 2.1 | 0.002 |
| | | | | | | | (0.59-2.21) | | (1.29-3.2) | | (1.43-3.61) | | (0.48-2.06) | | (1.18-2.93) | | (1.32-3.33) | |
| rs2037067 | G | A | DCTD | 250 kb 5′ | 0.3571 | R | 1.49 | 0.088 | 1.2 | 0.256 | 1.21 | 0.299 | 2.5 | 0.003 | 1.4 | 0.037 | 1.48 | 0.038 |
| | | | | | | | (0.94-2.36) | | (0.88-1.64) | | (0.84-1.74) | | (1.37-4.54) | | (1.02-1.93) | | (1.02-2.15) | |
| rs5841 | C | T | NME4 | Synonymous | 0.415 | D | 0.59 | 0.002 | 0.91 | 0.421 | 0.98 | 0.905 | 0.65 | 0.024 | 1 | 0.996 | 1.13 | 0.43 |
| | | | | | | | (0.43-0.83) | | (0.71-1.15) | | (0.73-1.32) | | (0.45-0.94) | | (0.78-1.28) | | (0.84-1.52) | |
| rs11598702 | T | C | NT5C2 | Intronic | 0.317 | R | 0.74 | 0.283 | 0.55 | 0.044 | 0.69 | 0.214 | 0.73 | 0.305 | 0.54 | 0.038 | 0.68 | 0.202 |
| | | | | | | | (0.43-1.28) | | (0.31-0.99) | | (0.38-1.24) | | (0.4-1.34) | | (0.3-0.97) | | (0.38-1.23) | |
| rs1712517 | A | G | NT5C2 | 80 kb 3′ | 0.491 | D | 0.84 | 0.352 | 0.83 | 0.183 | 0.7 | 0.019 | 0.7 | 0.105 | 0.75 | 0.038 | 0.59 | 0.001 |
| | | | | | | | (0.58-1.22) | | (0.64-1.09) | | (0.51-0.94) | | (0.45-1.08) | | (0.57-0.98) | | (0.43-0.81) | |
| rs4917384 | C | T | NT5C2 | 40 kb 3′ | 0.2623 | R | 1.69 | 0.235 | 1.38 | 0.217 | 1.52 | 0.161 | 2.4 | 0.094 | 1.64 | 0.064 | 2.09 | 0.019 |
| | | | | | | | (0.71-4.01) | | (0.83-2.82) | | (0.85-2.73) | | (0.86-6.66) | | (0.97-2.75) | | (1.13-3.86) | |
| rs12806698 | C | A | RRM1 | 5′UTR | 0.1951 | D | 1.01 | 0.977 | 0.81 | 0.109 | 0.89 | 0.454 | 0.93 | 0.702 | 0.76 | 0.047 | 0.83 | 0.245 |
| | | | | | | | (0.72-1.4) | | (0.62-1.05) | | (0.65-1.21) | | (0.63-1.36) | | (0.58-0.99) | | (0.6-1.14) | |
| rs1138729 | A | G | RRM2 | 3′UTR | 0.159 | D | 1.13 | 0.543 | 1.35 | 0.019 | 1.41 | 0.023 | 1.06 | 0.757 | 1.34 | 0.022 | 1.4 | 0.029 |
| | | | | | | | (0.78-1.59) | | (1.05-1.73) | | (1.05-1.91) | | (0.71-1.59) | | (1.04-1.72) | | (1.04-1.88) | |
| rs6742272 | C | G | RRM2 | 18 kb 5′ | 0.3253 | A | 0.64 | 0.061 | 1.28 | 0.139 | 1.25 | 0.257 | 0.5 | 0.012 | 1.22 | 0.228 | 1.23 | 0.31 |
| | | | | | | | (0.4-1.02) | | (0.92-1.77) | | (0.85-1.86 | | (0.29-0.86) | | (0.88-1.7) | | (0.83-1.82) | |
| rs3743162 | C | T | SLC28A1 | Intronic | 0.1878 | A | 1.3 | 0.379 | 1.39 | 0.138 | 1.63 | 0.054 | 1.38 | 0.344 | 1.44 | 0.096 | 1.73 | 0.03 |
| | | | | | | | (0.72-2.34) | | (0.9-2.13) | | (0.99-2.67) | | (0.71-2.66) | | (0.94-2.22) | | (1.05-2.85) | |
| rs4980345 | G | A | SLC28A1 | Intronic | 0.1265 | D | 0.79 | 0.271 | 1.17 | 0.272 | 1.3 | 0.102 | 0.89 | 0.604 | 1.22 | 0.153 | 1.4 | 0.037 |
| | | | | | | | (0.53-1.19) | | (0.89-1.54) | | (0.95-1.77) | | (0.57-1.39) | | (0.93-1.61) | | (1.02-1.92) | |
| rs12554292 | C | T | SLC28A3 | 83 kb 5′ | 0.097 | D | 1.53 | 0.043 | 0.92 | 0.608 | 1.06 | 0.736 | 1.52 | 0.079 | 0.84 | 0.299 | 0.98 | 0.897 |
| | | | | | | | (1.01-2.3) | | (0.67-1.27) | | (0.74-1.53) | | (0.95-2.42) | | (0.61-1.17) | | (0.68-1.41) | |
| rs17087049 | A | G | SLC28A3 | Intronic | 0.0993 | D | 1.47 | 0.076 | 1.07 | 0.682 | 1.23 | 0.236 | 1.63 | 0.048 | 1.04 | 0.817 | 1.2 | 0.283 |
| | | | | | | | (0.96-2.25) | | (0.79-1.44) | | (0.87-1.72) | | (1-2.66) | | (0.77-1.4) | | (0.86-1.69) | |
| rs324148 | C | T | SLC29A1 | Intronic | 0.259 | R | 0.51 | 0.043 | 1.09 | 0.639 | 0.89 | 0.666 | 0.62 | 0.174 | 1.25 | 0.252 | 1.06 | 0.819 |
| | | | | | | | (0.27-0.98) | | (0.76-1.58) | | (0.54-1.49) | | (0.31-1.23) | | (0.86-1.81) | | (0.63-1.79) | |
| rs507964 | C | A | SLC29A1 | 850 bp 5′ | 0.349 | R | 1.21 | 0.401 | 1.63 | 0.0009 | 1.21 | 0.326 | 1.5 | 0.12 | 1.88 | 0.00004 | 1.36 | 0.118 |
| | | | | | | | (0.77-1.91) | | (1.22-2.17) | | (0.83-1.77) | | (0.89-2.51) | | (1.39-2.55) | | (0.92-2.01) | |
| rs2396243 | G | A | SLC29A1 | 8.8 kb 5′ | 0.212 | A | 1.18 | 0.525 | 1.67 | 0.008 | 1.21 | 0.412 | 1.24 | 0.477 | 1.72 | 0.008 | 1.19 | 0.461 |
| | | | | | | | (0.701-2.01) | | (1.15-2.43) | | (0.772-1.88) | | (0.688-2.23) | | (1.14-2.58) | | (0.75-1.91) | |

WT = Wild-type;
VAR = variant,
MAF = minor allele frequency;
MOI = Mode of inheritance;
A = additive,
D = dominant,
R = recessive;
MRD1 = Minimal Residual disease after induction 1;
EFS: Event Free Survival;
OS = Overall Survival;
P < 0.05 bold
SNP is associated with detrimental outcome (italic)
SNP is associated with better outcome (underlined)

TABLE 3
Characteristics of enrolled in AML02 trial (discovery cohort) and COG-AAML0531 trial (validation cohort)

| | | AML02 Cohort (N = 166 Patients) | | | COG cohort (N = 931 Patients) | | |
|---|---|---|---|---|---|---|---|
| | | LDAC | HDAC | P- | ADE arm | ADE + GO | |
| Characteristics | Level | (N = 91) | (N = 75) | value | (N = 465) | arm (N 466) | P-value |
| Gender* | Female* | 35⁺ | 38⁺ | 0.156⁺ | 225\hat{ } | 237\hat{ } | 0.491\hat{ } |
| | Male* | 56⁺ | 37⁺ | | 240\hat{ } | 229\hat{ } | |
| Age Group | <10 (Years) | 46 | 37 | 1 | 240 | 233 | 0.669 |
| | >=10 (Years) | 45 | 38 | | 225 | 233 | |
| Age (continuous) | | 8.78(0.013~21.34) | 8.69(0.084~19.28) | 0.441 | 9.08(0.011~29.84) | 9.59(0.022~29.38) | 0.201 |
| Provisional | High* | 25⁺ | 22⁺ | 0.96⁺ | 55\hat{ } | 50\hat{ } | 0.943\hat{ } |
| Risk* | Low* | 28⁺ | 23⁺ | | 168\hat{ } | 166\hat{ } | |
| | Standard* | 38⁺ | 30⁺ | | 232\hat{ } | 240\hat{ } | |
| WBC Group | <30 (G/L) | 45 | 49 | 0.058 | 188 | 185 | 0.908 |
| | >=30 (G/L) | 46 | 26 | | 194 | 190 | |
| WBC | | 61.3(0.061~412.2) | 48.9(0.013~358.1) | 0.486 | 68.99(0.2~526) | 70.56(0.6~827.2) | 0.434 |
| (continuous) | | | | | | | |
| Race* | Black* | 16⁺ | 14⁺ | 0.611⁺ | 54\hat{ } | 50\hat{ } | 0.636\hat{ } |
| | Other* | 9⁺ | 11⁺ | | 28\hat{ } | 21\hat{ } | |
| | Unknown* | 2⁺ | 0⁺ | | 46\hat{ } | 42\hat{ } | |
| | White* | 64⁺ | 50⁺ | | 337\hat{ } | 353\hat{ } | |
| Cytogenetic | 11q23 | 12 | 11 | 0.397 | 54 | 65 | 0.299 |
| Group | inv (16) | 14 | 7 | | 48 | 54 | |
| | Miscell | 21 | 23 | | 151 | 119 | |
| | Normal | 27 | 15 | | 94 | 114 | |
| | t (8; 21) | 10 | 11 | | 67 | 64 | |
| | t (9; 11) | 7 | 6 | | 37 | 35 | |
| MRDIND1* | Negative (<0.1%)* | 49⁺ | 43⁺ | 0.803⁺ | 213\hat{ } | 229\hat{ } | 0.012\hat{ } |
| | Positive (>=0.1%)* | 38⁺ | 28⁺ | | 114\hat{ } | 78\hat{ } | |
| Event-free | 5 years | 0.591 ± 0.0518 | 0.597 ± 0.0569 | 0.898 | 0.467 ± 0.0234 | 0.523 ± 0.0235 | 0.07 |
| Survival | | | | | | | |
| Overall Survival | 5 years | 0.724 ± 0.047 | 0.699 ± 0.0537 | 0.709 | 0.634 ± 0.0228 | 0.659 ± 0.0226 | 0.3 |

TABLE 4
List of 94 SNPs cytarabine metabolism pathway genes that were included in the analysis

| | | | | | MAF | | | |
|---|---|---|---|---|---|---|---|---|
| SNP ID | WT | VAR | Gene | Position | All | Whites | Black | p-value |
| rs580032 | T | G | CDA | Intronic | 0.0903 | 0.057 | 0.1667 | 0.0168 |
| rs639400 | C | T | CDA | 15 kb 5′ | 0.1234 | 0.1316 | 0.1 | 0.8807 |
| rs4655226 | C | T | CDA | Intronic | 0.1385 | 0.1404 | 0.1333 | 0.886 |
| rs818196 | G | C | CDA | Intronic | 0.2228 | 0.1623 | 0.4 | 0.0003 |
| rs12404655 | A | G | CDA | Intronic | 0.2259 | 0.2675 | 0.1167 | 0.0141 |
| rs602950 | T | C | CDA | 5′ UTR | 0.2951 | 0.3465 | 0.0833 | 0.00005 |
| rs1048977 | C | T | CDA | Synonymous | 0.2888 | 0.2636 | 0.3667 | 0.2149 |
| rs10916819 | A | G | CDA | 14 kb 5′ | 0.3915 | 0.3772 | 0.4667 | 0.4633 |
| rs2072671 | A | C | CDA | Missense | 0.2969 | 0.3465 | 0.1 | 0.0006 |
| rs35687416 | C | A | CMPK1 | Missense | 0.067 | 0.0714 | 0.0333 | 0.7645 |
| rs4600090 | C | A | CMPK1 | Intronic | 0.1575 | 0.1667 | 0.069 | 0.0667 |
| rs3088062 | T | C | CMPK1 | 3′ UTR | 0.1939 | 0.2035 | 0.1 | 0.3324 |
| rs17103168 | A | G | CMPK1 | 4 kb 3′ | 0.2168 | 0.2412 | 0.1333 | 0.2194 |
| rs1044457 | C | T | CMPK1 | 3′ UTR | 0.4043 | 0.5 | 0.1167 | 5.70E−07 |
| rs3925058 | T | C | CMPK1 | 1.8 kb 5′ | 0.4427 | 0.5132 | 0.1667 | 2.30E−06 |
| rs7543016 | C | G | CMPK1 | Missense | 0.4276 | 0.5092 | 0.1379 | 2.40E−06 |
| rs7546898 | C | T | CTPS1 | Intronic | 0.3042 | 0.3289 | 0.3333 | 0.461 |
| rs11577910 | G | A | CTPS1 | 3′ UTR | 0.0848 | 0.1053 | 0.0172 | 0.0317 |
| rs4364871 | C | T | CTPS1 | Intronic | 0.1054 | 0.114 | 0.1 | 0.5572 |
| rs4430318 | T | C | CTPS1 | Intronic | 0.1686 | 0.136 | 0.25 | 0.0345 |
| rs12067645 | G | A | CTPS1 | 19 kb 5′ | 0.2409 | 0.2193 | 0.3 | 0.3828 |
| rs12408576 | A | G | CTPS1 | Intronic | 0.3006 | 0.3584 | 0.1207 | 0.0018 |
| rs12729920 | G | A | CTPS1 | 5′ UTR | 0.2957 | 0.323 | 0.3276 | 0.3588 |
| rs7533657 | C | T | CTPS1 | 12 kb 5′ | 0.2875 | 0.2768 | 0.3519 | 0.0802 |
| rs4453027 | G | T | CTPS1 | Intronic | 0.3545 | 0.3628 | 0.3333 | 0.8372 |
| rs4643786 | T | C | DCK | 3′ UTR | 0.1331 | 0.0381 | 0.4821 | 2.49E−09 |
| rs6552622 | C | G | DCTD | Intronic | 0.1024 | 0.0702 | 0.2333 | 0.0039 |
| rs12500335 | A | G | DCTD | 100 kb 5′ | 0.1081 | 0.1045 | 0.1 | 0.2273 |
| rs7278 | C | T | DCTD | 3′ UTR | 0.1363 | 0.1579 | 0.0333 | 0.0586 |
| rs11737887 | T | C | DCTD | 12 kb 5′ | 0.2018 | 0.2237 | 0.1167 | 0.0377 |
| rs6834938 | C | T | DCTD | 429 bp 3′ | 0.2666 | 0.2257 | 0.4167 | 0.0279 |
| rs6829021 | G | A | DCTD | 1.1 kb 3′ | 0.2469 | 0.25 | 0.2833 | 0.8016 |
| rs13147196 | A | G | DCTD | Intronic | 0.2409 | 0.2719 | 0.1167 | 0.0452 |
| rs7277 | G | A | DCTD | 3′ UTR | 0.3484 | 0.3009 | 0.5667 | 0.0031 |
| rs4742 | T | C | DCTD | Synonymous | 0.3323 | 0.3125 | 0.4333 | 0.2627 |
| rs2037067 | G | A | DCTD | 250 kb 5′ | 0.3571 | 0.3682 | 0.3 | 0.6964 |
| rs6850978 | C | T | DCTD | 250 kb 5′ | 0.3848 | 0.4123 | 0.3103 | 0.1766 |
| rs9542 | G | A | DCTD | 3′ UTR | 0.4844 | 0.5273 | 0.2667 | 0.0036 |
| rs3829540 | G | A | NME3 | 17 kb 5′ | 0.1746 | 0.1842 | 0.1833 | 0.6918 |
| rs5841 | C | T | NME4 | Synonymous | 0.4156 | 0.3991 | 0.55 | 0.1341 |
| rs407983 | G | C | NME4 | 4 bp 3′ | 0.4909 | 0.5482 | 0.3667 | 0.0773 |
| rs11191558 | G | A | NT5C2 | Intronic | 0.1111 | 0.1126 | 0.0517 | 0.2839 |
| rs10786736 | G | C | NT5C2 | 3′ UTR | 0.1189 | 0.1239 | 0.0517 | 0.113 |
| rs10883841 | T | C | NT5C2 | Missense | 0.131 | 0.1563 | 0.0667 | 0.2455 |
| rs4917384 | C | T | NT5C2 | 40 kb 3′ | 0.2623 | 0.2883 | 0.0862 | 0.0032 |
| rs1926029 | C | T | NT5C2 | Intronic | 0.3081 | 0.3009 | 0.2833 | 0.769 |
| rs11598702 | T | C | NT5C2 | Intronic | 0.317 | 0.3509 | 0.2414 | 0.1494 |
| rs4917996 | A | C | NT5C2 | Intronic | 0.4393 | 0.4292 | 0.35 | 0.6171 |
| rs1163075 | C | T | NT5C2 | 70 kb 3′ | 0.4596 | 0.4363 | 0.4833 | 0.7643 |
| rs1712517 | A | G | NT5C2 | 80 kb 3′ | 0.4909 | 0.5442 | 0.3667 | 0.0339 |
| rs7802256 | C | T | NT5C3 | 9.5 kb 5′ | 0.0963 | 0.1272 | 0.0167 | 0.0145 |
| rs7776847 | G | A | NT5C3 | Intronic | 0.1867 | 0.1974 | 0.1167 | 0.4078 |
| rs10280692 | C | T | NT5C3 | Intronic | 0.2944 | 0.308 | 0.2167 | 0.1357 |
| rs10252880 | G | C | NT5C3 | 8.2 kb 5′ | 0.3105 | 0.2973 | 0.3036 | 0.6798 |
| rs6946062 | T | C | NT5C3 | Intronic | 0.3855 | 0.4035 | 0.4167 | 0.0835 |
| rs1042927 | A | C | RRM1 | 3′ UTR | 0.1181 | 0.0921 | 0.1897 | 0.0108 |
| rs10767827 | G | T | RRM1 | 13 kb 5′ | 0.1777 | 0.1404 | 0.3 | 0.0171 |
| rs1561876 | T | C | RRM1 | 2 kb 5′ | 0.2101 | 0.1455 | 0.4808 | 1.16E−06 |
| rs12806698 | C | A | RRM1 | 5′ UTR | 0.1951 | 0.1991 | 0.1379 | 0.3772 |
| rs11030918 | T | C | RRM1 | 435 bp 5′ | 0.3048 | 0.3097 | 0.3103 | 0.719 |
| rs183484 | G | T | RRM1 | Synonymous | 0.4478 | 0.5307 | 0.125 | 5.32E−09 |
| rs7110060 | C | G | RRM1 | 9 kb 5′ | 0.4909 | 0.5833 | 0.1667 | 9.87E−09 |
| rs1138729 | A | G | RRM2 | 3′ UTR | 0.1595 | 0.1726 | 0.1897 | 0.5461 |
| rs6759180 | A | G | RRM2 | Intronic | 0.2687 | 0.3165 | 0.0833 | 0.0033 |
| rs1130609 | G | T | RRM2 | Missense | 0.2763 | 0.3091 | 0.0862 | 0.0117 |
| rs6742272 | C | G | RRM2 | 18 kb 5′ | 0.3253 | 0.3772 | 0.2333 | 0.1704 |
| rs8187763 | C | T | SLC28A1 | Intronic | 0.1024 | 0.1228 | 0.0667 | 0.4602 |
| rs4980345 | G | A | SLC28A1 | Intronic | 0.1265 | 0.0965 | 0.1667 | 0.0509 |
| rs2122859 | C | A | SLC28A1 | Intronic | 0.2018 | 0.2105 | 0.1 | 0.1484 |
| rs3743162 | C | T | SLC28A1 | Intronic | 0.1878 | 0.2168 | 0.1167 | 0.3983 |
| rs11853372 | G | T | SLC28A1 | Intronic | 0.3283 | 0.364 | 0.3167 | 0.1949 |
| rs4980347 | C | T | SLC28A1 | Intronic | 0.4 | 0.385 | 0.45 | 0.6789 |
| rs4247411 | C | T | SLC28A1 | 3.3 kb 3′ | 0.4277 | 0.4693 | 0.35 | 0.2249 |
| rs4271567 | T | A | SLC28A1 | Intronic | 0.4787 | 0.4342 | 0.569 | 0.0461 |
| rs11140521 | T | C | SLC28A3 | Intronic | 0.0662 | 0.0132 | 0.3167 | 4.39E−09 |
| rs17087049 | A | G | SLC28A3 | Intronic | 0.0993 | 0.0526 | 0.2667 | 7.58E−05 |
| rs12554292 | C | T | SLC28A3 | 83 kb 5′ | 0.0975 | 0.0885 | 0.15 | 0.4028 |
| rs4877839 | G | A | SLC28A3 | Intronic | 0.108 | 0.0982 | 0.1429 | 0.2665 |
| rs10868154 | T | A | SLC28A3 | 1.6 kb 5′ | 0.1242 | 0.146 | 0.05 | 0.3496 |
| rs7874422 | C | T | SLC28A3 | Intronic | 0.1325 | 0.1184 | 0.2 | 0.208 |
| rs7874528 | G | T | SLC28A3 | 60 kb 3′ | 0.1484 | 0.1228 | 0.2931 | 0.0062 |
| rs885003 | T | C | SLC28A3 | Intronic | 0.1927 | 0.1316 | 0.4333 | 1.03E−05 |
| rs7029691 | G | A | SLC28A3 | Intronic | 0.3132 | 0.2149 | 0.7667 | 2.03E−12 |
| rs4877831 | C | G | SLC28A3 | Intronic | 0.3484 | 0.2699 | 0.65 | 6.90E−06 |
| rs4877842 | A | G | SLC28A3 | Intronic | 0.3212 | 0.2699 | 0.4833 | 0.0139 |
| rs11140525 | G | A | SLC28A3 | Intronic | 0.3554 | 0.2895 | 0.6167 | 8.27E−06 |
| rs7867504 | T | C | SLC28A3 | Synonymous | 0.4393 | 0.3451 | 0.8 | 6.31E−08 |
| rs17343066 | G | A | SLC28A3 | Intronic | 0.4598 | 0.5357 | 0.1552 | 1.27E−06 |
| rs747199 | C | G | SLC29A1 | Intronic | 0.1708 | 0.1955 | 0.0833 | 0.2247 |
| rs9472236 | G | A | SLC29A1 | 4.9 kb 3′ | 0.1787 | 0.1842 | 0.0667 | 0.0204 |
| rs2396243 | G | A | SLC29A1 | 8.8 kb 5′ | 0.2125 | 0.2027 | 0.2069 | 0.8397 |
| rs324148 | C | T | SLC29A1 | Intronic | 0.259 | 0.2895 | 0.15 | 0.1648 |
| rs507964 | C | A | SLC29A1 | 850 bp 5′ | 0.3493 | 0.3596 | 0.25 | 0.2019 |
| rs760370 | A | G | SLC29A1 | Intronic | 0.3192 | 0.3158 | 0.3833 | 0.3122 |

TABLE 5
Genetic variants associated with clinical outcomes within LDAC and HDAC arms of AML02 cohort

| | | | | | Low dose arm (LDAC; 91 Patients) | | | | | | High dose arm (HDAC; 75 Patients) | | | | | |
|---|---|---|---|---|---|---|---|---|---|---|---|---|---|---|---|---|
| | | | | | MRD1 | | EFS | | OS | | MRD1 | | EFS | | OS | |
| | | | | | | P | | P | | P | | P | | P | | P |
| SNP | WT | VAR | Gene | MOI | OR (95% CI) | value | HR (95% CI) | value | HR (95% CI) | value | OR (95% CI) | value | HR (95% CI) | value | HR (95% CI) | value |
| rs10916819 | A | G | CDA | D | 1.75 (1.08-2.83) | 0.023 | 1.77 (1.14-2.75) | 0.0103 | 2.05 (1.12-3.74) | 0.019 | 1.87 (1.09-3.21) | 0.023 | 1.08 (0.75-1.56) | 0.693 | 1.33 (0.83-2.12) | 0.235 |
| rs1048977 | C | T | CDA | D | 1.21 (0.79-1.85) | 0.385 | 1.48 (1.06-2.08) | 0.022 | 1.14 (0.77-1.69) | 0.519 | 1.09 (0.67-1.78) | 0.726 | 0.9 (0.63-1.28) | 0.546 | 1.44 (0.7-2.98) | 0.326 |
| rs2072671 | A | C | CDA | A | 0.57 (0.31-1.04) | 0.067 | 0.58 (0.35-0.95) | 0.032 | 0.68 (0.38-1.23) | 0.201 | 0.73 (0.34-1.56) | 0.414 | 1.18 (0.7-1.99) | 0.532 | 0.89 (0.46-1.75) | 0.744 |
| rs602950 | T | C | CDA | A | 0.57 (0.3-1.08) | 0.083 | 0.56 (0.33-0.96) | 0.034 | 0.68 (0.36-1.26) | 0.219 | 0.69 (0.32-1.48) | 0.334 | 1.25 (0.74-2.09) | 0.401 | 0.86 (0.44-1.69) | 0.657 |
| rs580032 | T | G | CDA | D | 1.16 (0.63-2.13) | 0.635 | 0.86 (0.51-1.44) | 0.555 | 1.12 (0.65-1.9) | 0.689 | 0.91 (0.49-1.66) | 0.751 | 0.45 (0.22-0.92) | 0.029 | 0.58 (0.28-1.19) | 0.137 |
| rs818196 | G | C | CDA | D | 1.28 (0.82-2) | 0.268 | 1.18 (0.85-1.64) | 0.325 | 1.09 (0.73-1.62) | 0.677 | 0.9 (0.56-1.47) | 0.683 | 0.66 (0.45-0.98) | 0.037 | 0.73 (0.47-1.14) | 0.169 |
| rs17103168 | A | G | CMPK1 | D | 0.55 (0.35-0.87) | 0.009 | 0.96 (0.69-1.32) | 0.787 | 1.03 (0.69-1.52) | 0.898 | 0.62 (0.37-1.04) | 0.072 | 1.05 (0.73-1.51) | 0.796 | 0.9 (0.58-1.41) | 0.652 |
| rs7543016 | C | G | CMPK1 | R | 2.01 (1.13-3.58) | 0.018 | 0.85 (0.55-1.32) | 0.479 | 0.67 (0.37-1.23) | 0.201 | 1.29 (0.69-2.42) | 0.425 | 0.69 (0.38-1.25) | 0.219 | 0.65 (0.32-1.35) | 0.25 |
| rs3925058 | T | C | CMPK1 | D | 0.6 (0.38-0.95) | 0.029 | 0.7 (0.51-0.97) | 0.033 | 0.76 (0.51-1.13) | 0.176 | 1.41 (0.79-2.53) | 0.246 | 0.81 (0.56-1.18) | 0.275 | 0.76 (0.49-1.18) | 0.219 |
| rs3088062 | T | C | CMPK1 | A | 2.37 (1.06-5.28) | 0.035 | 1.66 (0.99-2.8) | 0.055 | 1.63 (0.89-3.01) | 0.116 | 1.16 (0.48-2.81) | 0.736 | 1.41 (0.8-2.51) | 0.236 | 1.48 (0.75-2.94) | 0.259 |
| rs7533657 | C | T | CTPS1 | R | 0.9 (0.36-2.27) | 0.8275 | 1.12 (0.62-2.02) | 0.708 | 1.05 (0.51-2.16) | 0.893 | 1.31 (0.67-2.57) | 0.434 | 1.76 (1.14-2.7) | 0.009 | 1.97 (1.22-3.18) | 0.005 |
| rs4643786 | T | C | DCK | R | 2.34 (0.76-7.15) | 0.137 | 2.15 (1.28-3.64) | 0.004 | 1.7 (0.92-3.11) | 0.088 | 0.87 (0.36-2.12) | 0.763 | 1.15 (0.63-2.09) | 0.658 | 1.38 (0.75-2.56) | 0.303 |
| rs4742 | T | C | DCTD | R | 1.19 (0.57-2.46) | 0.646 | 1.49 (0.93-2.4) | 0.0962 | 1.96 (1.2-3.21) | 0.007 | 1.08 (0.49-2.38) | 0.845 | 1.53 (0.98-2.4) | 0.061 | 1.49 (0.91-2.46) | 0.116 |
| rs6834938 | C | T | DCTD | R | 1.92 (0.82-4.48) | 0.134 | 1.4 (0.83-2.35) | 0.209 | 1.75 (1.02-3) | 0.043 | 1.08 (0.49-2.38) | 0.845 | 0.7 (0.34-1.44) | 0.332 | 0.86 (0.42-1.79) | 0.695 |
| rs11737887 | T | C | DCTD | A | 0.95 (0.4-2.25) | 0.899 | 1.39 (0.73-2.66) | 0.318 | 1.76 (0.8-3.86) | 0.158 | 2.74 (1.06-7.06) | 0.037 | 0.96 (0.49-1.89) | 0.916 | 0.95 (0.43-2.12) | 0.903 |
| rs6552622 | C | G | DCTD | D | 0.86 (0.49-1.49) | 0.582 | 1.05 (0.7-1.59) | 0.799 | 1.22 (0.77-1.94) | 0.389 | 1.06 (0.56-1.99) | 0.862 | 1.71 (1.15-2.53) | 0.007 | 1.84 (1.19-2.85) | 0.006 |
| rs3829540 | G | A | NME3 | D | 1.15 (0.73-1.8) | 0.541 | 1.23 (0.89-1.71) | 0.211 | 1.22 (0.82-1.82) | 0.328 | 0.82 (0.49-1.37) | 0.453 | 0.71 (0.47-1.09) | 0.118 | 0.51 (0.27-0.93) | 0.028 |
| rs407983 | G | C | NME4 | A | 0.92 (0.53-1.59) | 0.768 | 1.36 (0.88-2.09) | 0.166 | 1.73 (1-2.98) | 0.049 | 1.43 (0.76-2.67) | 0.264 | 1.1 (0.7-1.72) | 0.674 | 1.05 (0.62-1.8) | 0.845 |
| rs5841 | C | T | NME4 | D | 0.7 (0.45-1.08) | 0.104 | 0.94 (0.68-1.31) | 0.734 | 0.86 (0.58-1.27) | 0.446 | 0.48 (0.28-0.81) | 0.006 | 0.86 (0.6-1.23) | 0.413 | 1.15 (0.74-1.81) | 0.536 |
| rs11191558 | G | A | NT5C2 | D | 0.96 (0.56-1.64) | 0.875 | 1.49 (1.05-2.1) | 0.027 | 1.32 (0.85-2.05) | 0.216 | 0.93 (0.53-1.66) | 0.816 | 0.87 (0.56-1.37) | 0.56 | 0.84 (0.48-1.44) | 0.522 |
| rs10786736 | G | C | NT5C2 | D | 1.1 (0.66-1.83) | 0.714 | 1.45 (1.03-2.03) | 0.033 | 1.37 (0.9-2.08) | 0.145 | 0.9 (0.51-1.59) | 0.709 | 0.92 (0.6-1.4) | 0.697 | 0.92 (0.56-1.52) | 0.748 |
| rs4917384 | C | T | NT5C2 | D | 0.78 (0.51-1.19) | 0.252 | 1.04 (0.76-1.44) | 0.794 | 0.87 (0.58-1.29) | 0.476 | 0.73 (0.44-1.2) | 0.215 | 0.74 (0.51-1.08) | 0.118 | 0.61 (0.38-0.98) | 0.039 |
| rs6946062 | T | C | NT5C3 | D | 0.94 (0.61-1.46) | 0.794 | 0.75 (0.54-1.04) | 0.083 | 0.58 (0.39-0.87) | 0.008 | 0.96 (0.59-1.56) | 0.86 | 1.13 (0.78-1.63) | 0.5244 | 1.47 (0.92-2.35) | 0.108 |
| rs10252880 | G | C | NT5C3 | A | 0.97 (0.51-1.82) | 0.915 | 1.21 (0.74-1.98) | 0.435 | 1.98 (1.13-3.44) | 0.016 | 1.18 (0.62-2.27) | 0.61 | 0.99 (0.62-1.57) | 0.958 | 0.74 (0.42-1.32) | 0.307 |
| rs7776847 | G | A | NT5C3 | A | 0.92 (0.42-2) | 0.831 | 1.15 (0.64-2.09) | 0.637 | 1.91 (1.01-3.62) | 0.046 | 1.23 (0.57-2.65) | 0.593 | 0.83 (0.46-1.5) | 0.538 | 0.71 (0.34-1.47) | 0.352 |
| rs10767827 | G | T | RRM1 | D | 0.98 (0.6-1.61) | 0.931 | 1.09 (0.76-1.57) | 0.643 | 0.97 (0.61-1.53) | 0.887 | 0.55 (0.31-0.97) | 0.039 | 1.03 (0.71-1.49) | 0.875 | 1.04 (0.68-1.61) | 0.85 |
| rs12806698 | C | A | RRM1 | D | 1.14 (0.72-1.8) | 0.573 | 0.98 (0.69-1.39) | 0.901 | 1.19 (0.79-1.79) | 0.401 | 0.89 (0.54-1.45) | 0.631 | 0.64 (0.43-0.96) | 0.033 | 0.62 (0.37-1.02) | 0.059 |
| rs6742272 | C | G | RRM2 | A | 0.49 (0.25-0.96) | 0.038 | 1.5 (0.95-2.36) | 0.08 | 1.43 (0.83-2.48) | 0.202 | 0.86 (0.44-1.68) | 0.663 | 1.09 (0.68-1.76) | 0.718 | 1.09 (0.62-1.92) | 0.762 |
| rs1138729 | A | G | RRM2 | D | 1.19 (0.73-1.94) | 0.478 | 1.55 (1.11-2.18) | 0.011 | 1.41 (0.93-2.14) | 0.11 | 1.04 (0.61-1.77) | 0.877 | 1.14 (0.78-1.66) | 0.495 | 1.42 (0.93-2.17) | 0.108 |
| rs4980345 | G | A | SLC28A1 | A | 1.08 (0.45-2.58) | 0.862 | 1.81 (1-3.26) | 0.049 | 1.84 (0.95-3.55) | 0.07 | 0.55 (0.19-1.62) | 0.282 | 0.94 (0.47-1.87) | 0.857 | 1.3 (0.64-2.64) | 0.461 |
| rs3743162 | C | T | SLC28A1 | D | 0.77 (0.48-1.23) | 0.274 | 0.82 (0.56-1.19) | 0.298 | 1.07 (0.71-1.64) | 0.739 | 1.89 (1.15-3.11) | 0.013 | 1.65 (1.15-2.36) | 0.007 | 1.43 (0.94-2.18) | 0.093 |
| rs4247411 | C | T | SLC28A1 | D | 0.94 (0.59-1.51) | 0.802 | 1.05 (0.73-1.51) | 0.781 | 1.03 (0.67-1.59) | 0.892 | 1.91 (1.08-3.37) | 0.026 | 1.69 (1.08-2.64) | 0.022 | 1.72 (1-2.95) | 0.051 |
| rs4271567 | T | A | SLC28A1 | R | 0.76 (0.46-1.28) | 0.305 | 1 (0.69-1.46) | 0.997 | 1.12 (0.72-1.74) | 0.612 | 0.66 (0.35-1.23) | 0.187 | 0.52 (0.29-0.95) | 0.032 | 0.54 (0.26-1.11) | 0.094 |
| rs7874528 | G | T | SLC28A3 | D | 1.93 (1.12-3.32) | 0.017 | 0.97 (0.66-1.44) | 0.893 | 0.89 (0.54-1.45) | 0.637 | 0.91 (0.52-1.61) | 0.749 | 1.24 (0.85-1.81) | 0.257 | 1.41 (0.91-2.17) | 0.125 |
| rs11140525 | G | A | SLC28A3 | A | 1.44 (0.81-2.58) | 0.216 | 0.98 (0.63-1.54) | 0.935 | 1.16 (0.68-1.97) | 0.589 | 0.44 (0.21-0.94) | 0.033 | 0.73 (0.44-1.21) | 0.226 | 0.87 (0.49-1.56) | 0.645 |
| rs12554292 | C | T | SLC28A3 | D | 1.26 (0.72-2.21) | 0.409 | 0.71 (0.42-1.2) | 0.2 | 0.78 (0.43-1.42) | 0.418 | 1.92 (1.04-3.56) | 0.037 | 1.15 (0.75-1.76) | 0.514 | 1.4 (0.87-2.24) | 0.168 |
| rs324148 | C | T | SLC29A1 | A | 0.37 (0.17-0.78) | 0.009 | 1.35 (0.85-2.13) | 0.202 | 1.14 (0.64-2.04) | 0.653 | 1.08 (0.55-2.1) | 0.832 | 0.88 (0.52-1.5) | 0.644 | 0.75 (0.39-1.46) | 0.401 |
| rs2396243 | G | A | SLC29A1 | D | 0.92 (0.59-1.43) | 0.697 | 1.56 (1.12-2.16) | 0.008 | 1.59 (1.07-2.36) | 0.021 | 1.39 (0.84-2.31) | 0.203 | 0.94 (0.63-1.39) | 0.752 | 0.87 (0.54-1.4) | 0.573 |
| rs507964 | C | A | SLC29A1 | R | 1.41 (0.76-2.62) | 0.276 | 1.54 (1.04-2.29) | 0.03 | 1.1 (0.64-1.88) | 0.731 | 1.01 (0.51-2.01) | 0.969 | 1.71 (1.12-2.62) | 0.013 | 1.37 (0.8-2.36) | 0.255 |
| rs747199 | C | G | SLC29A1 | D | 1.18 (0.75-1.88) | 0.475 | 0.88 (0.61-1.28) | 0.509 | 0.55 (0.3-1) | 0.049 | 1.37 (0.82-2.29) | 0.233 | 0.97 (0.66-1.42) | 0.879 | 1.1 (0.72-1.71) | 0.653 |

WT = Wild-type;
VAR = variant,
MAF = minor allele frequency;
MOI = Mode of inheritance;
A = additive,
D = dominant,
R = recessive;
MRD1 = Minimal Residual disease after induction 1;
EFS: Event Free Survival;
OS = Overall Survival;
P < 0.05 bold
SNP is associated with detrimental outcome (italic)
SNP is associated with better outcome (underlined)

TABLE 6A
SNP combination models with lowest BIC for
association with MRD1 and 1000 permutations

| SNP1 | SNP2 | SNP3 | BIC | wgt | P. perm(1000) |
|---|---|---|---|---|---|
| rs10916819.dom | rs5841.dom | rs7543016.rec | 199.84 | 0.121 | 0.084 |
| rs10916819.dom | rs5841.dom | rs1044457.rec | 201.23 | 0.061 | 0.098 |
| rs10916819.dom | rs5841.dom | rs17103168.dom | 201.37 | 0.056 | 0.047 |
| rs10916819.dom | rs5841.dom | rs1048977.add | 202.06 | 0.040 | 0.064 |
| rs10916819.dom | rs5841.dom | rs2037067.rec | 203.35 | 0.021 | 0.271 |

TABLE 6B
Multivariable analysis of top model SNPs with 1000
perm p-value < 0.05 for association with MRD1

| SNPid | Gene | OR | OR95LB | OR95UB | P value |
|---|---|---|---|---|---|
| rs10916819.dom | CDA | 1.974 | 1.34 | 2.99 | 0.0008 |
| rs17103168.dom | CMPK1 | 0.574 | 0.392 | 0.826 | 0.003 |
| rs5841.dom | NME4 | 0.492 | 0.331 | 0.714 | 0.00028 |

TABLE 7A
SNP combination models with lowest BIC for
association with EFS and 1000 permutations

| SNP1 | SNP2 | SNP3 | BIC | wgt | P. perm (1000) |
|---|---|---|---|---|---|
| rs2396243.add | rs1044457.dom | rs1138729.dom | 570.428 | 0.946 | 0.059 |
| rs2396243.add | rs1044457.dom | rs7543016.add | 577.818 | 0.023 | 0.895 |
| rs2396243.add | rs1044457.dom | rs2037067.add | 578.803 | 0.014 | 0.791 |
| rs2396243.add | rs1044457.dom | rs6759180.rec | 580.076 | 0.008 | 0.775 |
| rs2396243.add | rs1044457.dom | rs1163075.add | 582.507 | 0.002 | 0.906 |

TABLE 7B
Multivariable analysis of top model SNPs with lowest
1000 perm p-value for association with EFS

| SNPid | Gene | HR | HR95LB | HR95UB | P value |
|---|---|---|---|---|---|
| rs2396243.add | SLC29A1 | 1.91 | 1.29 | 2.83 | 0.0013 |
| rs1044457.dom | CMPK1 | 0.718 | 0.554 | 0.932 | 0.0127 |
| rs1138729.dom | RRM2 | 1.32 | 1.01 | 1.72 | 0.0417 |

TABLE 8
SNPs that are included to be part of Composite ACS10 SNP Score = Σ (genotype score SNP 1 + SNP 2 . . . + SNP10)

| SNP | Gene | Position | Ancestral | Variant | End point | Direction | MOI | Genotype score |
|---|---|---|---|---|---|---|---|---|
| rs10916819 | CDA | 14 kb 5′ | A | G | MRD1 | Detrimental | Dominant | AA = 0, AG/GG = −1 |
| rs17103168 | CMPK1 | 4 kb 3′ | A | G | MRD1 | Beneficial | Dominant | AA = 0, AG/GG = 1 |
| rs5841 | NME4 | Syn | C | T | MRD1 | Beneficial | Dominant | CC = 0, CT/TT = 1 |
| rs2396243 | SLC29A1 | 8.8 kb 5′ | G | A | EFS | Detrimental | Additive | GG = 0, AG = −1, AA = −2 |
| rs1044457 | CMPK1 | 3′ UTR | C | T | EFS | Beneficial | Dominant | CC = 0, CT/TT = 1 |
| rs1138729 | RRM2 | 3′ UTR | A | G | EFS | Detrimental | Dominant | AA = 0, AG/GG = − 1 |
| rs4643786 | DCK | 3′ UTR | T | C | Ara-CTP | Detrimental | Additive | TT = 0, CT = −1, CC = −2 |
| rs11030918 | RRM1 | 435 bp 5′ | T | C | Ara-CTP | Beneficial | Recessive | TT/CT = 0, CC = 1 |
| rs12067645 | CTPS1 | 19 kb 5′ | G | A | Ara-CTP | Beneficial | Additive | GG = 0, AG = 1, AA = 2 |
| rs17343066 | SLC28A3 | Intronic | G | A | Ara-CTP | Beneficial | Recessive | GG/AG = 0, AA = 1 |

TABLE 9
Characteristics of 166 patients enrolled in AML02 cohort and within low and high dose arms within AML02 cohort by ACS10 score groups

| | | AML02 Whole Cohort | | | AML02 Low dose ara-C (LDAC arm; | | |
|---|---|---|---|---|---|---|---|
| | | (N = 166 Patients) | | | N = 91 Patients) | | |
| | | High ACS10 | Low ACS10 | | High ACS10 | Low ACS10 | |
| | | score | score | | score | score | |
| | | group | group | P- | group | group | P- |
| Characteristics | Level | (N = 97) | (N = 69) | value | (N = 53) | (N = 38) | value |
| Gender | Female | 43 | 30 | 0.99 | 22 | 13 | 0.626 |
| | Male | 54 | 39 | | 31 | 25 | |
| Age Group | <10(Years) | 44 | 39 | 0.208 | 24 | 22 | 0.33 |
| | >=10(Years) | 53 | 30 | | 29 | 16 | |
| Age (continuous) | | 9.18(0.014~21.35) | 8.13(0.085~20.11) | 0.298 | 9.31(0.014~21.35) | 8.04(0.493~20.11) | 0.346 |
| Provisional Risk | High | 24 | 23 | 0.478 | 12 | 13 | 0.466 |
| | Low | 31 | 20 | | 17 | 11 | |
| | Standard | 42 | 26 | | 24 | 14 | |
| WBC Group | <30(G/L) | 54 | 40 | 0.892 | 24 | 21 | 0.468 |
| | >=30(G/L) | 43 | 29 | | 29 | 17 | |
| WBC (continuous) | | 57.2(0.028~412.2) | 53.7(0.013~−358.1) | 0.931 | 70.59(0.061~412.2) | 48.44(1~291.7) | 0.409 |
| Race | Black | 8 | 22 | 0.0002 | 5 | 11 | 0.064 |
| | Other | 12 | 8 | | 5 | 4 | |
| | Unknown | 1 | 1 | | 1 | 1 | |
| | White | 76 | 38 | | 42 | 22 | |
| Cytogenetic Group | 11q23 | 17 | 6 | 0.144 | 8 | 4 | 0.896 |
| | inv (16) | 15 | 6 | | 9 | 5 | |
| | Miscell | 19 | 25 | | 11 | 10 | |
| | Normal | 25 | 17 | | 14 | 13 | |
| | t (8; 21) | 11 | 10 | | 6 | 4 | |
| | t (9; 11) | 8 | 5 | | 5 | 2 | |
| Event-free Survival | 5 years | 0.675 ± 0.048 | 0.478 ± 0.06 | 0.005 | 0.712 ± 0.063 | 0.421 ± 0.08 | 0.002 |
| Overall Survival | 5 years | 0.79 ± 0.042 | 0.603 ± 0.059 | 0.008 | 0.829 ± 0.052 | 0.579 ± 0.08 | 0.009 |

| | | AML02 High dose ara-C (HDAC arm; N = 75 Patients) | | |
|---|---|---|---|---|
| | | High ACS10 score | Low ACS10 score | |
| Characteristics | Level | group (N = 44) | group (N = 31) | P-value |
| Gender | Female | 21 | 17 | 0.709 |
| | Male | 23 | 14 | |
| Age Group | <10(Years) | 20 | 17 | 0.571 |
| | >=10(Years) | 24 | 14 | |
| Age (continuous) | | 9.02(0.014~21.35) | 8.23(0.493~20.11) | 0.591 |
| Provisional Risk | High | 12 | 10 | 0.895 |
| | Low | 14 | 9 | |
| | Standard | 18 | 12 | |
| WBC Group | <30(G/L) | 30 | 19 | 0.711 |
| | >=30(G/L) | 14 | 12 | |
| WBC (continuous) | | 41.06(0.028~351) | 60.15(0.013~358.1) | 0.375 |
| Race | Black | 3 | 11 | 0.009 |
| | Other | 7 | 4 | |
| | Unknown | 0 | 0 | |
| | White | 34 | 16 | |
| Cytogenetic Group | 11q23 | 9 | 2 | 0.0317 |
| | inv (16) | 6 | 1 | |
| | Miscell | 8 | 15 | |
| | Normal | 11 | 4 | |
| | t (8; 21) | 5 | 6 | |
| | t (9; 11) | 3 | 3 | |
| Event-free Survival | 5 years | 0.631 ± 0.074 | 0.548 ± 0.089 | 0.49 |
| Overall Survival | 5 years | 0.743 ± 0.067 | 0.634 ± 0.088 | 0.298 |

TABLE 10
Characteristics of 931 patients from COG validation dataset by ACS10 score groups and treatment arm

| | | COG-ADE arm (N = 465 Patients) | | | COG-ADE + GO arm (N = 466 Patients) | | |
|---|---|---|---|---|---|---|---|
| | | High ACS10 | Low ACS10 | | High ACS10 | Low ACS10 | |
| | | score | score | | score | score | |
| | | group | group | | group | group | |
| Characteristics | Level | (N = 316) | (N = 149) | P-value | (N = 284) | (N = 182) | P-value |
| Gender | Female | 148 | 77 | 0.381 | 137 | 100 | 0.188 |
| | Male | 168 | 72 | | 147 | 82 | |
| Age Group | <10(Years) | 159 | 81 | 0.475 | 143 | 90 | 0.924 |
| | >=10(Years) | 157 | 68 | | 141 | 92 | |
| Age (continuous) | | 9.14(0.011~23.94) | 8.94(0.027~29.84) | 0.673 | 9.65(0.022~23.8) | 9.51(0.025~29.37) | 0.649 |
| Provisional Risk | High | 39 | 16 | 0.321 | 37 | 13 | 0.157 |
| | Low | 119 | 49 | | 101 | 65 | |
| | Standard | 149 | 83 | | 143 | 97 | |
| WBC Group | <30(G/L) | 127 | 61 | 0.997 | 122 | 73 | 0.841 |
| | >=30(G/L) | 130 | 64 | | 116 | 74 | |
| WBC (continuous) | | 63.92(1.1~447.3) | 79.4(0.2~526) | 0.91 | 72.76(0.8~827.2) | 67.01(0.6~415.7) | 0.754 |
| Race | Black | 19 | 35 | 3.46E−09 | 14 | 28 | 9.41E−06 |
| | Other | 13 | 15 | | 7 | 11 | |
| | Unknown | 29 | 17 | | 19 | 18 | |
| | White | 255 | 82 | | 198 | 90 | |
| Cytogenetic Group | 11q23 | 34 | 20 | 0.945 | 32 | 23 | 0.089 |
| | inv (16) | 35 | 13 | | 32 | 18 | |
| | Miscell | 101 | 50 | | 64 | 27 | |
| | Normal | 63 | 31 | | 55 | 43 | |
| | t (8; 21) | 48 | 19 | | 30 | 25 | |
| | t (9; 11) | 25 | 12 | | 19 | 5 | |
| Event-free Survival | 5 years | 0.493 ± 0.0.029 | 0.409 ± 0.041 | 0.026 | 0.539 ± 0.03 | 0.501 ± 0.038 | 0.67 |
| Overall Survival | 5 years | 0.683 ± 0.027 | 0.529 ± 0.0267 | 0.0016 | 0.678 ± 0.028 | 0.628 ± 0.0372 | 0.758 |

TABLE 11
Interaction of arm with ACS10 score as a continuous variable in terms of EFS/OS outcomes at
a fixed time in a risk-adjusted Cox model in LDAC and HDAC treatment arms of AML02 cohort

| | 3-year EFS | | 4-year EFS | | 5-year EFS | | 3-year OS | | 4-year OS | | 5-year OS | |
|---|---|---|---|---|---|---|---|---|---|---|---|---|
| Score | LDAC | HDAC | LDAC | HDAC | LDAC | HDAC | LDAC | HDAC | LDAC | HDAC | LDAC | HDAC |
| −5 | 0.0743 | 0.5123 | 0.0683 | 0.4983 | 0.0652 | 0.4906 | 0.3280 | 0.5073 | 0.3197 | 0.4983 | 0.3197 | 0.4983 |
| −3 | 0.2202 | 0.5513 | 0.2083 | 0.5377 | 0.2020 | 0.5302 | 0.4846 | 0.5873 | 0.4756 | 0.5788 | 0.4756 | 0.5788 |
| −2 | 0.3251 | 0.5703 | 0.3112 | 0.5569 | 0.3037 | 0.5496 | 0.5632 | 0.6251 | 0.5546 | 0.6170 | 0.5546 | 0.6170 |
| −1 | 0.4399 | 0.5889 | 0.4255 | 0.5758 | 0.4176 | 0.5686 | 0.6369 | 0.6609 | 0.6289 | 0.6533 | 0.6289 | 0.6533 |
| 0 | 0.5527 | 0.6070 | 0.5391 | 0.5942 | 0.5316 | 0.5872 | 0.7030 | 0.6946 | 0.6960 | 0.6875 | 0.6960 | 0.6875 |
| 1 | 0.6541 | 0.6247 | 0.6423 | 0.6122 | 0.6357 | 0.6053 | 0.7604 | 0.7260 | 0.7544 | 0.7194 | 0.7544 | 0.7194 |
| 2 | 0.7393 | 0.6418 | 0.7296 | 0.6297 | 0.7242 | 0.6231 | 0.8089 | 0.7550 | 0.8039 | 0.7489 | 0.8039 | 0.7489 |
| 3 | 0.8073 | 0.6585 | 0.7997 | 0.6468 | 0.7955 | 0.6403 | 0.8489 | 0.7816 | 0.8448 | 0.7760 | 0.8448 | 0.7760 |
| 4 | 0.8596 | 0.6747 | 0.8539 | 0.6633 | 0.8507 | 0.6570 | 0.8814 | 0.8058 | 0.8781 | 0.8007 | 0.8781 | 0.8007 |
| 5 | 0.8988 | 0.6903 | 0.8946 | 0.6793 | 0.8922 | 0.6732 | 0.9074 | 0.8278 | 0.9048 | 0.8232 | 0.9048 | 0.8232 |

TABLE 12
Interaction of arm with ACS10 score as a continuous variable in terms of EFS/OS outcomes at
a fixed time in a risk-adjusted Cox model in ADE and ADE + GO treatment arms of AAML0531

| | 3-year EFS | | 4-year EFS | | 5-year EFS | | 3-year OS | | 4-year OS | | 5-year OS | |
|---|---|---|---|---|---|---|---|---|---|---|---|---|
| Score | ADE | ADE + GO | ADE | ADE + GO | ADE | ADE + GO | ADE | ADE + GO | ADE | ADE + GO | ADE | ADE + GO |
| −5 | 0.2529 | 0.5334 | 0.2438 | 0.5238 | 0.2404 | 0.5201 | 0.3737 | 0.7050 | 0.3469 | 0.6847 | 0.3264 | 0.6684 |
| −4 | 0.2874 | 0.5347 | 0.2780 | 0.5251 | 0.2743 | 0.5214 | 0.4258 | 0.7056 | 0.3986 | 0.6853 | 0.3776 | 0.6690 |
| −3 | 0.3234 | 0.5360 | 0.3136 | 0.5265 | 0.3098 | 0.5227 | 0.4778 | 0.7061 | 0.4508 | 0.6859 | 0.4298 | 0.6696 |
| −2 | 0.3603 | 0.5373 | 0.3503 | 0.5278 | 0.3464 | 0.5241 | 0.5287 | 0.7067 | 0.5024 | 0.6865 | 0.4817 | 0.6702 |
| −1 | 0.3978 | 0.5386 | 0.3876 | 0.5291 | 0.3838 | 0.5254 | 0.5776 | 0.7073 | 0.5524 | 0.6871 | 0.5325 | 0.6708 |
| 0 | 0.4354 | 0.5399 | 0.4252 | 0.5304 | 0.4213 | 0.5267 | 0.6238 | 0.7078 | 0.6001 | 0.6877 | 0.5812 | 0.6715 |
| 1 | 0.4727 | 0.5412 | 0.4627 | 0.5317 | 0.4588 | 0.5280 | 0.6668 | 0.7084 | 0.6448 | 0.6883 | 0.6272 | 0.6721 |
| 2 | 0.5093 | 0.5425 | 0.4995 | 0.5330 | 0.4957 | 0.5293 | 0.7064 | 0.7089 | 0.6862 | 0.6889 | 0.6700 | 0.6727 |
| 3 | 0.5450 | 0.5438 | 0.5355 | 0.5343 | 0.5318 | 0.5306 | 0.7425 | 0.7095 | 0.7242 | 0.6895 | 0.7093 | 0.6733 |
| 4 | 0.5795 | 0.5451 | 0.5703 | 0.5356 | 0.5668 | 0.5319 | 0.7750 | 0.7101 | 0.7585 | 0.6900 | 0.7451 | 0.6739 |
| 5 | 0.6125 | 0.5464 | 0.6038 | 0.5369 | 0.6004 | 0.5332 | 0.8041 | 0.7106 | 0.7893 | 0.6906 | 0.7773 | 0.6745 |

1. A method for characterizing a subject having cancer, the method comprising:
(i) performing an assay to identify presence of nucleotides of nucleic acids in a biological sample obtained from a subject;
(ii) assigning a genotype score based upon the nucleotides present at each of a set of single-nucleotide polymorphism (SNP) locations comprising rs10916819, rs17103168, rs5841, rs2396243, rs1044457, rs1138729, rs4643786, rs11030918, rs12067645, and rs17343066 in the biological sample, said genotype score being assigned according to a method comprising:
(a) assigning a genotype score for the SNP location rs10916819 of zero (0) when the characterized nucleotides are AA, or negative one (−1) when the characterized nucleotides are AG or GG;
(b) assigning a genotype score for the SNP location rs17103168 of zero (0) when the characterized nucleotides are AA, or one (1) when the characterized nucleotides are AG or GG;
(c) assigning a genotype score for the SNP location rs5841 of zero (0) when the characterized nucleotides are CC, or one (1) when the characterized nucleotides are CT or TT;
(d) assigning a genotype score for the SNP location rs2396243 of zero (0) when the characterized nucleotides are GG, negative one (−1) when the characterized nucleotides are AG, or negative two (−2) when the characterized nucleotides are AA;
(e) assigning a genotype score for the SNP location rs1044457 of zero (0) when the characterized nucleotides are CC, or one (1) when the characterized nucleotides are CT or TT;
(f) assigning a genotype score for the SNP location rs1138729 of zero (0) when the characterized nucleotides are AA, or negative one (−1) when the characterized nucleotides are AG or GG;
(g) assigning a genotype score for the SNP location rs4643786 of zero (0) when the characterized nucleotides are TT, negative one (−1) when the characterized nucleotides are CT, or negative two (−2) when the characterized nucleotides are CC;
(h) assigning a genotype score for the SNP location rs11030918 of zero (0) when the characterized nucleotides are TT or CT, or one (1) when the characterized nucleotides are CC;
(i) assigning a genotype score for the SNP location rs12067645 of zero (0) when the characterized nucleotides are GG, one (1) when the characterized nucleotides are AG, or two (2) when the characterized nucleotides are AA; and
(j) assigning a genotype score for the SNP location rs17343066 of zero (0) when the characterized nucleotides are GG or AG, or one (1) when the characterized nucleotides are AA;
(ii) characterizing the subject having cancer based on a summation of the assigned genotype scores of (i); and
(iii) administering the subject cytarabine at a high dose, an agent that selectively binds to CD33, or clofarabine when the summation of the assigned genotype scores is less than or equal to zero (0), or cytarabine at a low dose when the summation of the assigned genotype scores is greater than zero (0).

2-3. (canceled)

4. The method of claim 1, wherein the summation of the assigned genotype scores is calculated by adding the genotype scores assigned according to the method of (a)-(j).

5. The method of claim 1, wherein the cancer is acute lymphoblastic leukemia (ALL), acute promyelocytic leukemia (APL), Chronic Myelogenous Leukemia (CML), or acute myeloid leukemia (AML).

6. The method of claim 5, wherein the AML is pediatric AML, or wherein the subject is less than 19 years of age.

7-8. (canceled)

9. The method of claim 1, wherein the assay is performed by DNA sequencing analysis, using a hybridization assay, using a Sequenom MassARRAY platform, or using a TaqMan genotyping assay.

10-15. (canceled)

16. The method of claim 1, further comprising:
(iv) performing an assay to detect the genotype of the subject for the SNP rs12459419, wherein the genotype may be CC, TC, or TT; and
(v) administering a therapeutically effective amount of an agent that selectively binds to CD33 when the subject exhibits a CC genotype for the CD33 single-nucleotide polymorphism rs12459419.

17. (canceled)

18. The method of claim 16, wherein the agent that selectively binds to CD33 is gemtuzumab ozogamicin (GO), hP67.7, SGN-33A.

19-20. (canceled)

21. The method of claim 16, wherein the agent that selectively binds to CD33 is conjugated to a toxin.

22. (canceled)

23. The method of claim 16, wherein the subject is treated with a chemotherapeutic agent within thirty days of the administration of the agent that selectively binds to CD33.

24. The method of claim 23, wherein the chemotherapeutic agent comprises daunorubicin hydrochloride and/or etoposide phosphate.

25. The method of claim 16, wherein the assay is performed by DNA sequencing analysis, using a hybridization assay, using a Sequenom MassARRAY platform, or using a TaqMan genotyping assay.

26. The method of claim 16, wherein the subject has one or more of: the presence of blast cells that express CD33 within the subject's hematopoietic system; leukostasis; anemia; leukopenia; neutropenia; thrombocytopenia; chloroma; granulocytic sarcoma; and myeloid sarcoma.

27. (canceled)

28. The method of claim 1, wherein the subject was administered a chemotherapeutic agent prior the assigning a genotype.

29. The method of claim 28, further comprising administering a chemotherapeutic agent to the subject concurrently with or after the administering of step (iii).

30. The method of claim 29, wherein the chemotherapeutic agent comprises daunorubicin hydrochloride or etoposide phosphate.

31-35. (canceled)

36. The method of claim 1, wherein the agent that selectively binds to CD33 selectively binds to amino acids encoded by exon 2 of CD33.

37. The method of claim 16, wherein the agent that selectively binds to CD33 selectively binds to amino acids encoded by exon 2 of CD33.
